SEQUENCE LISTING (27 sequences)

SEQ ID NO: 1 - DNA, 261 bp, Homo sapiens
aatgggctgatcaaagaaagaaggcatacgcagatttctacagaaactacgatgtcatgaaagattttgaggagatgaggaaggctggtatctttcagagtgtaaagtaatcttggaatataaagaatttcttcaggttgaattacctagaagtttgtcactgacttgtgttcctgaactatgacacatgaatatgtgggctaagaaatagttcctcttgataaataaacaattaaacaaatactttggaaaaaaaaaaaa

SEQ ID NO: 2 - DNA, 4344 bp, Homo sapiens
  misc_feature (1671) . . . (1682): n is a, c, g or t
taatattcgtgtgtataaatcgctcccgtgctgctctctgggggcccctctttcacaaacacctggccaccctcacgccacaatggccaggcaggaacctcgacctcccctcggaggactcagggtcaaccccggggtctcagtctctacatgtgacgttttcctgtcccctcatttaaaataacaagaggctgggcgcagtggctacgctgtaatcccagcactttgggaggccgaggcgggcgatcacgaggtcaggagatggagacatcctggccaacacggtgaaaccccgcctctactaaactacaaaaaattagccgggtgtggtgcgggcgcctgtagtcccagctactcgggaggctgaggcaggagaattgcttgaacccggaggcgaaggttgcagtgagctgagatctcgccactgcactccagcctggtgacagagcctgactccgtctcaaaaaataagaaaaaaaaataaaataaaaataatagaggccgaagcgggaggttcacttgagctcagaagttcgagatcagcctgggcaacacagtgagacctcgtttctatttaaaaaataaaataaaactaaatttaaaaaaatgcacgctcatagtacaaactttagaaatggaacgaaaaactaaaattgaaggtattcccctccaacccagagataacacctatcgtttattaagccctcactattgttaaacttagttttaaagggcacgatctcatttcttaaagacttctattccgcagaatttctttccaggcttttttctttttctttttttgagacggagtctcgctctgtcgcccaggccggggtgcagtggcgcgatctcggctcactgaaacctctgtccagtcttttcgaacccaaggcccaactgcgctctatctcgactttcggctccactcggatcccgaagtggcgcacgagataaaatgttgtcaggctgaggtaattctctgttagtcccggtaaaaattcgtcagtctggaaagctctcggtttggaattaaattctgtcactccggatggaaataagtccgcttaaggggggaaaatccgtttgtggaggacacgctcccgcacgtaaccccccgcggaaaatgaccccaagtacctttggccagggattgccgctgccacgccggactccatagccacggtcctgaaacgccccgccgggcaggccggaccaatggacgccgagctcggccgtgcgtcacgcgacgctggccaatcgcggagggccacgaccgtagaaaggccgggcgcggcgaggctgggcgctgggcggctgcggcgcgcggtgcgcggtgcgtagtctggagctatggtggtggtggcagccgcgccgaacccggccgacgggacccctaaagttctgcttctgtcggggcagcccgcctccgccgccggagccccggccggccaggccctgccgctcatggtgccagcccagagaggggccagcccggaggcagcgagcggggggctgccccaggcgcgcaagcgacagcgcctcacgcacctgagccccgaggagaaggcgctgaggaggtgggcgaggggccggggtgtggggccagatctgaagccgggactagggacaggggcaggggcaggggctgggagcggggacccagcactnnnnnnnnnnnnctccgtcgctttggctggcgggtcggtgccagcgtgggccggggcgggcaggaagccggactgaccggatccggggaaagaggaaagttccttatgatgcaagatggaatagtgtgtgtgtccagccccagtgctgtgacggggatgagtctgaggtggacggatgatgcaatataggagagaataaagcaggtcttcgagctagattgacagaagactgtattttttcttttgttttattgaggggaggagcctgaagtgtattttatcattagtctgtcttatactgtaaataaaaatgaaagcaccagctggtaaaagttttcaataaagaacataaataaggtttgataatgactcagtgtggtatgttccttctcttcctaggaaactgaaaaacagagtagcagctcagactgccagagatcgaaagaaggctcgaatgagtgagctggaacagcaagtggtagatttagaagaagaggtaaattccaagtcagaggaaggaaccaatatactgaatcaggtagatctaactgaataatagatgtttaatagcctctatgaacttctccagaaccaaaaacttttgctagaaaatcagcttttacgagagaaaactcatggccttgtagttgagacccaggagttaagacagcgcttggggatggatgccctggttgctgaagaggaggcggaagccaaggtaaatcatctcctttatttggtgcctcatgtgagtactggttccaagtgacatgacccaggcgattatgtttacagtctggactctgatccaagcagaactggcaaatatcttatctcgctctcaataataataatgttgttagataataaagttctatagcaattaaccctagaatcttttaaaagtaattctttaaagttgagaatcacagctgtctagcaagcatttccttgggccttaagcgtttattcactttggtctttcctccaggggaatgaagtgaggccagtggccgggtctgctgagtccgcagcactcagactacgtgcacctctgcagcaggtgcaggcccagttgtcacccctccagaacatctccccatggattctggcggtattcactcttcagattcagaggtagggatgttctgacttattaaagagctatataaccagttaattccatctgtttgatgcttgacatccctaactagacagatgagggttgaagttagttttccgtggggttggaggtgaacatcaactaccttcctagttccaggtaatatagaacatggagtgaagtgtagataaatgggtctggtgggtcccgaggtcgtacttatcacataatgactaatttacattatggaacccagtacaaagtgttccagttagattttccattgtattctgacagttgtacttcatttaatttttgcctcttacagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggacgtcatcagccaagctggaagccattaatgaactaattcgttttgaccacatatataccaagcccctagtcttagagataccctctgagacagagagccaagctaatgtggtagtgaaaatcgaggaagcacctctcagcccctcagagaatgatcaccctgaattcattgtctcagtgaaggaagaacctgtagaagatgacctcgttccggagctgggtatctcaaatctgctttcatccagccactgcccaaagccatcttcctgcctactggatgcttacagtgactgtggatacgggggttccctttcccattcagtgacatgtcctctctgcttggtgtaaaccattcttgggaggacacttttgccaatgaactctttccccagctgattagtgtctaaggaatgatccaatactgttgcccttttggttgactattacactgcctggaggatagcagagaagcctgtctgtacttcattcaaaaagccaaaatagagagtatacagtcctagagaattcctctatttgttcagatctcatagatgacccccaggtattgtcttttgacatccagcagtccaaggtattgagacatattactggaagtaagaaatattactataattgagaactacagcttttaagattgtacttttatcttaaaagggtggtagttttccctaaaatacttattatgtaagggtcattagacaaatgtcttgaagtagacatggaatttatgaatggttctttatcatttctcttccccctttttggcatcctggcttgcctccagttttaggtcctttagtttgcttctgtaagcaacgggaacacctgctgagggggctctttccctcatgtatacttcaagtaagatcaagaatcttttgtgaaattatagaaatttactatgtaaatgcttgatggaattttttcctgctagtgtagcttctgaaaggtgctttctccatttatttaaaactacccatgcaattaaaaggtacaatgcatccttgtttgatttcttctagggccgtaagtcttgtttctctccagatgtt

SEQ ID NO: 3 - DNA, 1166 bp, Homo sapiens
gcaagaatggtgcctgtcctgctgtctctgctgctgcttctgggtcctgctgtcccccaggagaaccaagatggtcgttactctctgacctatatctacactgggctgtccaagcatgttgaagacgtccccgcgtttcaggcccttggctcactcaatgacctccagttctttagatacaacagtaaagacaggaagtctcagcccatgggactctggagacaggtggaaggaatggaggattggaagcaggacagccaacttcagaaggccagggaggacatctttatggagaccctgaaagacattgtggagtattacaacgacagtaacgggtctcacgtattgcagggaaggtttggttgtgagatcgagaataacagaagcagcggagcattctggaaatattactatgatggaaaggactacattgaattcaacaaagaaatcccagcctgggtccccttcgacccagcagcccagataaccaagcagaagtgggaggcagaaccagtctacgtgcagcgggccaaggcttacctggaggaggagtgccctgcgactctgcggaaatacctgaaatacagcaaaaatatcctggaccggcaagatcctccctctgtggtggtcaccagccaccaggccccaggagaaaagaagaaactgaagtgcctggcctacgacttctacccagggaaaattgatgtgcactggactcgggccggcgaggtgcaggagcctgagttacggggagatgttcttcacaatggaaatggcacttaccagtcctgggtggtggtggcagtgcccccgcaggacacagccccctactcctgccacgtgcagcacagcagcctggcccagcccctcgtggtgccctgggaggccagctaggaagcaagggttggaggcaatgtgggatctcagacccagtagctgcccttcctgcctgatgtgggagctgaaccacagaaatcacagtcaatggatccacaaggcctgaggagcagtgtggggggacagacaggaggtggatttggagaccgaagactgggatgcctgtcttgagtagacttggacccaaaaaatcatctcaccttgagcccacccccaccccattgtctaatctgtagaagctaataaataatcatccctccttgcctagc

SEQ ID NO: 4 - DNA, 870 bp, Homo sapiens
ccgaatttcgggacaatcttcatcatgacctgctcccctctcctcctcacccttctcattcactgcacagggtcctgggcccagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgctggggtattcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataggttctaaaccctcaccccccccacgggagactagagctgcaggatcccaggggaggggtctctcctcccaccccaaggcatcaagcccttctccctgcactcaataaaccctcaataaatattctcattgtcaatcag

SEQ ID NO: 5 - DNA, 454 bp, Homo sapiens
tttttttgaaaaaaaactttggcatttttattcagacacgtataaaaacaaaacaaaaaacttcagtgatacaacagacgttttcccttagttccccatccaaggggacagaggtgtgcagctgaagctggatcttttttctgtcctacctggaagcttctcactgctggatgagaatggcttctaaaagtggatcttggggatccttgtgaatttgccctcggataaggagtgaagatcatttacggcacatgtggattatggtttacacaaagatgtccagttatttttccttctgactacccccaccaccacttcctgagatgagatgtctaggtataggaggatgtggctgttggggttagacttgattcagtgcaaaaacaagaaacagtgccccttaaacaagggctgtaacttaaatcagtttttcttggcaccttacaataagagc

SEQ ID NO: 6 - DNA, 79528 bp, Homo sapiens
ctccaggagctcagctccctgaaaggaaaatgatgccaagccaggcgcagtggctcacacctttaatcttagcaatttgggaggctgaggcaggtggatcacttgaggtcagaagttcaagaccagcctggccaatatggtgaaaccccatctctactaaaaatacaaaacttaggccgggcacagtggctcacacctgtaatcccagcactttgggaggccgacgtgggcggatcacctgaggtcgggagttcaagaccagcctgaccaacatggtgaaaccccatctctactaaaaatacaaaaattagccaggcgtggtggtgcatgcctgtaatcccagctacttgggaggctgaggcaggagaatcgcttgaacctgggaggcagaggttgcagtgagccgcgatcgagattgcgccattgcactccagcctggatgacagatccagactctatctcaaaaaaaaaaaaaaaaaaaaaaaaagaagaggaaagaaagaaaaaggaaatgacgccagagtgggcaggtccaacaggactttcagcccaaaccacacatggcacctgtgtccctcaccaatgcctggcctctggcactgagcttgggtcacctgtccacatctacttcccatggagtgagttatttcctttgaggcatgagtgtcctctctgccagctggacaggactgtcctgatgggtcagatagcagctaatcctatcaggagagaggaactggggcccaatctcatttctcttggcggtccccagactagccacaaatttgcccacctcctgccaggttaagagaaaggcagttttgagagttgattaagagcatggagtttcctgtaatcccagaactttgggaggccaagctgggaggaccacttgaggccaggagttcaagaccagcctgggcaatatagtgaggccccatctctacagaaattttaaaaattagctgaacgtggtgatgcgcacctgtggtcccagctacttgggaggctgaagcaggaggatcacttgagcccaggagttcaaggttgcagtgagccatgatttgtgtttcagccttagtgacagagtgacaccctgtgtcacaaacaaataaacaacaacaacaacaaaaggccaggcttggtggttcacacttgtaatctcagcactttgggagatagaggcgggtggatcacctgaggtgaggagttcggaaccagcctggccaatatggtgaaaccccgtctctactaaaaatacaaaaattagctgggcgtggtggtgggtgcctgtaatcccagctacttgggaggctgagacaggaggatcacttgaacgtgggaagtggaggttgcagtgggctgagactgcaccattgtactccagcctgggcaacaagagcgaaactccgtctccaaaaaaaaaaaaacaaaacaaaaaaaagagcatggaattggactgctttaggtaagttccttccccacctgtcaaatgaagtgataacaacagcaaaagaccgaatgttaatacctacggatagatcacttagcacagtgtgggcagatagtaactgctcaataaatgggaactactgttacatacctgtatgtagttctctaatatccctattttataaaaatgaggtccccccatatttttcttagcttccttttgtcaattaatatatcatagacatttttccaggtctgcacatgtagcctcctctatgtgagagccgcatggccttccaaaggacgccgtccatgcaagaggctaaactatgtctgtgggttcaaaaccctcagtctgtgccatggtgctttaaaacttggaaatggctcatacctgtaatcccaacactttgggaggccaaggtgggaggatcgcttgagcccaagagtttgagacaaacctgggcaacatgtgaaacactgtctctacagaaaataccaaaattagtatttttggtgtggtggcatgtgcctgtagtcccagctatttgggaggctgaggtaagaggatcgattgagccctggagttcaaagctgcaatagccataattgcactccagcctgggctacagagaagacctgtcttaagaaacacaaacaggctgggcgcggtggctcacgcctgtaatcccagcactttgggaggcccaggcgaggggatcacgaggtcagtagatcgagaccatcttgcactccagcctgggagacagcgagactccatctcagaaaaaaaaaaaaaaagaaaaagaaacacaaacaaaaacaaacttggaaattagttaaggtttagttcagttagatcagttaaagtttaactgttaaactgtcacctgcatttttatttctttgtctggaaatgtgtgtttgtctttctgttcatttgacttctaacccacatgggatgggttttcctaacttgggagaagcaggtgagaggtcagcctccacagggtgaggctgtcaaggaggaggggggaaccaggacagccagtggccagggcaggacaaaagaaggccggccatctttgttttgttttgttttgttttgttttgttttgttttgttttgttacgaggcagagtcttgctgtgcctcacccaggctggagtgcaataatgagatcacagctcattgcagcctccatctcccgggttcaagtgattctcctatctcagtttcccgagtagctggggctacaggcgtgcgtgtgccatcacacctggctaattttgtatttttaaaaattattttattttattttattttattttattttgaaacggagtctcgctctgtcacccagtctggagttcagtggcacgatctcggctcagtgcaagctccgcctcccgggttcatgccattctcctgcctcagcctcctgagtagctgggactataaccgcctgccacgacgcccagctaattgttttgtatttttagtagagacggggtttcaccttgttagccaggatggtctcgatctactgacctcgtgatccacccgtcttagcctcccaaagtgctgggattacaggcgtgagccaccgcgcccggcctattttgtatatttttagtagagatggggtttcaccatgtctgctgggctggtctcgaactcctgacctcaggtgatcgtctaccttggtctcccaaagtgctgggattacaggcgtgagccactgtgcccgcctttttttttttcttctttttgttgttgttggttttatttatttatttggacagagtctcactctgttgtctgggctggagtgcagtggtgcaatctcagctcactgtaacctctgcctcccaggttcaaacgattctcctgcctcagcttccagggtagctgggattacaggcgcccaccaccacacccagcttatttttgtgtttttggtagagacagggtttcaccatattgaccaagctggtctcaaactcctgatctcaagtgatctgcccacctcggcctcccaaagtgctgggattacaggcgtgagccaccgtgccgggccatgcgccatctttgtttctgttccagatgcctgggagtccttccatctcttccctcaccccccacagccaaccctttagcaaggacttttcctcaatatttctcatatctgcctcttccttccacccccacggccaccactttaattcagaacttggatcactgagatggcccctaatggtttcactgccttcttctgcccttccctagtccattgtccatatggcaacccggggcaggtgcagtttggaaaggaggtggcactacctggtaatgctgaagctgtgtatacttctagctccttcccctttgcaccagggcacttgcagtaggaaagtcacagcgacattgtctgcaaagcaagaaactggaaatgtccaggcacagcagctcacgcctataatcgcagcactctgggaggccaaggtgggtggatcacttgagcccaggagtttgagaccagcctgggcaacatagtgagacgcccatctctataaaaaaagaagaaagtggaaacaactcaaatctccatcaacaacagtggattcttttcctgttgctgctctaaccagttaccccaaactcagcggctgaaacacacacatttattatcttacagagctggaggtcaggagtccaaaatgattttactgaactaaaatcaacaagtcagcaggtatgcatttcttctgcaggctctaagggagaatctggtttttgcctttttttctggctttcagaagctgcctgctttcctgggttcatgggtccttccatcctaacgtcagcagtagtgggcaggcctttctcaggacactgtctctgattctgactcttctgcctccctctctcccatttaaggatccttggccgggcacggtggctcacgcctgtaatcccagcagtttgggaggctgaggcaggcagatcacctgaggtcaggagttcgagaccagcctgaccaacatggagaaaccccgtctcttctaaaaatataaaattagtcagacgtggtggcgcatgcctgtaatcccagctactcgggaggctgaggcaggggaatagcttgaacccaggagatggaggttgcagtgagctgaaattgtgccattgcactccagcctgggcaacaagagcaaaactccgtctcaaaaaaaaaaaaaaaattccttgtgattccactgggcccaccaggataatccaggatcatctcccatctccaggccgggtgattagcaacctttgttctcccttgccgttaaaggaacatttttgaaagtttcagagattaggatgtggtcatatttacaggccatttttctgcctccccacacagttacccacacagttacccatcgcataatagggctggggctgggggatgtgagcagaatccctccatacccaacatataaccacgcgaggcccccagctgagggcccggtgtgttgggcagagagctgaaggcaggggaacactgtccagtcgctgcacagcagtgtgaggaaaggaagaggagtcgtctgtaaaaacccattgactgtgctcatttcacactgcgtgcctctatcaaaacatctctagccaggcgcggtggctcatgcctgtaatcccagcattttgcaagggtgaggcaagcagaccacctgaggtcaggagttcgagaccagtctggccaacatggcaaaaccccgtctctactaaaaatcctaaaattagccagggcctggtggcgcgtgcctgtaatcccagctgctcaggaggctgaggcaggagaatcgcttgacccgggaggcagaggttgcagtgagccgagatcatgccactgccctctagcctgggcaacagagcaagactccatctcaaaaacaaaacaaaacaaaacaaaatctcatgtatcccatacatatatacatctattatatacccacaaaaattaaaattaaaaaaaagaaaaaaacaaaaaatccccattgactgtactctctgttatattcctggggccctcaaatgttgattacttttcagttttgagaatctaagtgacacctagcctggacaggaggaggggagagcccctgtttctggcaggtttctaagaggaatcacaggactgggctgagaggaggggacccagcacccaggctagagcagccacccctccatccctgagtgaggggccatctgctgccccccgcctctccctcgcccccacccccgcctctatctcaagccttgctcctttcccctcgggtgcctccacctgccgtttgccccgtcctcacagagtgtgaatcttgactcaaagtgtggacgtcaaagccccttggaagtgggaaaggcccaggcgggtgcagattgtttatttgcttgctgggtgctgggtgtgaaaggaacagacacgtgcttgcctcgtggggctcagtctctagtggggaagatgggcactgctgtcattgtaaagtgtgattttgtgaaaagtgctaagaaggaaaggacaggggctatgagagggggttgtggagtcctaattaggaaggaggagttgaactggtgagattgagggaaagcaaaaagcgaaagcaggtaagctctaagtgtgcctttcttcatgctccagaacacatgccctcctgcacaaataattcacaatcttcctgcaaccagccatcaccagacgctcagctgacagaaaattgcaaattagctcactgcaacctcggcactatcagtactgcgtaaagccctctccagcatacagcacgagcaccatcctgtcccgtccctagcaagcctctgtctccttgcagacagccccttctctgctgtgctgcctgttgcaaatttgcaacgcattttctttttctttttttctttctttttttgttttttgagacggagtctcgctctgtcgcccaggctggagtgcagtggcgcgatctcgcctcactgcaagctccgcctcccgggttcacgccattctcttgcctcagcctcccaagtagctgggactacaggtacgtgccaccactcccggctaatttttttgtatttttaatagagatggggtttcactgtgttagccaggatggtcttgatctcctgacctcgtgatccacctgcctcagcctcccaaagtgctgggattacaggcgtgagccaccgtacccggccttttctattttttttttttttttgagacaagttttcaccgtgtcacccaggctggagtgcaatggcgcaatcttgactcactgcaatctctgcctcccagactcaagggattctcctgcctcagcctcccaagtaaatgggactacaagcatgtgccaccacgcccggctaatttctgtatttttagtagagatggagtttgccatgttggccaggctggtgttgaactcctgacctcaggtgatctgtacgcctcagcctcccaaagtgctgggattacaggcgtgagccatcactcccggctgcaacatattttcataccttctctaataaatgtgcctttctttacctataactgtcttggtaaattcttcttactcctgggctactggcctcagatagttgccgctcacctgagacaggggtgacaagacggcttattcatagaaagcataatgaccaaattagaagaactgggatatccatcaccttaaacatttatcttttctttatgctgggagcatgcaaattattctcctgtagctatttttgaagtatacagtgggttatggttaactgcagtcaccctactgatctatggaacactatgtcttatttcttctagctaactgtatattgtacccattgatcagcttctcttcttcctcccctccttgctatccttcctggcctctggtaaccaccaatttactctccatctttgtgagatccacttttttttttttttttgagtcggggttttttagtagagacggggtttcaccatgtgagccaggatggtctccatctcctgaccgcgtgatccgcccatctcggcctcccaaagtgttgggattacaggcgtaagccaccacgcccagcctttttttttttttttttttgagacggaattttactcgttacccaggctggagtgcaatggcacgatctcagctcaccacaacctccgcctcccgggttcaagcaattctcctgcctcagcctcccaagtagctgggattacaggcatgcgccaccacgcccagctaattttgtatttttagtagagacggggtttctccatgttggtcagactggtctcgaactcccgacctcaggtgatcctcccgcctcggcctcccaaagtgttgggattagaggtgtgagccactgcgccaggccgagatcccccttttttttttttttttttttgagatgaagtctcgctctgttgcccaggctggagagtgcaatggcgcaatctgggctcaccgcaacctccacctcctgggttcaagtgattcttctgcctcagcctcctgagtaactggaattacaggcgcctggcaccacgcccggctaatttttgtatttttaggagagatggggtttcaccacggtgaccaggctggcaaagatccacttttttagtgttcatgtatgagtgaaaacatgcaatatttgtctttctctgcctggcttatttcagttaatatgatgaccttcagttccatctgtgttgttgcaaatgacaggatttccttcttattggtggctgaataatattccctttgtttatataccacattttcttcatccattcatccattgatgtgcgcttgggttgactccatattttggctattgtgaatggtgctgcaattaccatgggagtgcagatatctcttcaatatgctgatctccttccttttggatatataccgagtagtgggattcatctgaaccattttttttttagactagtcaagttcagtagtgagaagggagcaaaaagtagaacaaggggccaggcgtggtggctcatgcctgtaatcccagcactttgggaggctgaggtgggtggatcacttgaggtcaggagtttgagaccagcctggccaacatagtgaaaccccgttccactaaaaatacaaaaattagctgggcgtggtggtgggagcctctaatcccagctactcgggaggctgaggcatgagaattgcctgaacccaggaggcagaggttgcagtgaaccgagatcgcggcactgcactccagcctgggtgacagagtgagactccatctcaaaaaaaaaaaaaaaagttagccgggcgtggtggcgcacacctgtaaccccagctactcaggaggctgaggcaggagaaccgcttgaatccaggaggcggagattgcagcaaactgagatcacggcactgcactccagcctgggcaacagagtgagactccgtctcaaaaaacaacaacgaacaacaaaaaatacaattgcaaaccaccaccatttgacactgcctatatccacccccaaccctgctccagttgtttctttgcaattgccactttccagcatactatataatttacttttttttttttttttgagatggagttttgttcttgtcgcccaggctggagtgcaatggcgtggtctcagctcattgcaacctccgcctccctagttcaaacgattctcctgcctcattctcccaagtagctaggattacaggggccccccaccacgcccggctaatttttgtattttcagtaaagacagggtttcaccatgttggccaggctggtcgcaaactcctgacctcaggtgatccacccacttcggcctcccaaagtgccgggattacaggcgtgagccaccgcacctggcctaatttacttctttatgtagtctattgtgtatctatcttttccagggaacctcagctccacaagggcagggctctcttgtcactttggtttatggccatatcacagagcccatcccagtgcctggcacatattaaggaatgcattaggattccaaaaccagccaggagcggtggctcatgcctgtaatcccagcactttgggaggccgaggcgagtggatcacgaggataggagttcaagaccagtccggccaagatggtgaaaccccgtctctactaaaaatacaaaaaattagttgggcgtggttgtggacacctgtaatcctatctattcgggaggctgaggcagagaattgcttgaacccgggaggcagaggttgcagtgagccgagatcgcgccactgcactctggcctgggcgacagagcaagacgccatctcaaaaaaaaaaaaaaaaaaaaaaaaagattacaaagcccagaatgccacaagaccacaggtggtgcagtgaacagggcattgccttgagagtctatcatccctgggcaagtcattaaacataggggtgggggtgggagcctcctgtctgtaacgtggaggtgtcacgtgccaagggctcaggcacacgcgcgcgcacgcacaaacacacccattagtaaggacacgacccctggcattctaatgcgatttatacttttccatgaaggaattcatttccccctctcactaaccccagagacaggtagagccgacaggtggaagaactgaggcccgagagagacagcgggggaccctccccgcgccacggcacagcagctgtccgaccccctcctcacgcccggggggcgcgacggctgccgagggtcgcgggcagggggcgcggcgccggggactacatttcccaggagcagttgccgcggttccgccgttcctggggtttcaagagcctcgcgttaaagggggcgggggaggttgcgcagtccgccagctggaagatggctctgcgcccggtggggacccgagagccgcctgcctgggtgcccaggcggggtccggctgcagtgaagaagccccccagctctgcagtgtccctgaggggaggcgaggcgaggcgaggcgaggcgaggccaagccaattggaggccgtctttcctcgtcttgcccgcctcggcttcggcctctgcgcccaagccccagggaaccccctcccttcccctccaactccccggaacaggggaaggggcggtgaacctcgccgcctggcgggagaaggggtgtccactagctggaagcccctgcaacccgaggcaacccctttccccaattctggcttcagttccgggaatttacaggtcgctgggatgggcggggagaagggacaggagagatacaaaatgaaatgaacgcccaggtgtcagggagctgagccttacagtaccccgcccccaatccatccttctaagtcccgggcctccgcggtaaaataggggtgatggggatagtgatcaaaggcaggaggcgcaaaggatccagaaaccggtggggagcactgtgcaaactgtaaagcgctgtgcaaataaaattattgatcgtcgtgtcattggattcatccagtctttaagacatgcacggatctggggcgggggatggggtgacatggttctgggctctcagggtggaacatctcggatacggggagggggggctatttgggtgcgtattaacccaaccaggtcttaatgggaacccgagttgtctttgaaaataaccaaaacaaagccaccagaatgttgtgttttcaaatgacgaaacttaagagaaattgacaccatcaaaagacgaaccttgaccttttgaactaacctgaccccccattgcttcccttccccgcctgtgccacggagataagcccagagagaaggcatctgacaccgagtcttgcgctccccctcccccttcctcccctaatcatttggattaatccatcccttggcgtgcgggggaaacccattaatttgtcctggcttcagagactcccaggtccagtaggagccctcccacacccacgcagctacgatcccgtccccaacatcaaaaccctcgaacccctcccctttacccctccccagtagcccactgtgtctgcaaacagctcttggaccataccgggatgcgttcatcattttgaccagcaacaaactcccactcccagggtattgtttaagtgaacagtgaccggcctctatgcttagggaataaaagcggggttgtgggagaggggcccagggagcaaacgtcgcagaatctcagcccagggccgctgccaagcggacccggcacgtgagtaggaggtcggccgcagacaaggggtcctagggtctctgaggtctctcctgctggcctctgggcccctaaagcccctcgagcttcagaactccctacagacagtggcgccccgtgtccgcctcgtctacacctgccaagcagcacgggaaaatctgtgtgcattttaacaaagacatcgcatcttcccagcaaaatggcggtggtcgcggccacggctctctaaacaactccacactttccgaggacaggtcacaacccaagggacataaccccctcctcgacctctaccctagcgccccgctttcccaagtccccaaagcactcagggcctctcctcattacaggggccccccggaccgtccctggttagagcttctggggcagcttctccattgtggaccgccctcccccgcccccacattgccttcaaggagccttgggacggggactgaacccccaggctgggcctcacggatggggggcagcgagttctgtctctgacaccccaaaacttccaggctaagagaagcacacaaatgaattcccgattagggtatatgggagatgtaggggtctttgaggagaagaggccacccgggaggccctcctcatctctcagagggtccgggcagcactgggggttcggcctcaggagggaacctcaaggcagaggtcggggggcgcgctgggggagagacggcagaggagggggaagggacaaagtttgaaagtacccttccaactctttcagggaaagtttccttgggggccccatggacagagggggtagaggtgtggagggaccggtggcggggtgacccccgagaggtgaccggaccccgggggagcgacccctcccccctgtcccggctcggcccggctgggagtcgcccagctcggggccgcgtgtgttagttggggccgctttccggcagctccaccgggctaggggaccccgcagcgggccgggagcagagcccccggtagcctcaggggagagctttgctacggggggtttcccgcaccgaggctccctagccccgcagagccagccccccgcaaaggggaaatgtgccggcacaccagcggtcctcggcgccgtttggggtgcgtggcgggcgcggggggcggcggccgggccagcgggccgcccggcggggaagcccagggagccaggcagcggcccagggcggcggcggggagcaggaaggcccgggccggggggaaaggtcggatttgctcggcggaagaaacacagatggcggcggcgcagcgccattccgggccgggagcaggcagccagcagccctgtcctcaccgcggtccgcccgccgccgctaaatacccggatgcgccgcccaagcgccagacgcggagctgggaaaagggaggcagaggaggcggaggcagaggcagaggcagaggcagagcccgagcccggtgccgagaccaagcgacagaccggcggggctgggcctcgcaaagccggctcggcgagctctcccgacacccgagccggggaggaaaagcagcgactcctcgctcgcatccccgggagccgcactccagactggcccggtagtcaggggctcaggagcagatcccgaggcaggctttgctcagcctccgacgagggctggccctttggaaggcgccttcaacagccggaccagacaggccaccatgaccgagaattccacgtccgcccctgcggccaagcccaagcgggccaaggcctccaagaagtccacagaccaccccaagtattcagacatgatcgtggctgccatccaggccgagaagaaccgcgctggctcctcgcgccagtccattcagaagtatatcaagagccactacaaggtgggtgagaacgctgactcgcagatcaagttgtccatcaagcgcctggtcaccaccggtgtcctcaagcagaccaaaggggtgggggcctcggggtccttccggctagccaagagcgacgaacccaagaagtcagtggccttcaagaagaccaagaaggaaatcaagaaggtagccacgccaaagaaggcatccaagcccaagaaggctgcctccaaagccccaaccaagaaacccaaagccaccccggtcaagaaggccaagaagaagctggctgccacgcccaagaaagccaaaaaacccaagactgtcaaagccaagccggtcaaggcatccaagcccaaaaaggccaaaccagtgaaacccaaagcaaagtccagtgccaagagggccggcaagaagaagtgacaatgaagtcttttcttgcggacactccctcctgtctcctattttctgtaaataattttctccttttttctctcttgatgctcaccaccaccttttgcccccttctgttctgactttataagagacaggatttggattcttcagaaattacagaataattcatttttccttaaccagttgtgcaaggacagcaacaaccaatctaatgatgagaatgtacttatattttgttttgctattaacctacttacggggttagggatttgcggggggggcttgtgtgttttgttggcttgtttgccatgaaggtagatgtgggtggggagaagacacaaggcagtttgttctggctagatgagagggaacccaggaattgtgaggttagcaggaatatctttagggtgagtgagttttctttgagttgggcacccgttgtgagagtttcagaacctttggccagcaggagagaggtggtagggagcagccagccggcaaaggaaggagggggaaaaaaaccgccaccgggctgacttccacctcccagtggtgagcagtgggggcccaaacccagtttccttctcatttttgttagtttgcgctttcggcctccctattttcttagggaaggggagtggggtccaagtgacagctggatgggagaagccatagtttctcccagtcagctaggatgtagccattgggggatctttgtggcttcagcaaattctcttgttaaaccggagtgaaaacttcaggggaagggtggggagtcagccaagtgcctcagtgtgccctgttgaaacttaggtttttccacgcaatcgatggattgtgtcctaggaagacttttcttttcctctggatttttgttcctcctgtacaagaggtgtctttgcttggtttggtggggctgcggccacttaaaacctcccgatctctttttgagtcctttattataagtagttgtagctgcgggagggggagggggagtgggcgggcagtggatagtaagacttactgcagtcgatttgggatttgctaagtagttttacagagctagatctgtgtgcatgtgtgtgtttgtgtatatatacatatctagggctagtacttagtttcacacccgggagctgggagaaaaaacctgtacagttgtctttctcttatttttaataaaatagaaaaatcgcgcacttgcgcgtcccccccccacccccttttttaaacaagtgttacttgtgccgggaaaattttgctgtctttgtaattttaaaactttaaaataaattggaaaagggagaaactgagcggtgtatttttcctcactttgaagactggagaatgaatgcggagcggttaggcgggcaggcagtggggacatctggggcgtttgcacctgagaatggcgggggagggttcaggagctggacagaagccaggaatccttagtcccggaggctgcggaacgtgccttagccggcttctaggcttctgcctttggggtccagagggtaccccatttccggccgagaggtgagtacgtgggaatgcggtggggagtctggtgggcttggccctgacgcgacttcctccgcccccttccctcccctctgccatccggacttcggcagtctcaacttacaagtcaagcctctcggtccctggcggatcccgccaggccccgcccacccgtcctaggcccgcctccccgcctttttaagcgagcccccctagctccgattggctgctggagcccggcccccggccacgcgaccaggctgcgtccgcgatgcgcacggctcccaggcaggcaggcgcgctcgggcgaggtaggagcgatgtggcctgggaacgcctggcgcgccgcactcttctgggtgccccgcggccgccgcgcacagtcagcgctggcccagctgcgtggcattctggagggggagctggaaggcatccgcggagctggcacttggaagagtgagcgggtcatcacgtcccgtcaggggccgcacatccgcgtggacggcgtctccggaggtaacgctccgttccgggagtcgttccaagacctttcccgagcttgcgctggaatggaggtcctggaggtgggggtgggcggctcctacactcttagctactgttccccttcgcatctctcagggcgactttatggcttcctaatattgactcttgcctggttaaacccttttgacggagggaggcgatttgcccagcaagaaaacccgcatttcttgagcggctcctttgtgaaggtctctaccaattgttgctgcacgtgaactcctgctaacttctagacgcaggaatgactaattccactttacaaacgaagaggctgaaatccacagatgtgaaatggcttgcccaaaagcccccagctagctgcaggcagcgctggaatattggagctggaacattgcctgactggccagagaggacagcttgctgggtaaactcatagcaagcaaggggtcccagggctgccacagaaatcacatgatgagttggccctgccaggtctccctcatttctttctttccttttttttttctgagatggagtctcgtcttgctctgtcgcccaggctggagtgcagtggcgagatctcggttcactgcaacctctgcctcccgggttcaagcgattttcatgcctcagcctcccaagtagctgggactacaggcacgtgccagcacacccggctaatttttgtatttttagtggagactgggtttcaccatgttggccaggctggtctcgaactcctgacctcaagtgatcctcccgcctcagcctcccaaagtgttgggattacaggcgtgagccaccactcccagccctccctcatttcactctaaccagtggttacacctgacttattcgtgtcaggcctcaggtgagtggtaatcatcaacaagctcccaaagctgccttctgcccaggaaataggcatagaacccttggtgctgttgcactcatttgtaaaacagaggtcccaggtaatcccttctctccctcaggggaagcttagagccggtcaaaagatgggaaattaggagacccaagttctcatcctgtttttgcctttcccaggctgtgtgaactttcccctctggctgtttgctcctaaggtcagctgtgttcactctgggctttagaattcggggactggtatctcaggcacctacctttgggggtgtgattctggcagcaaagtaaactgtggagttccaggactgcagcaggcgtctgatccagccctcttctttaaccctcaaggaaaccaaggggtcagataaggagtcagtggtggtcttcaggcacaggagaagcctgtgaggaagggctgagttcctccttgggtagaatgtgattcaaagccagagtttaggctgggcacagtggcttgtgcctgaaatcgtagcactttgggaggccaaggcaggaggattgcttgaggctaggagttcgagaccagcacggacaacataatgagaccccatctctacaaaaaataaaaaaatttaatcgggcatggtggcacgcacctgtagtcccagctactcaggaggttaagtgggaggattgcttgagcctgggaggttggggctacagtgaaccatgattgcaccactgtactccaacctgggcgacagagctagaccctgtctcaaaaaaaaaaaagtttaaacttcttgctcttcaagatgtgtagcactgacgctccttctgtagctgttgaatttggtgcctcctcaatggtgactccaggtctgtttcctttctggaccatttccccagggcctggcactgtcatctttccaggcctgcccttgccccacctgagctgctgtatccatctcctctccttcacctcaggaatccttaacttctgtgccaacaactacctgggcctgagcagccaccctgaggtgatccaggcaggtctgcaggctctggaggagtttggagctggcctcagctctgtccgctttatctgtggaacccaggtacacagtgagtggtggggggttgtggggactgaccccagctgcctgcttcccacagtcagcaaggctggtttagccagctcagctcagccacagccctgggcctactgttggcctcagagctgtgcagcagaaaggctgagtgcaggcctctgttttgctgaaaagctttggccaaggaactttatatatatattttttctttttgagacagagtcttgctctgtcacccaggctggagtgcagtggcacaatcttggctccctgtaaccttcgcctaccagactcaagggattctccatgcctcagcctcctgagtagctgcgattacaggcatgctaccacacccagctaattttttttttttttttttttttgagatggagtctcgctctgtcgctgaggctggagtgcggtggcatgatcttggctactgcaagctgcgcctcccaggttcacgccattctcctgcctcaacctcccgagtagctgggactacaggcgcctaccaccactcccggctaattttttgtatttttagtagagacggggtttcactgtgttagccaggatggtctcgatctcctgacctcgtgatccacccaccttggcctcccaaagtgctgggattacaggcgtgagccaccgcactcggcctaatttttatatgtttagtagagatggggtttcaccatgttggccaggctggtcttgaactcctggcctcaagtgatctcctccaccttggccttccaaagtggaaactgcctttttgagcattcattttcccacctgcaaaacagagtccctcacacctgaccttctataagaaaagaggagtaggcggcaggaactgagactaagggctctggggctgttctgacgggagcacagttgccagtgctgccctttagtgtccctgttcctgtgggcaagtatcttaacttttctctgtctcaattacttcatagggaaaatgaagataatagcacttaataggatcattacaaattaaagacttaatacatgtaatgcttagaataggccgggcgcagtggctcatgcctgtaatcccagcactttgggaggctgatgcaggcggatcacctgaggtcaggagttcaagaccagcctggccaacatggcgaaaccctttctctacaaaaatataaaaattagctggcatgatggtgggtgcctgtaatcgcagccacttgggaggctgaggcaggagaatcgcttgaacctaggaggtggaggttgcagtgagctgagatcacgccactgcactccagcctgagcaacagagtgagactctgtctcaaaaaaaaaaaaaaaaaaaaaaagcatagaacagcctggcacatagtactgctcaatgaatgttggctattatttattttccaaatgttacaaaataaatttatataagtatgaaacttaaatagtacttattatgtgccaggaattcttttaagaaaattacaactattggccaggcgtggtggcttatgcctgtaatccaggcactttgggaggccgaggtgggtggattactgagttcaagaccagcctggccaacatggtgaaaccctgtctgtgctaaaaatacaaaaattagctggccgggctcgcttgaacccatgaggcaggggttgcagtgagccacgacggtgcgaccacactccagcctgggtgacagagggaaactctgtctcaaaaaaaaaaaaaaaagaacattacaagtattaactcattaatcctaagtgatagttgctattcttattcccgctattgagatgaggacactgagatccagagaggttaagtgacttgcccagaggcacacagcctgttgtgggatttgaaccctggctcaagagtccatgctggctgggcatggtagttcacacctgtaatcccaatacttttggagcccaaagcagaaggatcacttgatgcctggagtttgagaccagcctgggcaacatagcaagaccctctctctaaaaaaaaaaaaatgtttttaattagctgggtgtggtggtgcgcacctgtagtcccagctactagggaggggaggctgagttgggaggatcacttgagcccaggagttcaaggctacagtaagctatggtctcactactgcactccagcctgagtgacagagaatccctgtctcatttgccaaaaaaaagaaagagtctgtgctcctgactacttgtgattatcattaactgccctctcagaaaggtgagagtgaaatgaatgtcagaggcagaactcctgactcgcatccagtcacttcccagcaaagccctcacccagacccaacagttttcaaatttacaaattagaggcccaggtgaacccagaattacctctgtgatctttttcttcaccattaggcgaccaaaaaagacactcagtatggtgttaggaagacacatctttcttctccacgagtgcccgtaattccaccattagactgccactgtaaaaccaaagtctgcccagggagggaacctaacaggtccccagttcctcagtgggaactgtctgtcagattcctggcagctctgggatagcccatgtgtggcagaaagtaaggagactgaggctgcaggctttctgggttgggtctctgtggtcctccaagcctctgtcccctcgaacttgtcccacatgaccccacaggcatcaacacgtgtctcactcattttcttcagagcatccacaagaatctagaagcaaaaatagcccgcttccaccagcgggaggatgccatcctctatcccagctgttatgacgccaacgccggcctctttgaggtgtgtggaagctgtcctgcgggtgagggttggtggggcctgttaggctgaggaaggcttgggtgtgggcagtcgggagaacctagcaggattcagtgccatgcctagaggctccagagcccgcttctggaggtcaaggttgggccaggtaggtgctccaggaggaatcctggggcttgcagggagcctgagttccagagcctttgggggataagctacatcagctggaaggctgggggaggcgggggcctggtactcaccaaatatattctcacctcctcttactagagcagggcgctggtccagcccggcactgagacacgggggccccagagagaagcacagaacaccttgcctctctacccagaggagcgccctggctggcagtacagttttttgatttgctggagtcccggtcaagggagaagcccagggtggttaaatgatggctctacgctcactgcctccctccaggccctgctgaccccagaggacgcagtcctgtcggacgagctgaaccatgcctccatcatcgacggcatccggctgtgcaaggcccacaagtaccgctatcgccacctggacatggccgacctagaagccaagctgcaggaggcccaggtggggcgacgcctgggtccaccctcagcctccgcctggggaaggaagtgaggcttagagaggccaactcctcactcacagagatagcctgaaagtttgaaacagcagagatttgtccaagatgtgaacccagatccctcacacccagagcattattcttttttttttttccctgagacagagtttcactctgtcgccaaggctggagtatagtggtgtgatctcggctcactgcaacctccacctcctgggttcaagtgattctcctgcctcagcctcccaagtagctgggattacaggcatccgccaccccacctggctaatttttgtatttttagtaaagacggggtttcatcatgttgtccaggctggtctcgaactcctgacctcagttgatttcacccacctaggcctcccaaagtgctgggattatagacgtgagccactgtgcctggcaccagagcattattccattgtgccatggtgccttgaggtttttccctgtgtatgtgtgacagtctcactctgttgcccaggctgtattgcaatggtgcagtcatagctcactgcagcctcaatcttctggactcgagcgatcctcccacctcagtctcctgagtagctgggactaccggtgcccaccacacccagttttttatttatttgttttttagatggaatttcactctttttgcccaggctggagtgcaatggcgtgatctcagctcactgaaacctctgcctcccaggttcaattgattctcctgcctcagcctctggagtagctgggattacaggcacacaccaccgtgctcagctaattttctatttttagtagagatggggtttagccatgttggccaggctggtttcgaactcctgacctcaggtgatctacctgtctcagcctcccaaagtgttgggattacaggcatgagccaccgcacccagcctgtttgtttgtttttgagacagggtctcactataatgctcaggctagtcttgaactcctgggctcccaaagttacaggcatgagccaccatacccagccagtttttaaaatttttagtagagacagggtctcactatgttgcccaggtgggtctcgaacccgtaagctcccaaagtcctgggattacaggtgtgagccactgtgcccggcctgccttgagtattagcaagcaccttctgtgtggcaggcccttgctaggcataaggatagaatgtacatgttggtgagacctggtcccagcctagcctgccctctgggaccttacacaggagactgagatgtgagcccattctcactgtccagagctcagggtggagatgggtgacgctgagaggcacagatgagggagaggatgttccagcccccaagatggtggtacctctgtgcccacctctgtgctgggcactgggcaggatgagctctcagagctccaagcagcacaagagacgggtggtttggcccaacttgacaccacccaccatctgctcatgtctttcctgcagaagcatcggctgcgcctggtggccactgatggggccttttccatggatggcgacatcgcacccctgcaggagatctgctgcctcgcctctagatatggtgccctggtcttcatggatgaatgccatgccactggcttcctggggcccacaggacggtgggaccatgtggcacctgaggcctggggtggggcttctgagggtggctgggaaacgaggaggccagtccccagcatgatccataatccctgggctctcaggaggccagtgacagcagcggtggcttcccttgcaggggcacagatgagctgctgggtgtgatggaccaggtcaccatcatcaactccaccctggggaaggccctgggtggagcatcaggtacctgcaaggttgtgtccctggggtccccttgtccttttgaagggccctggaggggctcagggaagtggggagggcagcatggactgtactttcaggagggggttgggtaggctctggcccctgaccaacccctccccccacctcttcccttcttctcagggggctacacgacagggcctgggcccctggtgtccctgctgcggcagcgcgcccggccatacctcttctccaacagtctgccacctgctgtcgttggctgcgcctccaaggccctagatctgctgatggggagtaacaccattgtccagtctatggctgccaagacccagaggtgcgactcccagcagggcaggctcgggggcggagagacgtggtgagagccagcctcatgtgtctgcccaggcccacagacagctctgtgcccaccgggtctgcttctgcctgttcccctcccttctctctgtccctgcctctcccctctagcttctgtgtctccttcttatccctcctgtctttccacctcccttgttgttctccctgccccctgcctggctcccctctgcctcttagagcttgtaactgtctttgttgatccttcttgcagacttgggcatagacctcgggcctggtccctgcaaggagcgggtgtgaatgctccacggccccttagctacctgtgacaccttgtgcccacaggttccgtagtaagatggaagctgctggcttcactatctcgggagccagtcaccccatctgccctgtgatgctgggtgatgcccggctggcctctcgcatggcggatgacatgctgaagagaggtaagggtgctgagacaagggaactggtggtgggtcctgagagaagagaaagggaaacccctagactgtgacccagccccgtacccagccacgctgcggcctcttaggccagctgtctgtctaagcttgaacaccccaagccatgaggcagtcccgaatctgagacctggggcagttcccttgccctctgtgttctgcccccaagcctgttggttctggcacgcccttgctttctaccctcccaggcatctttgtcatcgggttcagctaccccgtggtccccaagggcaaggcccggatccgggtacagatctcagcagtgcatagcgaggaagacattgaccgctgcgtggaggccttcgtggaagtggggcgactgcacggggcactgccctgagctctgggtaaggacgagaagagccaaggtccgcctactgccacagggtcaaaggaggttttcgatcagcccagaccagaggctctgagccctgaaccaaagtcccagagctgggctgggacgtgacctgtgctgagggctgtgagaatgtgaaacaacagtgtgaaaattggctgtgacactctggtctgctttattgtctctgggcaggcccagtcctgtggccggttgaagaatcaggcaggagccagggctctgaggggaggcgcctgaggactgcagatctccactgacctctttccctagattaagatgggacccagtggccgggcacggtggctcaggcctgtaatcccagcactttgggaggccaaggtaggcggatcacctgaggtcgggagtttgagaccagcctgaccaacatggagaaaccccgtctctactaaaaatacgaaattagccaggcgtggtggcgtatgcctgtaatcccagctgctcaggaggctgaggcaggagaatcgcttgaacccgggaggtggaggttgcggtgagccgagatcatgccattgcactccagcctgagcaacaagagcagaagtctgtctcaaaaaaaaaaaaaaaaaaaagctggcacccaggcctttgggagataggcccagtccaccgtttgtctcgggtagtttctgtgctgctgtggagggctgctgagaactagccccacaggccttttaccctgccactgcccgctactctgcagcccctgagtctagtcttagagtctagcttcttccttctccccactggaagatgtcctgtagctgcccagataggaggaggctgggtgtccatgtttggaaggagaggcagaggattcaggcccaaaagaaaagaatgaggatcacccctggggaagctccagacagtctggaaccctgtgttcgcgcccactctctccctgccttactctggctttcggcaagctccagtccctgtgtgtgcctcagtcctcctgccactatgccttgaggtctgaggtccttccatctcgctcattccaaggcctacgatgatcagccaccagggggtgctgcgagcacgggccgcggccgccggctcgccccgcccctccagactgggggccttgggccgcggctgttcaggctgcccagcagtcaaggccaggagaggcgatttgctgctgccaaggccccatcctcccttgtgccccacaacggctcgccgctttcctgttggaggggcctgcgggaggagggcggtctctccctggcgaccttgtggaccccttgtggggtggggagtggggtccccgcagctccaccctcagcgtggtgtgaccttgggaagctcttagtttgcttgggacagaggacgaaaaggccctttatcattaacgacatgaaactagccctacgtctctctctgacgtcctgcacctccagcccacccatttgcctgttatcttgggatgaacaatgtaatatgagctgggctctggcccagccctgcaggatctgaaagctgggattcagggaatcccagcctgcgatccggaggaccttgctgctggggtccaggagctgggggagggggggtgtggggggtcggtgtgagggtggaaggcagcctggctggggtagctgtggccacattagcggcaggaacatttgcagcccaccaccaccggctagggcacgtggcaccaggcgcgctgctgcaggggatgtgcacaagggctcaaccagtaatgtcgagccagcagctggatgagcatggtctgctctagccagcagctcatgttaagcgccacgggcagctgcggggtgacgtgtggcagtcggtgtgctgcacccggcggggccagaacagagggcccaggtccacccagaccgacgtgaggcggcacgaggcgagatccagacagcggcgcagaaagtgcgtcgccgcccctgtggctcccaggagccgccgctggcgttgccctgctgcgctggcagcgccgcgaccccctgggctagccgatcgcggtagggctccacgccgttggccaacccgcggctcagcgtgtcctgctgcaccagcccgggtccgccgccgcgcttcttccgcagtcatccagaaggtgtctgtaggaggcgcagcagccgctctgggtgcacgtgtttcggcttgggcgcctaccggtagtcgccggccgcccacgacaagctgtagggccacgcggggcgggagggccggccgtggaccagctgcaagtctgcgttctgcagaatcagaggctctgaggaccgccccaagggcgcaatgtccttgttctctgcgtcgcgcacggggttcagggccgcgggctaccagaagcggcagtaggagtggatggaggtggcaccgggcgcacccaaggtctaggctcacggccgcgctctttatggccgcctgttccttcctccttccctccacctccgctctcatatcagcttagccttgccgctgctgggagctcatcgccctaggccaagaaggaccttgtgggcagggggctcccaagggccaggagtgcgaagtcatgaatctcagatgctttccagcccgttccagggttcaggcatgcacattcgtccaggcttctgggagtctaagggtgcgttactttcacgcagtggttgcaggtctgtcaaagtttctaaaatcccaccaagctgagtctcgccattcagggatcataagagtctacaattcctttgcgcaattcaattctgaattcaattcagaattaattcaattctggccgggcgcggtggctcatgcctgtaatcccagcactttgggaggccgaggcgggcggatcacgaggtcaggagatcgagaccatcctggctaaaacggtgaaaccccgtctctactaaaaatacaaaaacaaaacattagccgggtgtggtgggcgcctgtagtcccaactactcgggaggctgaggtgggagaatggcgtgaacctgggaggcggagcttgcacttgcagtgagctgagatcgcaccactgtactccagcctgggcaacagagcgagactgtctcaaaaatatatatatatattaattcaattctgaagcttctgaattcaggtttggcaagtctagattagattcctaatagtttcaattccgtgagactctgcttccaaaaattgaggaatttctgcatctaaagaaggtgacatcaggctctatagaggcacctctgagccctcgtgaaaaccccggggcctttgaggagctggttcttttcttttctttttttttttttttgagtcttgctctgttgcccaggctggagtgcagtggcacagtctccgctcactgcaacctccgcctcccgggctcaagccattctcctgcctcggtctcccaagtagctgggattacaggtgtgcaccaccatacctggctaatttttagtagagatgggtttcaccatgttggccaggctggtcttgaactcctgacctcaagtgatccaactgccttggcctcccaaagtgctaggattacagacatgagccaccacgcctggccttaaggagctagttctgagcatctaccccagctcctccggcctgggatgcgagtcctggctccatcagcgcccctcacactgttttctctccccttacgcacttccccttgatgctccccaaccccagccccagcttctaaaaacactacactcagcccctccacctctcccctcccttccttaactcactgcagactgccttttgccctcaaaacacctgtccagtttaccagcagcctcctcattgctgaatccagtgtaccttgctccccaggtgctagcctccctaaccctctggaggcatttggccatgtggcccctcctccacacagttcaggacagaactccatgaccaaatcacacagccgttcaccaagacctattctcctctttggctcttgcctagacgagaggctcctaacaggtcactcatcaaatctctcccactctgtccatccttttcaccccgggcaataatagacttttttttttttttttttttgagacagagtcgctctgttgcccagtctggagtgcgagcgatctcggctcactgcaaccaccacctcccaggttcaagcgattctcgtgcctcagcctcctgagtagctgggattataggtgtgagccaccatgcccagctaatttttgtatttttagtagaggcagggttttgccatgttggccagtctggtctcgaactcttgacctcagatgatccgcccacctcagcctcccaaagtgctaggattacaggtgtgagccgctgcgcctggcccataacaaactttttacaaaagataatgtggggggaaaaaaagaaagaaagaaagaaaatgtgcgggtcatgtctctgctgtagaatcttagtagttccccgttgctatcaggtaaagccagactttttggacaggtacctaaaccatctgggctccagctggactaaggctctggaaagagcttccggagaaggatctcaggatttgcagagcctgagggaggagtctctggagtctgccgaaaggagactcaagtcctggtttcccactccaggaaaccccagtcaagactaggaatcctttttttttttttttttttttttttttttgagacggaatctcgctctgtcgcccaggctggagtgcagtggcacgatcttggctcactgcaacctccaacccctggcttcaagcaattctctgcctcagcctcccgagcagctgggattacaggtgcccgccaccacacccagctaatttttgtatttttagtagacatggggtttcatcatcttggccaggctggtcttgaactcctgacctcatgatccacctgtcttggcctcccaaagtgctgggattacaggcatgagccaccgcgcccagcctcttctttttttttttttttttttttttgagacaatgtctcactccatcacctgggctggagtgcagtggcgtcatctcggctcattgcaacctccacctcccaggttcaagcgattctcctgcctcagcctccagagtagctgggattacaggcgcccgccacgacgctcagttaatttttgtatttttagtagagacaaggtttcgccacattggccaggctggtctcgaattcctggcctcaagtgatctgcctgcctcagcctcccaaagtgctgggattacagacgtgagccaccatgcccggcctaggaaccttttctaaacatggattggagcaagaaggtgcagggataggggatggagtcacatcaggctgggccacaggaggtgggagtgccagagactggctgggccctgggcacagaggggttaactacaaagcaaatcacagagagcgagatcaatgggttactggctgcagagagaggaaaaggcagcagcaacgcagagaggcagagaagagccaggcagaagagcctgccacctgggggaaatcagggactgaggaggaagtggccagtggctactccagagggagtgcccaactccggctcagcagagggggcagaggagaggaggggccttaaaggcaaagggggagaagtagaaggcagaccagtacctgcagggaggcaccaaggggcagccgctgaatcaggaggcccacccagcccggccattccacccaggatcaagcccactgcccaaggcggaggtgggtgttgcccatcctgggctttggggacctactgcatgctgggtgctgggatgggggcagacattgcctttcttccctactccctgcctgacccccctttcccctactttgtcccaccaccccagtggtctcaagcctgcaaggagatccacaggcagtaggagacatgggctgtagagtgggatgggggtgagcatgggtgcggtgtgcacacacacaagttgtcagaggcaccacctgttacaagctctcagatgagcactctagcttaagaggcaggataccagggtggcaacagccccagagctgccatttcgagtgtgatattatcctcccatgtcccctctctgggcctcaatttccctatgtgcagagaggaagcagtgacctacacggtcttcccagttttgccattgtgatgatctcactatactggaccctccaatgactcagggatgttacccgtgttttgctaaggctacagcttgtagccagagaaagccctaaacccatgtcccagggtctcccctgagcttcagatccccattgagaaggaaaacataccagtttctgggtttggcccctccccccatcgccatatcttcatcctacttcgcttgtgccttccccagcctcgtgggttgaaagtgtccctcaaatacctaggtagtcctccaggcaggactccaggggtgttccttaggaaactgaggaactctcaccccttgaacaccacgagcttgctcacacagtgacactcagcgatgactttggctctgctctcccctcctccatctcccacgagcttccagcccagaacacctggccagacccaggtcgggggagttagatcccggggtcaagcaaccagaactgggggctcttgcctgaggattccagcttctcttcccaggtgcccgtctgatggggagatggctgatgcccagaacatttcactggacagcccagggagtgtgggggccgtggcagtgcctgtggtctttgccctaatcttcctgctgggcacagtgggcaatgggctggtgctggcagtgctcctgcagcctggcccgagtgcctggcaggagcctggcagcaccacggacctgttcatcctcaacctggcggtggctgacctctgcttcatcctgtgctgcgtgcccttccaggccaccatctacacgctggatgcctggctctttggggccctcgtctgcaaggccgtgcacctgctcatctacctcaccatgtacgccagcagctttacgctggctgctgtctccgtggacaggtgcgctgtgcctggggcctggctgggcagggctgtgggggcgggggttgggggaggagtcctgaacagatcctcactggccttaggaaggagagagtgggggaccagaaagggaggtgggtgggaggaaacaaaagctccctgacccctcgcaagcagcctctgggcacctgcagggcgtgcttgaggggactgtcctgcccttcccctcctccactgtgaacttccagaggacgcctctgagtctcaagtggcagcacagggtctggcacatagtaagtgctctgtaagcgcgaaatgaatcgcaaaagaagctcacgaatgcgttcatcagtttttttgttttgttttgttttgttgttttttttttttggatcttggctcactgcaacctctgcctcctgggttccagcgattctcctgccacagcctcctgagtagctgggattacaggccaccacacctggctaattttttgtatttttagtagaaacggggttttgccatgttggacaggctggtctcgaacccctgacctcaagtgatccgcccgcctcggcctcccaagtgctgggattacaggcgtgagccaccgcgcccagcccagctattttctaactgcccacacctggccaagctgtgcacacatctgcttccacagcttgaaacttggggtcaaatccaggctcactccagctgatgaccctgggcaagtcacttctctctggacctcatctgacgcatccataaaataatcctagaaataacaagtcaccgggatcgggcccttgctaggtgcaagggcctaagcaccttgcgcgttcacacccttaatccccgccacgtcccccacggttcacaggaggcgcactgggccgcagggcccgggcgcgggacgtggcgcgggcccctgcgggagggcacctgcccgccccgctgaccaggcgccctccgcaggtacctggccgtgcggcacccgctgcgctcgcgcgccctgcgcacgccgcgtaacgcccgcgccgcagtggggctggtgtggctgctggcggcgctcttctcggcgccctacctcagctactacggcaccgtgcgctacggcgcgctggagctctgcgtgcccgcctgggaggacgcgcgccgccgcgccctggacgtggccaccttcgctgccggctacctgctgcccgtggctgtggtgagcctggcctacgggcgcacgctgcgcttcctgtgggccgccgtgggtcccgcgggcgcggcggcggccgaggcgcggcggagggcgacgggccgcgcggggcgcgccatgctggcggtggccgcgctctacgcgctctgctggggtccgcaccacgcgctcatcctgtgcttctggtacggccgcttcgccttcagcccggccacctacgcctgccgcctggcctcacactgcctggcctacgccaactcctgcctcaacccgctcgtctacgcgctcgcctcgcgccacttccgcgcgcgcttccgccgcctgtggccgtgcggccgccgacgccgccaccgtgcccgccgcgccttgcgtcgcgtccgccccgcgtcctcgggcccacccggctgccccggagacgcccggcctagcgggaggctgctggctggtggcggccagggcccggagcccagggagggacccgtccacggcggagaggctgcccgaggaccggaataaaccctgccgcctggactccgcctgtgtccgtctgtctcactcccgttctccgaaggcgggacgccaccgggccagggatggggcaatgccacgagctctctgaggggcgttgagtggagcgacttgtccccgctccacagcggagcaccagcccagtggacacccctgggtgtgaatatgaacagcacagcccgcccccagcagggcagcccccagagcctgcccagagccagcctggcagtccctctgcccattcttccaactgggtgacacctaggccctgcttcctccaccacctcttaactatctccttcttggttccaagctcagtcctaggtaatttaacatcaacagctaataaatggctaatatttaagtgggaccaggcgcttggctcatgcctgtaatctcagcgttttgggaggtcgaggcgggaggatcgcttgaggcagcagttcaaggctgcagtgagccatgatcacgctactgcattccagcctagacagcaaagcaagaccttgtctctaaaaacatttattaagcaggtaccatatgcctcatgctacattctttgtcaaaacagctttgcaaatagatatgattaacctttcctttttttttttttttttttttgtcttttagagacagagttttgtctcccaggttggactacagtggcgccatctcggctcactgcaacctccgtcccccgggttcaagcaattctcctgcctcagcctccagagtagctgggattacaggcgcccaccaccacatccgactaatttttttttttaatagagatggggtttcaccattttggacaggctggtcttgaactcctgaccgcagatgatccgtccgcctcggcctcccaaagtgctgggataacaggcgtgaaccactgtgcccggctgtttagccccattttacagatgtagaaatcaaggatcatagagatttagtgatttgccacaccgctggtgtttggcagaggttggatacaaacccatggctgtgtaactttttctttttttttctattattattattattattatttttgagagagagagagagattgttatccaggctgcagtgcagtggcacaatcttggctcaagacaacctccgcctcccaggttcaagcgattctcctgcctcagcctcccgactagctgcaactataggcgtatgccaccacacccggctaatttttgtatttttttattatttttattttttggtagagatggggtttcaccgtgttggccaggctggtcttgaactcctgacctcaagcgatccgccggcattggcctcccaaagtgctgggattacaggcgtgagccaccgcgcccggcccagaggtaacatttgacatgtattttcttggacaaaggtggggaggggcatgccaggtagagcaacagcctgtgcaaggcaggggacgtgcagggcagtaagtggctcctgcagctgaggcagggagagattggggcaggggaggcctggagggcaggagcttcatccttcaggcagatggtgatgccactgaaggttttcaggagaggagggggcctatcagatgtgcagttcagaaagctcccagggctgctgtgtacaagctggaatggaggcctagacttggcagaggactccgatcaggtgggttctcctgggcccttcttggaagtgaggagactggtaaagagtctgctccgtgagctctgttgaggaaagccaacgaggcctggcctaggactgggaagatatttttgccgtggacttggcaggaagatggatggcctgctgccaggcttgggcttggttcccgaggctccgaggtttgcttcagggggctaatgaaacacttggaagagcagatggtcgtaataataataactcaggagagaattgtgtgtccagtagagcccaaggcaggcaagagggtcggagcggaggctgaattgagtcctggccactgctccccagggcctgagagaaccctcctgatctgagccctctgtcaagtgtagggctgagcctggggcagccctggggacctggaaaggcacttttcttagttggaaggaggtttggattctacaaccttcttaccttcctgtttgtgcaaacctcttaagaagaggcctggccggatcacgaggtcaggagatcgagaccatcctggctaacacagtgaaacaccatatctactaaaaatacaaaaaattagctgggcgtggtggcaggtgcctgtagtcccagctactcgggaggctgagacaggaaaatggcgtgaacccgggagacagagcttccagtgagccgagatcacgccactgcactccagcctgggggacagagcgagactccgtctcaaaaaaaaaaaaaaattactctttagggggcctggcgcagtagctcacgcctgtaatcctagcactttgggaggctgagacgggtggatactaggtcaggagttcaagaccagcttggccaagatgctgaaaccccatctctactaaaaatacaaaaaaaaaattggtcggccttggtggtacacacctgtaatcccagctactcgggaggctgaggcaggagaatcgcttgaacccaggcagcagaggttgcagtgagccaagatcgcgccactgcactccagcctgggtgacagagcaagactctgtctcaaaaaagaaaaaaaagatttactctttagggagctctttctggctgcccaggcaggctttcatagggaggaagtacagagtgtgctgactggagcagagaggtcagtcaggaggctcgtgtgagaatccagagagaaagacatagaggtattggaagtggtggggagacatggacagatgggtcagccacttaggaggtacaattgctaggacttggtgactgttggagggtaaggaggagagacgtgtcccagatgacaccccagacttctggcttgggctatttgggggtggcagtggggataactgagggggacagttttggtggtcatggtgggggtgcccgcagaatagcggtgtactctgagtacgtgccacgtttcacaaccactgtttgcaggtgggcaagggcagggagcttgtcctttcattccctgctgtcctcccacctagaacactgcctagcacatagcaagcactaagtcaataagtcaatatttatttttatttttatttttattttttgagatggagtctcactcttttgcccgggctggggtgaagtggctgaatcttggctcactgcaacctccacctcctgggttcaagtgattctccggcctcagcctcccaagtagctgggattacaggtgggcgccaccacgtctggctaatttttgtatttttggtagagatggggtttcgtcgtgttggccaggctggtcttgaactcctgacctcaggtgatccacctgccttggcctcccaaagtgttaggattacacgtgtgagccactgcgcctagcccaatatttctaacatgaaagaaacacgaagtataaggcaaaggatcttaacccgcaaggtgaaatcaccaaatgtttcttcttcctgccatcttctaagccttcctccgcacctccccgactcttcagtaattcgcagagcagccagcaaccagcaaaactcacccctgtgagtacaacctcagctgagctctctttcctgccctggcccatggccagctcagggaccactgagctctctatcctgcccagtccagccagctcagggaccactgagctctctatcctgcccagtccagccagctcagggaccacttgggtgttttgtgactcactccagtgtctgaggccgacttcatctggctggctcttcagacacaattggggttacaggaaatatcctggctggaaatggggtgaggcagattggctgtctacatggctgtctggcaagtgtttgatgactgacctataagccaccttcagtcaagcctttggttcactgtctacatggccagggagggagtcaacaatagacactccacctccgcaacggccagttctcctggatccatggctccagggatggaggatgctgcaggagacacaggccttgcttcccagctaaggactgtgggtcatctcagggcgatttcacagtcccagcatgccccagctctcagctctgcaaataccaagcagcctgcctaggggacagtgggctcaggagtgcccttggcaggtcctcacctatctgctcccacagctctgtagcaagggttctaaccttttttgagtgtgaagcctgctgagaatgagaatgagagctgtggacctgtttcccagaaacgcatgtgtgtactctccacacaaaaccttgcaccgtttcgggggctcacacctccctaagggctcgctaattgaaccccttgggcctgaggttgagaaaccttgcctcaattcttccccaggtgatcaggccagggtgagagaggacagacctggcaagcaggacccatgagaaagcccccttcctggactttgaggcccgctccctcctgcccctgcctcccctctgccctggactcctctctgctctgccccattcccagggccatggccatgggggggctgtgtttggatacaatggcaaccctgggcccagaaagtgggcaggctcacctggaggggatcagagtaacatggcaggaagcgagtgaaaagctaccccagaaccgcacctctgctctccatgtgactggggtgccatcctcccttacctacctcaggcagtggcctgagttccacgggcgcgccccacagctccctctgccgcctctcttcaggcttggggccactgcactttcccttccccagccctgccaacctggtggggcactgggcttccctctcacagggtcctggggacaggcctaccctacatctcatacccacagacagacccttttttcctccaaggcctggcctaggttctatgagttcaatgcagatctgagccataccaagctgggtttggggaacacactctcctcctctgaaaggtagacagggattccaacttggtgaaggggagagtagggcagagccaggcgggacacggactgatcacctggagaaagtcaccatcagcagccttggggcaggggcgggcccagcagtgccatcccctgggttttccctgagcaggcctccgctaggagcgcacgcttccttctgcctgtccatggcccacagcaaaggactggtgctcctgcagcggggagcggggctgctgacaggactgaacctcaaggtgagtttttggggcttcactacaatactagtttctggttgctgctgcaataaattaccacacacttagtgacttaaaacaacacaaatttattcccttagagttctaaaatcagccagaattataaaattagcctcactgagccagtccaggtgggagcagggctggggccttccagggctctgtgggaggatgcatttcctggctctgaggtggcctggactctgtggcttgagctgccctcctcaactccagtttcctgctccatcgctacacctccaacctcctctcccgtcacctgctcttctgttatacagacccgggtgaggacatcaagccctcctgaacaagctgggataatcttcctggcgaaagatccttaacttcattacatctgcaaagcccctttggccatacaaggtaattttgacaggttcctgggattaggacatgggcatcttgggaggccactactggcctaccacaactgggcagcaaaactattacaccctccggtataatagttttggtgtttcaatgactgggaggaaaagggttggaattttttgctttggggtccctcttaaccttgtatttttaaggtctgggactcaccaaccctccccttccaaccagagaaactcactgcagtatctccttgaaagtctggtgacgtgtctgtctaagtgctggtgagaggcacaggaccaactgagggcctcacgcagtacccctggccacagggtgcccgcctaagcccacacctgctgactgcaacaagcccatggccaggacggaacacagagaagggtctgaggcctggagcgcgccatgaaggccatggggcaagtgaggtctgctgagatagcgcaggtctgcggagctgaagtgcagctgcagaggctggtctggtgctgcgagaactggaagaagctgggagctgtggtccctgtccacagagatgctggaaactgtggctgggagtggctctgaggccgtggagagagagcatctctgccccacggcatctgcgggtgagggcaaggtcctcaccagacaagtgcagctccaagtcccagatgttgggctttttcttggtactgagacagcagtggggcagggtggggcagtggcaggaaggcagggagcctctcttgctacctcttctccatgctctgcatctgcagactgagggaggtgaagctggccaggaggtcagtcacttcatccacctgcaggacggaacagaggctctgttatgggacacggggtgtgccagggctcctgggtctcttccacaatcccacaggcacagaacacaggcacggtctctcctcccagggcgtgtggtggcttctgactacgcagcagttgaccaagggtcaatctttgacaccaggtggcctagcgctgtcctagagggcagggccatgaacacacactagccactggcaacttcaaaggaagtgaacggaggctttgaagcctgcaaccccacaatcataggctctttcccccactttttttgtttgagacagggccttgctctggtttttttttttgttgtttttataatttttgcctttttccacatgttctgagaggaccttgctttgtcactcaggctggagttcagtggcaatcacagctcactgcagcctcgaactcctagcctcaagtgatcctcccacttcagcctcccatagcgttgggattacaggtgtgagcccctgcggctggccccagcccacagcctctttctgatacgagtgtctggtggcatcggagcagggtggactggccctgaggctcctgccaggccctgggtctgggcctgtggctgcagctcacctgctctttggtactggtcatctgcaggcgggtgcagaggtcatccaggcgttctcgctgctcatgttgccctaggcgctccagatactccctcagcatatggatgatctggaacaagagggtgagctgagctgcctgggttcctagggaggcagacaggctgatgctgcttccaaaaagcacagtggagcaggtgccaactggggtcagtgtgaaacaacccaggagccctgggaccagaggcaggtgtgtctgtggttagggcagcatctgtccagtgctgccttggtgccgtggtctccacacagaaggggtactcacctgcttcacctccagacgcacagcctctaggcactgggcatggccctcctgcaggatattcagctttgacttggccaggtgcaggggtgtgcgaccagctcggtccagggcatctacacgggcccctgcaggtaccagctgaggtgagcattctgggggtgagggggagaaagaggacagagaggggaagaaaccacatacctcctcgtagcagtgtggtgatgacaggaacgtggttggtgcaggccgctgaggaagaacaacagagacttaagaatccaggaccaccagagcctggctctgcgtggccactggagggagcagggctgtgtctcccagacgcctgtgccaagttatgcctactgagaaggtatcgccaaggcacagctaggtaggactcagaagtcaagttcaactccctccctgtacagcagaggaaactgaggcctggagataggatctgcctcagtgtgagccacagtgctgagtgctgctgggctagcctaggttgctaaattggctctgccgagggcacagccaactgctaaatgattggaaggcccatgggacacaggctgctgggcagtgcagccctgggagtgcaggcctaggatcttccctgacactcacagtttctgccaggagcatgccgggccccaccctcttcatctaaagagcctttctttgctgcaaatgagttgtcttagtgacttctgacatgcttacccaggtgcagtggcgtgttccccagcccatctcgctggttaggatcagcaccatggtccaggagcagctgcactggaaacaggcaaacccaagaggagttgtcggaggcttccattgcctgcaccctgaacccctaacctagctagagaggctggctcagagtaggggacacggaatgcaagcatggatggctgtgttacttcattcattcactcactcactcacttacttaccagagttcatgggcaaagccctgagggccaggaactgaggatttggggagggcctcttggcctatgcaggtgtaggaatgagatttgaagggttaagtaaagttcttccaatagcagctgctcctggtgaaggctgtccagggcattttctgcttctcccacagccaagcaccatggtaggaattaagtcaataaatgatgctgagctccatggataacctctccctcctggcagtcttgggatcacctcaggggtgtgctgtattatcaccagggtctgccaatacccagaaggatgcataggcaggtagcctggatcccagacatcatacagggaggtcaggctgagcctcacaccatgtacaaaaactaattcaagagggaccatagacataagtgtgaatgctaaaacaggcttccagaagaacatgaacatcttgggcaggcaaagatttcttaaataggtcacaaaaatactaaccataggaaagaaaaggccaggcatgggagctcaggcctgttaatcccagcactttgggatgccgaggcaggtggatcatttgagctcaggagttcaagaccagcctgggcaacatggagaaaccctttctctatactaaaaacacaaaaattagctgggcatagtgatggcagacttgtagtcccagctacttgagaggctgaggtggcagggcggcttgagcccagaaggtcgaggttgcagtcagccgagatggtgccagtgcactccagcctaggcaacagagacagaccctgtctcaaaaaaaaaaaaaaaaaaaaaaaaaaaaaaagagaaaggaaagaaatattgataaattggattcattaaaattaagaacatggctgggcacggtggctcatgcctgtaatcccagcactttgggaggccaaggcagccagatcatctgaggtcaggagttcaagaccaggctgttcaacatgttgaaactgtctgtactaaaaacacaaaaattagctgggtgtggtggcatacccctgtagtctcagctacttgggaggctgaggcaggagaattgctggaacctaggaggtggaggttccagggagccaagatcatgccactgaactccagcctgggtgacagagcgagactctgtctccaaaaaataaaaaataaaataggccagggcacaggagctcacgcctgtaatcccagcactttgggaggccaaggcgggtggatcatcggaggtcaggagttcgagaccagcctgaccaacatggtgaaatcccatctctactaaaaataaaaaattaggtgggtgtggtgacacgcatctgtaatcccagctactcaggaggctgaggtgggagaatagcttgaacctggggagcagagcttgcagtgagccaagatcacaccactgcactccagcctaggcaacagagtgagactctgtctcaaataaataaataaataaataaaaataaaattaggaacttgtgttcatcaaagtataccactcagaaaatgaaacagcaagctcagaaaatcaagtcatttatatttgataaatgatttctaaccagaatatacaaagaattcttacaaatcaataagaaaaaggccaggcgcagtggctcatgcttgtaatcccaatactttgggaggccaaggtgacaggatcgcctgagcccaagagttagagaacagctgggtaacatagcaggaccacgtctctacaagatgttttaaaattagctgggcttggtggtgtgcacctgtcgtcccagctacttgggaggctgaggcaggaggattgcttgagcctaggagattgaggctgcagtgggcagtgatcaggccactgcacgtaagcctggatgacagagtgagactttgtctcaaaaaaagaaaaagacagaaaactagttttttttgtttgtttgtttgtttttgagacagagtcttgctttctctcccagtctggagtgcagtcacgcgatctcggctcactgtaagctccgcctcccggcttcacaccattctcctgcctcagcgagtagctggaactacaggcacccgccaccacacccggctaagtttttgtatatttagtagagatggggtttcaccgtgttagccaggatggtctcgatctcctgacctcatgatccacccgcctcggcctcccaaagtgctgggattacaggcgtgagataccgcgcccaggcaaaacttgtttttaaaaaaatggacaaaagaggccaggtgcggtggctcacgcctgtaatcccagcgctatgggaggccgaggcgggtggatcacctgaggtcaggagttcgagaccagcctgaccaacatggagaaacccggtctccactaaaaatacaaaattagccgggcgtagtggcgcatgcctgtaatcccagctacttgggaggatgaggcaggagaatcactttaacccgggaggcagaggttgtggttagccgagatcacgccattgcactccagcctgggcaacaaagagtgaaactctgtctcaaaaaaaaaaaaaaaaaaaaaaaaaggacaaaagatttgaacatatagttcataaaagaggatgtaaaaatggtcaataattggacacagggaggggaacatcacataccagggcctgtcggggggtgggggccaaggggagggagagcattaggacaaatacctaatgcatgaggggcttaaaacctagatgacgggttgatgggtacagaaaaccaccacggcacaggtgtacctatgtaacgaacctgcacgttctgcacatgcatcccagaacttaaagtttaaaaaaaaaaatggcccgggcatggtggctcacacctgtaatcccagcactttgggaggccgagacaggtggatctcctgaggtcaggagtttgtgactagcctggccaacatggtgaaaccctgtctctactaaaaatacaaaaattaactgggtgtgatgatgcgtgcctataatcccagctacccgggaggctgaagcaggagaattactcaaacccaggaggtggaggttgcaatgagccgagatcacgccattgcactccagcctgggcgacaggcaagactccgtctcaaaaaaaaaaaaaaaaggccaataaacacatgaagaggtgctcaacgttactggccatcagagaaatgcaaattaaaccactatgagacaccaaaagggttaagattaaagagagacaacagcaagcactggcagggacgtagggaaaggtggaactcttacactgctggtggggagtgggagtgtgtgtcagtacatcccttttgggaaaaggtttagtagtacctactaaagctaaatacatgtgtaccctagtgacccaacaatgccattcctaggtttatagaggatagatgagtgtttacagccacaaatcacatatacaagaatgttcacaacacttcattcataaagctaaaacctggaaacaccacaaatgtccgtcaatagtagaatggataaatggggtatatcacaatgaaatacaaaccagcataagaaagtactaactacagtccaccctccctaactgtgaatactgtgtccacagattcaatgaaccctggactcagaacccgaggatatggagggccaacttcaaaggacttgagcatccatggagtttggtatctgagtggggtcctggaaccaatcccaagaacactgagggaagactgtactgatataggcagtattcatgggtgaatctcatggagataactttgagtaaaagtatgcaaggctggtgcagtggctcacgcctgtaatcccagcactttgggaggccaaggcaggtggatcacgaggtcaggagttcaagaccagcctggccaacatggtgaaaccccatctctactaaaaaatacaaaaaaattaaccggtgtggccgggtgcagtctctcatgcctgtaatcccagcactttgggaagccgaggcgggtggatcacgaggtcaggagatcgagaccatactggctaatacagtgaaaccccgtctctactaaaaacatataaaaaattagccaggcatggtggcgggtgcctgtagtcccagctacttgggaggctgaggcaggagaatggcgtgaacccgggaggcagagcttacagtgaaccgagatggcaccactgcactccagcctgggcgacagagcgagactccatctcaaaaaaaaaaaagactcctataaaaagagaaaagctgtcctgctgctgtggttggcactgagtaaatgttacctgaaagtcttgaaggcctgggccctgcactctccagtggtctcttagttggagatatcccttcatcctcagtcaatcatattatctcccccaggtttaccctgccccagtctaacattgccagttccacaggctaacagagggagaagggcagggcactgcagtgccaagtcttccttatatccagaacaactgggcatcccaaggctgtgcagacagcagcgcctcccccaaggcctgtgcagagacactacttgcctagccctactccctcctccctccccaggactcaccaatctggtcattgccattgcatgaggcaaagtgtagagctgtgcggcccttgtcatcagctgcacagggatccgcgccatcttccagcagctgctgcactgacaccaccaagacagagggaggaagggggagaaagcggcgatgttagctaagctgcagacccgagcgatggagagggaggctcaggggtgcctctagacaagacatcgacaatgcataggaaagcagcccttcctagatgctggtcctatgggaacaagcaacttgttcttctgagggcacaactgaaggccatggattctgagtgctcaaactacacgtctagtgatagaacatcgtaggaggttcaaggccctcagggtcagaaattttagggtcacaggaatccctagcatagacagtggtccccctcccgtgacctcggcttggatacacatccattctggctggaaacagctgcccaagctattggcctatgctatgctcccagcaggcagcaatcccccagctacttggggcctcccaagtagcccccagctaatttttgtatttttagtagagacggggtttcaccatattggtcaggctggtctcgacctcctgaccttaggtgatccgcccacttcagcctcccaaagtgctgggattacaggcgtgagccaccacacccggcccgaaaaaatgtttatgatacacaaaaaattatgcaggctacagcatgtgtatatagcagtttgcaagccagcccataaagaaaattccagtgtataggaaagaagccttgaaggatattcatcaaaatattaacagattagttcctttgcggacatggattttttcttatatatttctctaatttccacaacatttttaaaaaatttttttcttatgttatttatttatttgagacagaatttcgctctgtcgcgcaggctagagtgcagtggcgtcatctccgctcactgcaagctctgtctcccaggttcacaccattctcctgccgcagcctcccaagtagctggcactacaggcgcccaccaccacgtccggctaattttttgtattttcagtagagatggggtttcacagtgttagccaggatggtctcgatctcctaacctcgtgatctgcccagctcggcctcccaaagtgctgggattacaggcgtgagccaccgcgcccagctaacatttactttttgtagagacgaggtctccctatgttgtccaggctggtctcaaactcctgagcccaagtgatcctcttgccttagcctcccaaagtgctgggattacaggcatgagccacctcacctggccgtggcagctaagttgagtattcactcccattaaccacaagttccaaagttagtgcctactctttgcagaagactgtgctggctgccagggatacaaaagtatacatggtctccaccatcaaggagtccacagggtggtaaatgctatgacatcaggaagttcaaagtgacaggggagcatggggagagtgagtgtctaagccagacttaccttcagcaagatgactgcacagtcagacaataaatacagtgagcctgctccaagggcgtgagtttgcagactgccttcccacatattgattacctggccttttccttccccaaccagcccatgggaccctgtagctggacctactgtgcacactcacctgtttccacatcattggcattggccgagtccctcagtctcttcagagctgtaaagagagtaacggatgccagtttaaaaaaacagccatggctgggtgcggtggctcatgcctataatcccagcactctgggaggccaaggcaggcagatcacaaggtcaggagatcgagaccatcctggctaacatggtgaaaccccatctctactaaaaatacaaaaaaattagccgggcgtggtggcaggtgcctgtagtcccagctactcgggaggctgaggcgggtgaatggcgtgaacctgggaggtggagcttgcagtgagcagagatcgcgccactgcactccagcctgggtgacagagtgagactccgtctcaaaacaaacaaacaaacaaacacacacaaaaaaaacacagccattggctgggcatgatggctcacgtctgtaatcccagcactttgggaggccgaggcaggtggatcacttgaggccaggagttcaaaaccagcctggacaacatggtgaaaccccatctctactaaaaatacaaaaaaattagccgggtgtggtgcacatgcctgtaatcccagctactctggaggctgaggtaggagaatcacttgaacccaggacatggaggttgcagtaagccaaaatcgcgccactgcactccagcctgggtgacagagtgagacgctgtctcagaaaaaaacaaaaacaacaaaaaccaacaaaaaaaacccagccatctagctgggtgtggtggtgtgcacgtgtaacaccagctacttaggaggccgaggtaggagaatgggttgaagccggaaggcggaggctacagtgagatgagatcacgccactgcattccagcctgggcaacaaagtgagactgtctcaggaaaaaaaaaaaaaaaaaaagccacaatggctgggcgcagtggctcatgcctataatcccggtactttgggaggccaaggcaggtggatcacttgaggccaggagttcaagaccagcctggccaacatgctgaaaccccatctctactaaaaatacaaaactagccatacgtggtgactcacgcctgtaatcccagaacgttggaaagctgaggtgggcagattgcctgtgctctggagtttgaaaccaccctgggcaacatggtgaaaccttgtctatactaaaatacaaaaaattagttgggcatgggggtgcacgcctacaatcccagctacttgggaggctcaggcaggagaatcgcttaagcccaggaggcagaggttgcagtgagccaagactgtgccactgtactccagctcgggcaacagagtgagactctgtctcacacaaacacacacacacacacacacacacacaaaaaacataggccgggcacagtggctcacgcctgtaatcccaacactttgagaggccaaggtgggtggatcacgaggtcaggagtttgagaccagcctggccaataaggtgaaatcccttctctactaaaaatacaaaaattagccaggcatggtggtgcatgcctgttatcccaactactcaggaggctaaggcaggagaatcacttgaacttgagagccggaggttgcagtgagccaagatcgcgccactgcactccagcctgggcaatagagcaagactctgtctcaaaataaataaataaataaataaataaataaataaataaataaataaataaaataaaaacacaaaaattagccgggtgtggtggtgcatgcctgtaatcccagctacttgggaggctgagacacgagaatcacttgagcccaaaatcacaccatggcactccagcctgggtggcagagcaacactccatctcaaaaaaaaagaaagaaaaaatatttctcaatcttaggttaagttctaacttaaatcaatctaacatggtaaaaccccatctctactaaaaatacaaaaactagctgggtgtggtggtgcgggcctgtagtcccagctacttgggaggctgaggcagtgagccgagattgcaccacctgcactgcagcctgggtggtaaagcgagactctcgtctcaaaaaaagaaaagaaagacagtgccctgggaacaaggagaaacttgaaatgaggcggagctagtacaaaggccacatcctcctgcaaatcctccaagacttttggagccagatttcctctctatcacttgtagtcacagggtgaccttgagcaagctagttaacccagctgggcctcaggtctatcatctgcaaaacaaggctgagttgttctgccagccccgagttattgcaaatgatttacacagaagctctttgtgagctggttacacagataatcttatattctccttcaaagcaggtcgttcccaaatgatttgcctaaatttggtatttcctaagcagattttccccctccaatggctaatggaaagcaatgtctttgtcatcggagatatggtcaaaaattgcaattcagatacatgacaattccttgctttcatacagcactttatggtcaatacaacgcttccaaattcttatttcatcttcgtgacatcactaaaatatggctagagtcatagccagtcctggaagaaatccccaaagtccccattcttaatctctcagtgcctgggtttccatgtcacccatgcattgtagaagacaatatggtagatataccagaggagaagtttgagtcgggctgacccggattctaataccagcaacagccacttgctatgtgacctggaataagacaatttctctgagcctcagtttcctcttcagtaaagtgggaacaatttctatgtctcaaagagttcttgtgaggacagatgatcatgtatcagcagtgtttggggcacagcaaaggcagcataacagctaaacatccattaccttacttaactcatcccattttacagatgagaaaactagggctgaggccgggccagtggctcatgcctgttatgccagtactttgggaggccgaggtgggtggatcacctgagatcagaagttcgagaccagcctggcaaacatggtgaaaccccatctttactaaaattacaataattagccgggcttggtagtgggcgcctgtaaccccagctactcgggaggctgaggcaggagaatcgcttgaacccggggggcggaggttgcagtgaaccaagatggcgccactgcactccagcctgggcaacagagcgagactccgtctccaaaaagataataataataaataaataaaaataaaaaaatttaaaaagaaaactaaggttgaaagggtctatcatttgccctaaggtctaactgtggtaaaggagtccaagtccgtgcatttaaacaagctggtacgacttggctgcaggctctggcctcccctctccatgataataaaagtgttccccagtacctcattttattattcaccaataagcgttccgagtcatgggaagtttccattcagaaattacccagtgagagaagactggggtgactggctcagagcaggaagactttctaaaaaaaccatcatcaccatcgttactgaaaggagactcaagggtcagtgtggccctcagcgcagacgtcgggggaccccggctgagggtcgggggcgaccaaagcgcggccactggtcctctgaggccccgcccctcgcccgggctgccccgccccgggcccccgcgccgctcgcgccgctcaccgtgcacctccttgcccgtgggcccgagccgccggtggggcctggcagcgcgcctcagccggccagcgggtatcttgcagcgcagctcgtcgcgcggctccgcatcctgctgccacaggacgtgcaagtagcgcagcggcgactgggccccgccggacgcccggcccgagaggcccgcgccgccgccgcccagcgcagacccgaagtcagcgaaggagaagaggccctcagcgtcagtcagcggctccggcgccaccgcgcactcgccctccgagctcgagtggcctgagcgcggctcgtcgtccgcgtccccggcggcggctgccatggcaacggctccgcgcgggcctggccgcctgagcctcgttcccgaccgaccaacggacctacttccctccgccctgagtcgtgctgtcagcgagctggcgggcgggcagggcccgcaaccacggcaaccgagcgggggcgggctcaggccgagacagagcggcgaaccaatgactacccttccagcttaaaccggtcccaaaaccaatcggaaagggagggcacagtacaagcaggataattggacagggtttccggcaacgtgactgacagacattgtaaccaattacaaacttgaagtgaccttggctttcgctgtacgtcgctaccggagccagtaaaacagcatttcagacgctttttttttgcgacggagtctcgctctgccgccaggctcgcgatctcggctcactgcaacctccgcctccttgaagcaattctcctgcctcagcctcccgagtagctgggactacaggcgcgcgccaccacgcccagctaatttttgtatttttagtagagacggggtttcaccatgttggccaggatggtctcgatctcttgatctcgagatgtgccggcctcggcctcccaaagtgctgggattacaggcgtgagccaccgcgcccggcctttttttttctttttcctctccatttcccagtctaatgagaaaacaagcagataccttagcaaatcagattaagatgatttgcaggcactcacaaaggaaaatgaagaagccacgccatccatgtagatatagatgttgtaaatcaatgaacgcaaaaaaaaaaaaaaaaaagaacttcccacaaatgcataatgtacgtgggaaagggaaaatgtgttgaaatttggagataccactacaaactgtgagtaatagtgtctaaatgcaatggatttcctttagtcagagttctctaggtaaccccgacgtagatgactgatctacaaagatttgtaatacaaagtgattacattagagcaatagtttagttcgacagccagtcactgagccaggtaactgctaggtcctggacacaggatcagacttggtgcccctgagatgctcacaatccaatgggagaagaccaggaaacaaataagtgttcctcaagcaacatttcatgggtgcacgttttcataaaacttctcacaatcccgtaccccacaagcacttctataaagtagacaagaaggaccatccctattttataggtgaggagttgaaaagcagagggaagagactcattcacagttgcccagctctcaccggaccaggtcttgtgtcctggcaaaagttctttccatatataaaccctcccactcaacccagttaaaatgtaatctttccgcacggtgggtcacgcctgtaatcccagcactttgggaggccaaaatgggcagatgacaaggtcgggagttcaagacaagcctggccaacatgatgaaacctcgtttctactaaaaatacaaaaattagctgggtgtggtggcgcacgcctgtaatcccagctattcaggacactgaggcaggagagtagcttgaacctgggaggcagaggttgcggtgagctgagatcgcaccatggcactccagcctgggtgacagagcgagactccatctcagaaagaaacaaaaacaaaaaaaaggcctggcgctgtggctcacgcctgtaatcccagcattttgggaggctgaggcaggcagatcacgaggtcaggagattgagaccattctggctaacatggtgaaaccccgtctctactaaaaatacaaaaaaattagcctggtgtggtggtgggcacctttagtcccagctactcaggaggctgaggcaggagaatggcgtgaacccaggaggcagagcttgcggtgagccgagatcgcaccactgcactccagtctgggcgacagagaaagactccatctcaaaaagaaaaaaaaaagtaatctttctcttacaattggaaaagcacttccagaattttcagtgtttgcagtcgccattgtgaggtttatcattgtactatatggaaaaaaatgctatgtttatttgtactctcctcgtctgccccataatggatacagtgctgagacaggatctctcactgtcactcaggctggagtgcagtggtgtgatcacagctcactgcactcaagctttcctcccacctcagcctcctgagtagctggggccacagatgtgtaccaccgtgcctggctaattatttttttgattttttgtagacatggtctcacttttttgttcaggctggtcttgaactcctcgcctcaagggatcctcctgcctccacatcccaaaatgctgagattacaggagtgagccacagtgcctgggccccaatttttttttaagatggggtctcactatattgcccaggctgatcttgagctcctgagctcaagtgatcctcccaccttggcctcccaaagtgctggcatgagccaccacatccagccccatgaaaatcttctgacttcacaactccttctcaaatgtcatgaacattttgagaattgttgtcaaattggagcaacctgtatcaaattcctgtcatatatggactataagatttcaggtaatttcttttcttttttttttttttgagacggagtctcgctctgtcgcccaggctggagtgcagtggcaggatctcggctcactgcaagctccgcctcccgggttcacgccattctcctgcctcagcctcccaagtagctgggactacaggcgcccgccactacgcccggctaattttttgtatttttagtagagacggggtttcaccattttagccgggatggtctcgatctcctgacctcatgatccacccgcctcggcctcccaaagtgctgggattacaggcgtgagccaccgcgcccggccaatttcttttcttttttctttttttttttgaggcagttttgctcttgttgcccaggctggagtgcagtggtgcaatcttggctcaccacaacctccgcctcctaggttcaagtgattctcctgcctcagcctcccgagtagctgggactacaggcatatgccaccatgcccggctaattttatatttttagtagagacggggtttcactatgttggccaggctggtctcgaactcctgacctcaggtgatccatctgccttggcctcccaaagtgctgggattacaggtgtgaaccactgtgccaggcctcggagaagggttattatttaaactagaaactaaatgtctcccaaagttagctcggttcgagccaggaatgattaagggcagtctgaagattaaatggaagatgagggttggttagatcagatctctttcactgtcattattttctcactgttacaatttttgcaaaggtggtttcagcaataaaaatatttcacaaattttacaaaagcaaaggaccatgaggacattgttggggaccctccctgaaccaaggaagagactgtgactcttccttggttcagggaaggtcctgaaccaaatcgagggtcggtaaagcttaagctccattatttccaaatttgtgtcacctctggtgacagcacaaggaagaaacacaaaacaagaacgcagcatagggagttggggctgggcattgcaattctcaataggatgttgagtggatggcatcagagcagacacctgaaggtgccatgcagataccgatggggaagggaatgggagtgggggtgccactgcattcgattctagacttggataacagtgagtgcaaaatcccagaggtggagcgtgcctcagtgggaaaagcaaggagggcggcgtgacaaagggacccagagagtcctggtcacacagcaaatcagtggcggagtagggactggaactcggatttccactaggtgttgtgtatgggcagatggctaaactaaggcctctccttgactcatacaccggtttttattggtttgctttataaccccatctctaccttgagggtttcccctggcctcgaacccggtgtactcaagagctgcccatgtcccaggcttcgcgacacagctggcctcatctttgcctgctccaactttaatgtccaaagttgggtggctcctgttctccattccctcttactgcatgggaccaaatctcaatttttttttttctttttttttttagacggagtcttgctctgtcgcccaggctggagtgcagtggcgcgatctcggctcactacagcctctgcctctcaggttccaccgattctcctgcttcagcctcctgagtagctgggattacaggcggacgctaccactcccggctaatttttgtatttttaatagagacggggtttcgccatttcggccaggctggtctcgaactcctgatctcagatcatccacccgcctcggcctcccaaagtgtagggattacaggcgtgagccatcgcgcccggccaaatctcaatttttatccccagtaagtttagtcttgtgaagtccgaacgtttgaataatttactcgctgcaggcaaaccgcctacaactaaatccatcaggcccccgtatccgaatcttccttcacgcgagaagccggccatcttttgtgtagcattctttctggttcctctaactttgcctctcctttacgccaaatacgtgtccttatatctgaatctacaccactattatagattcaccttctttgagaagtccctatttgaagagaggaaaattcaggctacttgtaaccctcttattctttttagacggagttttccctcttgttgcccaggctagagtgcaatggcgcgatctcggctcaccgcaacctccgcctcccgggttcaagcgactctcctgcctcagcctcccgagtagctggaattacaggcatgcgccaccactcccggctaattttgtatttttagtagagacggggtttctccatattggtcaggctggtctcaaacccccggcctcagcctcaggtgatccgcccgcctcggcctcccaaactggtggggttacaggtgtgagccacggaacgcggctcctcttccttctggatatggctgtctcagatggccagaggcctcggaaaggcttccggtttcccggcaggcccgagtggggctgaacttccggcctcaggacgcaggcgcgggccgctcatttcgctctttccggcggtgctcgcaagcgaggcagccatgtcttatcccgctgatgattatgagtctgaggtaaggtggccgtaagggcgcggtgggctccacgacggggtgatctctgggaccactggatgtgggtcccggcgcgaggcagcttccgggtcgcggccagctccggccgacctcccgcccggccctcaggctgctcccacagttcccggtccctagacaaccctggctctgcccgcccacttcgcgtgttcgattggcttgtccttcccctttctaagaccggagactaaggcctgttagacgtttctgtgtttctgcacctgagtttttttggtctaggctccgtctggtcaaatctcgctgattcccacagcctcttgtcctttgctccacctcattgcacccgtaatgttgtattgtcattgtctgtttactgtcccttttctctaaggcgtgtgagttcctcaaaggcaggcacagtgtcttattcgcctctgtatccttagctctctgcttggcatctagactctaggatgagcgttttgaattcacgacttggcggctgtccttgactgtgtctctttccttctaggcggcttatgacccctacgcttatcccagcgactatgatatgcacacaggtgagaccacgggttaggctggctgagtactcgtagggatcagttcctttggaatgtcttagaaccagctctgacaggcatcaaaaattgctctttgatggtccaggtttggagtcagtcattagctgccagtgcgaggggttttataaagcctaataaaccaggctttttatcatagtccacctcagtcttggaagagaaggattacttacctggctatggttttcttgtgtaaactattagccaaataagaaaagcttttttgtttttgtttttgtttttgtttttgagatggagtttccctctgtcccccaggctggagtgcagtggtgcgatctcggctcactgcaacctccgcgtccctcgttcaagcagtagttctcttgtctcagcctcctgagtagctgggattacaggcgcccaccaccattcccagctaatttttaaatttttttaatagagacgaggtttcgccgtgttgtccaggctggtcttgaactcctgacctcaggtgatccacccgccccaacctcccaaagtgctgggattacaggcgtgagccaccgcgcctgccccaaggaagaaaatcttgatgtgaaaaatttaagtaattcaaaccaagcccgaatgaaatctttttaggaggtgtctgaggtttcctgaaacgttatagtgacttgacttagtagtcagtgttatccattgttctaagtactttgtatgaaatattttgtttaatccttacagcagttctgtatgttaatgtctcctaaagaatagaaaatatagcagagaggtttattagtagtaacttggcacagtcatatatttaaaaggtgacagttattcattcaagaaatattaagtacctgttaccacttactttccaacagtcttgcctctctcccctccaacgcacacttttctaggccctgagggtatgtctgagtacaagacagacgagattcctgacttcctagacttgggagaagggtgcaaaacagataataagtgaaaaataactttaagtcatggtaatatttttgaagaaacagggacaacgagattgggagtgacttgagatgtttgtatctctgagtattcaaaagggaattttacgcagacctgagtttagtttctggggtggtcttgccatttcgttctatcatatatgtgggtttgagcatactctgtatttgttgtatccaacctccaggagatccaaagcaggaccttgcttatgaacgtcagtatgaacagcaaacctatcaggtgatccctgaggtgatcaaaaacttcatccagtatttccacaaaactgtctcagatttgattgaccagaaagtgtatgagctacaggccagtcgtgtctccagtgatgtcattgaccagaaggtgtatgagatccaggacatctatgagaacaggtatgggctactggctgaaagggcctctttctgggtgggactggcctggtaatatagttcctacaaatgaggtgagcactctgtaaacagtactttcgggggggttggggggtgggggtctttgtgagtccttgaggctagggacattttattttgttagactttattttatttatgcatttgtttattttttgaaacggagtgtcactctgtcgcccaggctggagtgcagtggcaagatctcggctcactgcaacctccgcctcctgggttcaagtgattctccagcctcagcttcccgagtagctgggactacaggcatgtgccaccatgcctagttaatttttttgtatttttagtagagacagaatttcaccatgttggccaggcgtgtgttgaacccctgacctcaagtgatccacccgcctcagcctcccaaagcactgggattacaggtgggagccaccatgcctggcctaatttttgtatttttagtagagacggggtttcatcatgttggccaggctggtcttgaacttctgacctcaagtgatgtacccccgttggcctcccagagtgctaggattacaagcgtgagccaccatgcccggcctgttggactttatatcctcagaaatttatacaaagcctaaaataatatcactattaacaattataataataatgattagcatttgtttttattaccaggatagatccatcaatgttcagtgtgttgtttgtgtttttactcatttacccttcacagtaagcccatgaagtagcagtttttctcattttacagatgaggaaacagcacagaaataagtcatgtagctagtttaatggatgtgctgagatttgaacctgcagtgtctggcaccaaagcctaatttctaaactgtgctatgcagcccttcagtacatatcagagcaagctgaactccgcaggttttgaggggctgccgttgtgtaatgagcgttagagataagataggtcagtcaaagctgggatagtcaggagtgacttcaccaaggaaatagaatttaagcaagtcagaaggatgagggagagaaaacaaatagccaaaggcctattgatcaatgaataggatctctgacatgtgttttctttttttgttgttttgaaagagctgattgtagttgaaaagcagaggaaacctcagacattgcatggttttaggcctcacccctgccactctcgtgagcctaagttctacttggtgtggactccaaggtccttcccgcccctgcaagccacccattaggttgagctgagagatcagccaatgggaacagagtcacttatgaccaagaagaattgatctgggggttagttagtgaggagagagaaagggagaaaaccttgttcggaggttgaacacctccagccgaagaaggtgcagcatcgaggtctttcaccactagcaaatggatccaagtcatgcggcaccaaaagaggttagcagctgaagggtgttgaagtgacacaacaccaaaacatgctaccaatgatgaatccgtatctgtacaggtctccagcagcctcagttcttgcctcctcaaaagaaagaattcgactgaggggtaaaatgctaccgatgatgagtccgtatctgtacaggtctacagcagcctcagttcttgcctcctcacaagaaagaattacactgagggctaaaatgctactgatgatgagtctgtatctgtacgggtcgacagcatcctcagttcttgactcctaacaagaaagaattcgactgaggggcacaaggcagaagaagagagggaggcaagtttgagagcaggagtgaaagtttattaaaaagctttagaacaggaatgaaagaaaggaaaggacagttggaagagggccaggtgggtgacctgaaaggcaagtgtgctgaactgtgttttctttattccccgggtcttttctgattagaagtatataaatcagcgcctacaaaaattagctggggcatggtggcagcatgcctatagtcccagctagttgggaggctgaggtgggaggatcacttgagcctgggaggttgaggctgcagtgagctgcacaatggagcgagaccctctctcaaaattaaaaaatggacctgatgttcaatagaaatgtagctagacattaattttaatttttcagccaagcgaagtggagatttatttaaaatattatttctatatgtaatcagtatttaaaaattaaggggatatttgcccttttttttctttatacttaaagtttcctaaatccagtgtgtatcagaaatacttgctttgtgtttagattttatagagtttatgactggaaaagtaattcaaatttgaaatttgaatcttcaaatagtagagtaattccaaatagacctaaaagttttccaatactgagttaagtgttggtttttaaacttaaatggcaactaactgaaatttaaaatttaactctttgcttatatagtagccacatgtagctagtggctattgcattgggcagtgcccgcagatacagatgacagttgaagccaagcatcttgtaagtctttctctaggagaatgtgagaagaaatgctggtgccttaagaagtatgtttctttagggatgggtgggcaggcaaagaaaggatctagtaaaggaggcagaaagaaaagtgaaaagtagatagtaaattggcaaagaaatgtcacaggaaccaaaaagtctgcagaagaaactgaggaagagagtatcaccaaatgttttagtgaggtccagatgagaaaaaaagccagttgaatttggtaacttggagggccttgataaccttagcaaggtcaatgtcagtagagtgatggaagcaacagccagagagcagtggggtgagaaaggggaattggtaagtatcggggacataaagggtcactgagtggctttgtgttttgtttctgtgttttttctgtttttgttttttgttttttgagacaggatttcgctcttgttgcccaggctagagtgcaatggcgcaatctcagctcatcgcaacctccgcctcctggctccaagcgattctcctgcctcagcttcccaagtagttgggattataggtgtgcaccaccatgcctggctaattttgtatttttagtggagacggggtttcaccgtgttggtcaggctggtctggaactcctaacctcaagtgatccacccgcctcagcctcccaaagtgttgagattacgggtgtgagccagcgcgcctggcctgtttcagtgtttcttttctgttctgtttctgtgtttctccccaacccccatcccgtagagacagggccttcctatattgtccaggcttgtcttgaattcctgggttcaagcgatacccctatcttgtcctcccaaagtgttgagattacaggcgtgagccatcatgcctggcctgttcagtgttttttaggggaacttgagcatgtttatatgttggactaaaagagccagagaagcaggaggggtggggcttgaggagattggaggaaagtattaatagctgctgagggcatcaggagaagaaaattaaacaacctgttgcaggtttgtcaagtagcagtgtttggattggataggcagcagtgtttaaattggatgctgtaggtctgttgtggccttggactgcagcagactgatttgcttctgtgtttagagacgtttggatgtgctccaggagatggaaacgctgtgttaaagaatggtggccaaaaaagtagttcttatagcagaaaacagcaagcagttaaatatggctatagttaatgatacagaggcaggagattatcctgaaaagcttgatgggtccctaactgatacaaaaacaatattttcttgtgttgagaaagattgaagtgtggagttgggttgaggacagttgtgggcttgaagggctttggcctcagctctgtttatttgacatcttcccctttctaagcctgggtcctcatctgtaaaatgttaggattggcattctccagtccttttggaattttagagatttttttcttgatttttagctggaccaagctgactgaaagattcttcaagaatacaccttggcccgaggctgaagccattgctccacaggttggcaatggtaggtgtgagctctttatatcttgtgcactgagtggaatccagtggctgagtcattagacaggaagctcaagagggtctgtgtctgttttgctcaccattttgtttccttggtgcttggtatgtagtaggtccttggcaaaataacttgtcgaaggctggatgatttaagagaggacagagatctctcatatatggtgaaatttagatctttgtactgacttgtttctatttccttctataatttgggggtcattacagtcactctggaataagctctctagcttcccacctttgactttcttagatgatgtcctttatatatgggtgaggtaggcaaatccaacttcattattttgcttgtggatattcagctaccctggcaccaatttatttatttatttattttattttattattattattttttttgagatggagtctcgttctgtctcccaggctgaagtacagtggcacaatcttggctcactgcaacctccgcctcccaggttcaagcgattctcctaggttcaagcaattctcctacctcagcctcccgcgtagctgggattacaggcgcccgccaccacgcccagctaatttttgtatttttagtagagacggggtttcaccgtgttggccaggctggtctcgaactgctgacgtcaggtgattcgcccacctcagcctcccaaagtgctaggattataggcatgagccaccgcacccagcctgcccagctaatttttaaaaattttttgtagataccgggtcacactttgttgacctggctgatcttgaactcctgacctcaagtgatcttcccagagtgctgagattacaggcatgcaccactgtgcccagccacattgtcttgattactatatttttgtagtaagttttgaaatggggaattgtaggccgggcgcagtggctcacgcctgtaatccaagcactttgggaggccgaggcgggtggatcacctgaggtcgggagttcgagaccagcctgattaacgtggagaaaccccatctctactaaaaatacaaaatcagccgggcatggtgacacatgcctgtaatcccagctacttgggaggtggaggcaggagaatcacttgaacccggaaggcagaggttgcggtgagccgagatcgcgccattgcactccagcctgggcaacaagagcgaaactccgcctcaaaaaaaaaaaaagaaagaaagaaattgggaattggtaagtcctccaactttgttctttttttttttttccccaagattgttttgactattctgggtctcttggatttgcttattgattttaggatggacttgtcaatttctgcaaagtagccagctgggattttgagaggaattgcctagaatctgaagatcactttggggagtattgccgtcttaacagtattaagtcctccaggccaggcgcggtggctcatgcctgtaatcccagcactttgggaggtgaaggtggacggatcacgaggtcaggagatcgagaccatcctggctaacacggtgaaaccccgtctctactaaaaatgcaaaaaagttatctgggcaggccggcatggtggcgggcacctgtagtcccagctactcgggaggctgagacaggagaatggcgtgaacccgggaggcagaacttgcagtgagctgagattgcgccactggactccagcctgggcgacagagcaagactgcgtcccaaaaaaaaaaaaaaaaaaaaaaaccaatattaagtcctccaatctatggacatgggatgtctttgatttatttaggtctttggttgcgttcaccagtgttttgtgttttcagtgtacaagtctctcatttgttttgttatagctaacgtttttttgaacacttaactccttttttttttccccctaagatggagtcacggagtcttgctgtcttgcccaggctggagtgcagtggcgcgatctctgctcgctgcaacctctgcctcctgggctcaagcaattctcctgcctcagcctcccgagtagctgggattacaggcacctggcggattttttgtatttttagtagagatggtgtttcaccatcttggccaggctgattttgaactcctgacctcaaatgatccacccgcctcagcctcccaaagtgctgggattacaggcatgagccactgcacctggcctgaacatttactctcaattgactaaacacagaatgagttcaaaactcattctgtggctgggtgtcgtagctcacggctgtaattctggtactttgggaagctgaggtgggaggatttcttgaggctgggagatttagactagcctgagcaacatagggagacccccttctctacaaaaaattttttaaaaaaattatccaggcatggtggcacatgcctgtagtcccacctactcgggaggctgaggtggaaggatcacttgagccctggaagtcgaggctgcagtgagccatgatcatatcactgcactccaaccttggcaacagagcgagaccctgtcttcaaacaaacaaacaacattctgaaattaatattttctttcttcctttttttttttttttttttttttttgacatgggggtctcactgtgttgcccatgctggagtgtagtgtgataatggctgactgcaacctgtgcctgctgggctcaagcgatcctcccaccttagcctgccgagtagctgggaccatgggtgcgcaccaccacgcttgtctaattactcttgtgtgctggtttttttatgcacttggatggcagaagtctctaaatggtgaaaggaaatagatctgaatgagatcttaatctcatcgacagagattgaggttgatggagtcaaatgagaagtggattttctcattgcatgtattcttggggcatatgcatagcaatttagatgttgtttagtcttcagaattacctttattcctccccaccaaagagctaaagctgccaggataccccagttttatggttagtgaagcacccttctgccatctctttcagatgctgtcttcctgattttatacaaagaattatactacaggcacatatatgccaaagtcagtgtaagtatttaagtgtcgcagttttttttttgtttttgtttttttaactctgtgctgtttttgtccctagaacacagtcatttgtttatattggttcagtgtctgggcatgattggcatgtttttgagttttaagaagcggtggaaggaacagtttaacccagaaagctgttcagacacactaacgctatataacctatgcattcttttaaatttctctgtccccacaattctcctggactgggcaatcacccatgcactggttataggctgccagcctcataggatatttccaggtacctgctgctgcttctgcttggggttctttttcccttcagagcattgagcctctgtttctctgagatgtacataacactgtcactgtatgccaagcacaatgctaggcacttagcacacatcttcacatatctaattctactatcaatcgtgcaaagttggtacccgaggtgccaagaggttaccttttctcaaatttatccagctagtaagtgacaaaactccttgaatctagaccagcttgttataacaccagtattctacacattatcccaactttgagcacttgctagattctgttcttcccgacttctaaattatagagtacgtgaagtttctttttttttttttttttttttgagagagagtgagtctcgctctgtcgcccaggctggagtacagtggcgcgatctcagctcactgcaggctccacctcccaggttcacgccattctcctgcctcagcctcctgagtagctgggattgcaggtgcctgccaccacacctggctaatttttttgtatttttagtagagacggggtttcactgtgttagccaggatggtctccatctcctgacctcgtgttccgcccgccttggcctcccaaagtgctgggatgacaggcgtgagccaccacgcccggcctagagtgcctaaagtttcttaaaatgccatctgtactatttaaggctgggcgcggtggctcacgcctataatcccagcactttgggaggctaagggcaggtcacctgaggtcaggaatttgagaccagcctggccaacatggagaaacctcgtctctattaaaaatatgaaactacaaaaataagctgggcgtggtggcgggtgcctgtaatcccagctacttggaaggctgaggcaggagaatcgcttgaacccgggaagcggaggttgcagtgagccgagatcgtaccactgcactccagcctgggtgacagagtgagactcttgtctcaaaaagaaaagaaaaatgccatctgtattatttatatacaccaatgacttaatctctatctttagcctcagttgccccactgaagtccagacttgtatattcagctgtcagcatctgcacttggctgtctgagtcatctgaagcttaacacatctaaaactgaattcttattttccctctaaactagtttttctcatggtaatcatctcaataaatgctaccgtttattaccaagttaattggactaaaagccttggcattattcttgacttttctctgacatgatatccagcaaatcctgttaagctctgtcttcaacatacttctttttgttgttttgttttattttgttttgtttttgagacggagtctcgctctgtcatccaggctggaatgcagtggcgccgtctcggctcactccaacctctgtctcctgggctcaagcaattctccaggctcaaacgattctccttcctcagcctcctgagtagctgggattacaggcatgcaccaccacgcctggctaatttttgtatttttagtagagacagggtttcgctatgttgcccaggctggtctcgaactcctgacctcaggtaatccgcccgcctcggcttcccaaagtgctaagattacaggcgtgagccactgtgcctggccttcagcatacttcttgaatcagccacttttcactgccacactgctgctcacctagttgaagtcattatcatctcttccctgaattactgcaggagctgcttacctggtgtcagtttccactcttggctgtttattctcaacataataaccagcatgatccttttgctacatagtttcatcaggtcacttccaatcctaccatgacttcccatcacacttactaattccaggttttagccctgtttcccttctcactcatttcctgaaactttccccctccctccctgggctcctggcaatattcgtggcacttcagagcacttatctcttgtacgttgtatttgtttatttaggggagggagtttcttaatctactggaaggttaactccaccggggtatgtgcttgtcttgttcaccaatataatccagtgcctagaataatgtctggtcataatggtgttaattgagcatgtgtgtgcttgcatgcatgtgtaagtgaaggaaattggcaccaagatctccacctaaggaggtcctagtttctcacagttactcacccctcttctccttgaggaccgcttgatatggctagaactcctgccttggccacaggagccactgtcagtagtggtaggtggaaaagttcttgctgccagagtaaggggccatgtttttccggcatcccagcgggatctctgactactttatctgatagaaagtaaagattcctggccaggcgctgtggcttacgcctgtaatcccagaactttgagaggctaaggcgggtggatcacgaggtcaggagttcgagaccagcctgaccaatatggtgaaaccccgtctctactaaaaatacaaaaattagccaggcgtggtggcgtgtgcctgtaatcccagctactcgggaggctgagacggaagaatcacttgaccccaggaggcagaggttgcagtgagctgagattgcaccgctgcactccagcctaggcaccagagcgagactccgtctcaaaaaaaaaaaaaagtaaagattccctactaccattgatgctaaagttcaggtactttgtggatatgctaagaatctagccattatgtgtacactgcttttttgggaacaccatgacaccatagcccatagtatagctggttttaccacctctaatctctctgtttggtctgtagatgaagattcagcagaagcttattgatgctgttatctttttattagctaccaggactcctacataaatgaccagttttaagcccttgggaagcagtttttcattgaatgtgcttagattgtgcttaatgtattaagcaccttacataacttttattaaacagagacggggtcttgccatgttgcccagcctgttctcaaactcctggcctcaggtgatcagcccacttcagcctcccagagtgttgagattacaggcgtgagccactgcgcccagctatataatcttacttcattgtgactagtcaacaatcttttgcccacattttacagagattaaataccttgtgagaaccccacagttaataagaggtgggactgactccaaagttgtgtttcttctccatcacaaatacattgctcatggtatggttccccagctcagaaactatcagtggttctcagatgcaagcaacataaagaccagaccaggggtcagccaccttctctgtggagggtcagataataagtattttagtctcgagggccatgatggtttctgtcagatctgttcagctatgtctttgtggcagttatcaacaatatgtaatgagtgggcatggctgtgttccagtaaaacttctttatttacaaaacaggtggcggaatagatttgcaggccccctgacctagatgcttgtttggcgttggaagactgcaacattcctggcccgtagtctagtgatgcccggtgcagcattatcttctcttaactccctagccttcaagtgccacgatttaacttcttcgcctttgctagtgtggtttttttctgaaacacttttccttctctctgctttgccagatcatcatccctgctctgagggtcatttaaggtctgttgctgtagaacctaaccacgtgttgcagtattcagtgcgtcagctgctataggactttctatcttccctctcacttgcatatttacatagtaagaaagagaggacagataccaattctcttgtatttgttcacagctgtcacagtataccatgggtaaaacacattaaaattaaatatctgtatcttgatatctcttgttttctttacaggggggaccttccttggagcagaggtttgaatcctattacaactactgcaatctcttcaactacattcttagtgagtctgagatttggccaagagggtgtgtgtggttatgatgaggttcagcattggcctccagcttttttttttttttttttaaattagcaggatcttaatgtgtaggtgggaagaggggaggtcttgtgattcatggtccttttaagagagaaattacaaagcattctgcagcctacagaatgggcagagtcattgctttgaatctgacttttctgtgatctcctgcacagatgccgatggtcctgctccccttgaactacccaaccagtggctctgggatattatcgatgagttcatctaccaggtatctggtcagcttcagcctaatttgaaataactggtccatgccaggaatagctgttactattgtttgtgtaaaaaagctgcagggtgtaaaaatatccccattgtagtgtaaaataccccatggcagtagactccagtttcagctggctttcggaagaggcagggaagcctaagcactccagataggaagctggaagtcggataagttcagctcctgaccttcatcaagctcttactagaccttgttaggtggtctggtggtaagaacacaggcttcacaggctttggagcgaggtggaagtggattctaacttgggtgctgccacctgctggtagctctgtaacagtgatgtgctatttaaactcagagcctcactttctttacccacacaatggaaatgttctattatctttttttttttaagagtcagggtcaggccgggcgcggtgactcacgcctgtaatcctagcactttgggaggcagaggcgggcagatcacgaggtcaggagatcaagaccatcctggctaacacggtgaaaccccgtctctactgaaagtacaaaaaattagccgggcgtggtggcaggcgcctgtagtcccagctactcagtaggctgaggcaggagaatggcgtgaacctaggaggcggagcttgcagtgagccaagatcgtgccactgcactccagcctggtaacagtgcaagactccatctcaaaaaaaaaaaagaaagagtcagggtctcactctgttgctcaggctagagtgcagtgataatagcttactgcagcctagaactcctgggttcaagtgattttcccacctcagccacctgagtagctgccactacaaggcccacaccaacttacgtggatatttatattactgtagagacgagggtcttgctgtgttgcccatgctggtgttgaactcctggcctcaagtgagcctcttgacttggcctcccaaagtgagccaccatgcctagccatctatatcttgattgtggtggtggttacttaagtgtatgctcgatcagaatacaatagaacattttcatcacaacaagaatatcacctacctcaggagttgctgttcaaggtcgaatgagaactgtgccaaatgcaaataaagtacagcagctgtgtagcctgggggtaaaagtccaggttttgctgttgctgtttactggctttttttttttttttggagatggagtctgactctgttacccaggctggagtgcagtggcattatcccggctcactgcaacctctgcctcccgggttcaagcgattctcttgtctcagcctccccagtagctgggattacaggtgcccaccaccacgcccagctaatttttgtatttttagtagagacggggtttctccatgttgaccaggctggtctcgaactcccaacttcaggtgatccgccctccttggcctcccaaagtgctgggattacaggtgtgagccaccgcgcctggcctactggcccattttaaaatggtaacatgttctgttgtgggcacattacttaaccacactgtgcattggtttccccatctctaaaacggattgctgcgaatgttgctgagatactacgtatagagtgtttagcatagtgcctgtcacagaataattaatacacgttgcctgttgattgtgatgatgttcaatgtcatcattattcttttccatcttatttagcccagtccttctatttaaaaatacagattgctcacaactgtaatcccagcatttttggggtccaaggcagtaggatcgcttcagtccaggagttcgagaaatagagactacaaaacataaaataggctgggcgtggtggctcacgcctgtaatcccaacactttgggaggcctaggtgggcagatcacgaggtctggagatggagaccatcctggccacaatggtgaaaccccatctctactaaaaaaaaaacagctgggcgtggtggtgtgcgcttgtaatcctagctactcgggaggctgaggcaggggaatcgctcgaacccgagaggcagagcttgcagtgagccaaaatcgtgccactgcactccagcctggcgacagagtgagactctgtctcaaaaaaaaaaccaaaaaaaataaaatatataatttgacaaaatataattcttttccccctttttgtgctgtctatagctctaactgattatttcaggataaattggtatcttaataaagatgttattagaactccgtgaaccagggagacgttgtgtatacaaagtgctgaaccgttacgagtggtgagctgagaaaccttcatagtgctccttgagaaggaggcttcctgctcaagattaggcttcagtgcctttcatagcaaaggccacaaaccgtactggccttgtttgccctctggctccttggcttgtgtcatccttttatttggaatatttatagatgaatagcagtctttttgacttagagcaagtgaagaaggatcatccgtgctgattttctttatccctcttaagataatcataattccctgggcttagccagtgttgttgttaaggggcttacggtgctttctaacaaagactgtccagtgagggtcagtaagtggctggtgtatttcgtcctggccaggtagatgccacatggggtaggtggctcttcctcgccaccctgttttatttatttgtttttgttttttcaaactacagatatagtgaaagtgcctgccttgcttgcttttgcagaggtatcaattactgtctggaaggaaaggcaccttgttgatgtttctcaggcacttacataaacatgactgtataaagagagatcacttcaaaaatggttacgtactctattgtgccatttactgtgacttgttaacacacttaaaccatattttcatagacctgacttgctcttttgctgcagtgtgcgtgatatggatataccatgcctctgatgatgaacctttgggttgtcagtttccacaattacaggcagtgaatcagtgaatatccatatatatgttgtacacaggcatatttttagggatcactacctagaagtagaattgtggattgaacgatacttgctttttaagtttttttaaatcagcttactgaggtatagtttatttttttaagtagagatgagccaccagctcgcgcctgttaatcccagctctttgggaagccaaggcgggtggatcacgaggtcaggagttcgagaccagcctgaccaacatggtgaaaccccgtctctactaaaaatacaaaaaaatcagccgggcgtggtggcaggtacctgtaatcccagctatttaggaggctgaggcaggagaatcgcttgaacctgggaggtggaggttgcagtgagctgagattgcgccactgcactccagcctgggtgacaaagcaagactctgtctcaaaaaaaacaaaaaacaaaaaacaggtgtgaaccaccatgcccagccatgaggtgtagtttaatatacaataaaatgtaccagaatcagtgtacagtggatgagattcaacaaatgtgtacagtgccacaatcaagataaaagaacgtttcaatcactttgaattataataaatagtaatatattgtcccaccaatgggctgtatcaaattgtgttactaccagcggtgtttgagagcaactgttggtttttttgtttctttgtttaagagatgggggtcttgcctttatggtccaggctggagtgcagtggtgtgatcatagctcattgcagccttgacctcctgggctcaagcaatccccccaccttagcctcctgagtagctcggactacaggtgcacccagctgatttttaatttgttgcagagatggggtcttgaaatcctgggataaagcaattctccctgctcagcctccaaaagtgctgggattacaggcgtgagccaccatgcctagccaagaccaactgttttctgtactggatattatcagtctgtctaatttttgtcagttcaatgggaaagaaattctggccgggcgcagtggctcacgcctgtaatcttagcactctgggaggccaaggcgggcagatcacttgaggccaggagttcgagaccagcctggccaacatggtgaaaccccgtctctactaaaaatacaaaaattacccagacttggtggtgcatgtctgtaatcccagctactcagcagctgagggatgagaatcacttgaaccgagaggcagaggttgcagtgagctgagatcacaccaccgcactccaccctgggcagtagagtgagattccgtctcaaaaaaaaaaaaaaaaaaatcagcgcggtggttcacacctgtaatcccagcactttgggaggccgaggcaggcggatcatgaggtcaagagattgagaccatcctggctaacacggtgaaaccccgtctctactaaaaatacaaaaaaatttgccaggcatggtggtgggcgcctgtagtcccagctactcaggaggctgaggcaggagaatggcgtgaacccaggaggcggagcttgcagagagccgagattgcaccactgcactccagcctcggagacagagcgagactccatctcaaaaaaaaaaaaaaaagaaaaagaaaaaaaaatcatacttttctgatttcccatattaattgtcaattttgttttctttgaagtgatcatcactattctttgcctgttttctatttgattgcttttttaattgcattacgagaattctttgtaggattttttttttttttttttttttgagacagagtctcgctctttcgcccaggctggagtgcagtggcatgatctctgctcactgcagtctccacctcctgtgttcaagcgattttcctgcctcagcttcccaagtatctggattacaggtgtgcaccaccacgccctgctaattttttttttttttttgcatttttagtagagacggggtttcaccatgttggccaggctggttttgaactcctgacctcaagggatccacccatctcggcctcccaaagtgcttggataacaggtgtaagcgccaccgcacccagcctgagaattctttatatattctggatattctttttttttcccccctaaggcggagtcttgctctgtcacctaggctggagtgcagtggcgtgatctcagctcactgcaacctctgcctcccaggttcaagggattctcctgcctcagcctcccgagtagctgggattacaggcatgtgccaccacacctggctatttttgttttggtgctttttttttttttttttttttttttgagacggagttttgctcttgttgcccaggctggagtgcaatggtgcgatcttggctcaccgcaacctctgcctcccaggttcaagcgattcacctgcctcagcctcccgagtagctgggattacaggcatgtatcactgtacctggctagttttgcattttttgtacagatggggtttctccatgttggtcaggctggtgtcaaactcccgacctcaggtaatccgcctgcatcggcctcccaaagtgctgggattacaggcatgagccaccgcacccggcctgtttttgtatttttagtagagatggggtttcaccatgttggccaagctgttctcgaactcttgatctcatgatccatccaccttggcttcccaaagtgctgggattataggcgtgagccaccgtgcctggcatattctttgtttttatattacaaaattagcattttgtcccacagtctagaagaagataattttttgtgttttgagacagagttttactctgtcactcacactggaatgcagtggcacaacctcacctcactgtagcctccgcctcctgggctcaggcaaagcccgtacctcagcctccttagtagcggagactgcaggtgcctgccaccattcctggctaattcgtttgtgttttttatagcgatagggttttgccatgtttcccaggctggtcttgagctcctggacccaagtgatctgcccacctcagtttcccaaagtgctgggtttacaggggtgagcctctgatctctgctggaagagttgaagaacagtcttagatcttctaagacctaggttttaggtctgtgatttccaaacctgatgcacaccagattcaaccagagaactttaaaaaattgagtaggtaggtctgttgtgttccatctgtgtgactgctggagccaggccaggagtgcttccaggttctacttctgtcccaaacagattcctgtggcatatcttctctgtgttcgttgtgcttggccagacgactctggttgagagtggtagagaggtgacatagtctgggaactgatggtcagctgtggtagggaaggctcacacgtgcagatacctattaccacatcactgcccataacccagggcgtaccacagtctagctgcatctgaatagatggtacaagagcccctgtggtgtttctggtggcccggtcaactgcattgtgaggcccagagacaggaagtgactttgggttgtgtttgactccagattggatgtttttaccacagactacctctaatcctagagatttttacttcactgtgtagtaagtaacattcagtaatgaacagaattcacagtttagtcaagatggctttgaaaaacagtgtacggaatggtttagaatgagaagtggtcatctcaccctcaccttgccccatccagagccagagcattgcctcgttttcagcattgtggacatggatgatcactttgggcttataccactactgcatgtttcttttcttcctcaacagtttcagtcattcagtcagtaccgctgtaagactgccaagaagtcagaggaggagattgactttcttcgttccaatcccaaaatctggaatgttcatagtgtcctcaatgtccttcattccctggtagacaaatccaacatcaaccgacagttggaggtatacacaagcggaggtgagtgcagcaggccgacagccgtggcctgcgaatttccagctgcttgccatctgttccaaatgaatagatcactgcaaggagagcagatgagtcaccatgtcttaggtggtggtctgttgactctttgtaggtgcttgagtacaaacagtagcagagtatttgtgctgggggctctggggcttgggagtccattggccaacagactgaaccagcacattgagatttgcgtttgtaaacacttctgtgtctccagtgcttttgtttagataagcacttctcacacgtgttggtgtcagagccactttaccctgttaaaatattgagaaccacaaaatgtatctattttaaaaaatttttttctttttttttcagtgaagagtggcatttttgcagatctctctgtctggctgagatagctggcttctcatttctgcttctgcattcaatctgttgcaatgtgttcttttgtttgaagcacataaagaaaatctggcccaggcactgtggctcacacctgtaatcccagcactttgggaggcttaggcagaaggatcgcttgagtccaggagtttgagaccagcctgggcaatatagtgagactttgtctcaaaaaaaatgtttaaattgactgagcatggtgggtggggtgcccctgtagtcccagctactcgggaggctgaggtgggaggattgcttgagcccaggaagcaaagattgcagtgaactgagatcacaccactgtactctagcctgggccacagagcgagaacttgtctgaaaaaaaaaaagaaggaaaatctggcttcacacggttataggaagtttttttgttgtttgtttttgagacagggtgtcgctgtgtcacccagtctggtcttggactcctgggctcaagcagtcctgccttggcctcccaaaagtgccaggattattggagaggattttctttgatactacaccatgtagtagtatgtatgggttgaatattcctgacttgaaaatttgaaatctgaaatgctccaaaatctgactttttgagcaccaacatgacgccgtaagtagaaaattctacacccaacctcatatgagcagttgcagtcagaacttcgtctcatgcacaaaagtatttaaaagattataataaagttactttcaggctgtgtgtataagacatatatgaaaaataaatgaattttatgtttagatttggatcctatccttaagatactcattagggccgggtgccatggctcacgcctattatcctggcattttgggaggctgaggcgggcagatcacctgaggtcag

SEQ ID NO: 7 - DNA, 33737 bp, Homo sapiens
cctcaattactacattactgattatacgacctttctgtggtcgcggtttattgagctattcctgcgcccacctgttttgattattttagctttatggtatgttcggtaactggtagggaaagaagcgcattgttaaatttccccaaaatagtcatgtctattatctgtcattcttagagttggactgcagaattggttctctaattttcaaaaatcattcttgtgttatgtggtaatatcacagcatatgggattaatttgagaactgcgatctttataatgctcagtgtttttgttcagagacatgttgtactctccattcactcagataagtggcttaatattttattcatgcaaatcttgcacactttgttttttattcataaagggtttgtaaatataattttattgaagttataaattttttcacaattttatatcgtaaatgattactgtttctatagcaaggaaccctattaacttttctatgttgcccttgtatccagacactttaactcttgtattaattccagcagttcttcagctgattctccgtgtgtgtgtgtgtgtttgtgtgtttagttaactatcacaccatttgccaagaacgattttctctctttttctgtaatatttatacctccttctctcccccttttatgtcatttcattggctggaatctatacaatatgctgaataataaaagtgagactagacaaccttgccttgtttctgattctttaaatgttttgcctttaaatatgaaggttgctgtaaatttggggagatattcttcactgagtcaagaaaattttcttcagtaacttaataaaaggctaaatgtttgctttctttatatgagaaacaagtgttgcatttatattactattatattaaattctgtttcaaaaatcttctgcacatgtcttaaatacaaatgtattaaatacaagctgctgctaagatgaaagttgctggccccatcacaatgggtatcttccaatgtgaataaattgccttggggaataaaatcagatttggaaaaaccttgaggatggttgccatcataaactcttagagtgtgacctgggtgtttttctttttctctgtaggatgttaatagtatcttgtgtcatgctaggatgtctaggacagagggcaatacaatgaggggaaggcattctgcgatgtccccagccgtctggcttgaagagtaacttgctgaagtgaggactctgtggaggagcaagttatacagaaagaagtttagttgtgatctgttgagttggaggtgtctacagggcatccaagcagacataggttgaggaggcagcatatatgtgaatctggagcctagaagagaggtaagggctggaaatagggatctaagacccctggacagttgtgagtgtgcacaatgagggtcagatgcagagaaaattaggagactacagagagcagaacccagggtggggatctgggagtcagcagttgggcatgggcctggtagcaaggcaagccaaggaggaggagagggggcagtctcagacaccaaggaggggagagtgactagaaagaaaaccttcttgcagagacataggggatggggaagaactgcagactgaactggggcaaaggactgttggccttaaccagagagatttgagggagagatgagctgagagccaggggatcctgccatgtcccagcataaaaacagtacctgacacagatgggtgcttgggagctgttgtcggatgaatgagtggacagatgcatggatggacggatggatggaaggatgatagattgatggacaaacagatgaacagatgaatagctggatggacaactggatggatgggtagacagaatgatctcagagatcagaaaaagcttcatgcactaagtgggactgaaccgcgtctccatgggtagaaagcagaggaatctccacttgagtcaggaatgacccagtgctctcaatccagggagaaagccagcctggcttcactggggacacttgtgtgggggactcagaggccctttaaatgaggccagacgaggttggacaggtccaagccaactcagcactcctctgccacactgcacaggaggggatgtgtcactcagggagttgtggccctatgggtcccagtgttgtcatcagcaccgacagcctcagagaggaaagacacacactggggtaactccaaggctgtgtgtgcacttgccttggacagcagacaggcacagggacacctctagggggctggccacccccctgcctcatgtctaggtcccagccccgcccactgcaaccctgtgcccgtcatgcccagcaggctcctgctccagcccagcccccagagagcagaccccaggtgctggccccgggggttttggtctgagcctcagtcactgtgttatgtcttcggaactgggaccaaggtcaccgtcctaggtaagtggctctcaacctttcccagcctgtctcaccctctgctgtccctggaaaatctgttttctctctctggggcttcctcccctctgtcctcccagccttaagcactgacccttacctttctccatggggcctggaggaggtgcattagtctccgggtaaccggcaggaagggcctccacagtgggagcagccggatgcagcctggtcccggggcctgagctgggattgggcagggtcagggctcctcctctcttccagggcagatgtctgagtgagggacagaggctggttctgatgaggggccctgcagtgtccttagggacattgcccagtgactcctggggtcaaggacagaggctgctggggtgggcctgggagctgctgagtctcatagtctaggggagcagccccaagaacagctgagggtctaggctgaggactggatgccaatccagcctgggaggccacacggcctggtgacacagaggtcaccaaggggagaccaatggagggcacagagagggctctgggtctaggctgcagctctgtggcctgtgctgggtcatgaggacatggggacacagagggacgggtgagactgggtgaggtgccagaatccaaccctcccaggacagtcaccagaaaggagacagtctcttagggcagagatgtgtctgtccctggagccccgtcacctctgggcccagtgtctctctgttcacggatcgcctcctgccttcctcaaagggcatgttagactcaggaaatgaccagaggggagtgaatgaggggtgcagagaactccatggctaccaggtgaagtttggggtcatcacaggctgctggggtgggcctgggggctgctgagtctcatagtctgtgggagcagccccaggaacagctgaggtgaagggttctgtggtcgggcttgtggagacaggaaacatctcagagcctcagaggaccctgaggcttgtctaggtggagcccactccttgccaggagagccaagtgggctgggctgggcagagcccggtgcctgtgagggataggaagctccagttcaaagcaggcttgggtctccccacacactgcctgccaggacagtcctacaggatgagcaggggacccacagttcacggaggaggctctaggtcctggagaataagtgggtgatggaggggctatagggatggaaataggatccaggtgtcaaggccagattctaaactcagactccagagatcagagaagaaggaacacagcctgccctgggtatatggagaaattgaggctgtagaggagaggggctgggccagacacctgtgaaaggtaacttgggagggctcctaggaaggcacagagctgtctgctctccacagggcatgagtggaaaggatgggaaagaagaggagagaaccccgggtggaccggatggccacactgtgaaccctcccagagactttagacagagagaggggctccacaacaccccggtattctgtctgccctctctcacccccttccctgtccacacaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcataggttcccaactctaaccccacccacgggagcctggagctgcaggatcccaggggaggggtctctctccccatcccaagtcatccagcccttctccctgcactcatgaaaccccaataaatatcctcattgacaaccagaaatcttgttttatctcattttttttctcacataaattgctagcctccccggggttctcagtgtggggtacagggaattctgcacccagtgtgaaaatcacccaagggaggaggctcacagcctccctgagtcatctccccagagggtccttcctctcccagtcaccccttctgcaactctccactgtacccctgagctaccagtctggcatcagttcagaccagtcccacaccctcctaaattttacttctcaataaatacctgatcatgtaaaacccagcatttctaatgtgcagtctctgtctggtcatgtgtctgggctgaagggtcactgctcagggacagggggcagttccaggtgagatcccatgtctccgtcatcccacaccccacccaacctgccagggaaccgggtgagctccctgtgccagtgggaactgcaatccaaggcacaaaattgtcctgcagtccttgcccacctgggaagggacaggggcccagtgagaggtttgctggcgccctgtggggagattcaggagaaatgaagggggtccccggagaccagatgagggctagaggcagaaataatggaaaaaggacacccttgactcaaggccacggtctcagcaggaacagaaggtgaaattccccattgcatacgaggaaccagtcaggagagtgtttactgggtgagggataaataactgtgctgccactgggaacttgtaaaaacattgggaaaggaaacatgcaagtgtctttctaagacttgtacaatggacattggctaagtaaacatactgacaagtcctgcactagggaaccagtttaatatgatgagccacagcatatccaaaagcatgttgatctccttcttcacctttagaagacccaaaacactctgaaagataccagcgtttcctggaactagtttgtggaatatggggtgaggttgatgcacatgatgttacgggtatatgatcacatggctgtgggttggggatcaggctcaaagttaacactagcgtggggctggatgtcaagcatgaagggtgtggaccactaagtcaggcccaggtagagttaatttctgattggtttgtggctggagcttgatgatggtcagtctgcaggagcaggaggatgtggggaaattgggaaaatgagaaaagtcacaaatccaagctcaaactctgcatctattgattgcctgggggaggctaatcagagttgaattcaggatgagcttcagggctgggtcagactggctaagagctgagtgaatgtgggctgatggctccaggcaaggcctggcctccactaggagtcagatcccacaaaccctcctgcccgcagagcaccctctccctccgtagctcatggtggcgccatgccccaccccatcccatgtacacctgctgcctcatctcagagacactcattccagtgtctctgacagcagatgatgtcagcctcctgggtgtggagaccccagctgtcttggagagtcctcagtgcctgggtactctcggaccccctgtctctgcctccagcacatcagagacgtagcagctgcctccaccagagctgctgggtgatcccaacaggccagggacagagcctgcaaagacaggaatctctgcagtcacaatgaggcaaagaaagagccccttagagcttgatcacagccacccctgatccaaatcccagcctctcattagaaggaggcttgagggttctagttgccacagcacctgtctgagcccatttcatggaggggaaaactgagatgaccaagggccagatccatagtcctgctgggcacaaggccatccccagcagctgcctaatctttcactgtgttataagtttccattatggaaaactttgaacacatacataaggagacagagaaataataatgcccccaagttcccatcacccagccccccaaataagcaattcacagacattactgacccacccatagcagaataacccctccattacacaataccagacatcacatcttttcagctgtaaatatcccatttctatgctggaaagatatgggcttaaaagtaactgcaatattattaccaaacctaaatagaaattatcactaattccctaatatcaagaaataatcacgggctcctcaaatccctcacaaatgccagaagcgtattgacttagttaagtgttggtgctgtggttattttggggttttgggtggtttatttcagaattcaatatggcatcaaatggtgatgggcgcatgtgctgtcaggccagttgtcactggtgaatatttcctcaattgctctagtgctgcctggcaaggcagagctgcaggagttgagagctgtccggggaccttcccacggttggaatacagccacacctcccaaaacaagaacccagggctatcatctacttctttttttttccccctgcaaaatggttctagcatggagggacttaactggattcagactagacattgcaaaatagcttccaaggacagggagctgctaacacggaggtcacccatgtcagattctcactcttgtagtaatgttagctgcataggatggtcaatagctacatccctcagaagggaaggaaggcagaggatgaggcttcagttcacctccttctcatgagtgctgcagagctctgtgaattcagaggtctgcagctgggctctgttcacccaggagtgtgcttcatgctctaggaaggagccactttgcacacagatgatccagggcccagcatccttccagggtgaataattaatgtcttctctcacggtgaactctaggattcaagccatctaatgtttttgaagccactgtcattatatttaattgatgatgacaggtggccaccaatgatgaatattttccaagggggagtctccccaagtggctttagacttcctcacatggccccaggggattaatggctcctgattactcagaggataagaggttctgtcttatcatgttcttttcttatttgtcttatgtgtctttcctgccccaggcctggatcccccactgatctcccttcccttagtgagaggtggtatttggagaccacattctggaggctcccttatgtcccgcattttgaaaaagacaacggcagccaccaccccagctgtcccacccaacatgaggccagattcaggggtgcagggatgctcccaaggttaccctaacagatgtgactggcacttcatattgggaccagccaggcctcactgaccaggcctatccaactagaactactccagaaggtggggctgaaacccacccaaggttcccagaacactgcactctagggcaatcagcctctgcatgggaggagaggagcaccctctgcaccaccccatggtgttaccaaaagttgaaccatgggttggttcaactttgcagagaagagaccacctaacccatctgtggaaattcactccttagcgacactaatgccctctaataaattcaatcctgggcctgagtgatggttggtgcaaaaaacaaattcaagatcccagtgtcctccagaagcctggatttccagggatcctgctgtgggtcacaggacgtcaccggtccccttctctttgtgggttgagtgtgggggccatgtggactccctcatgagcagatgccaccaggaccactggccccagcttcctccttcacagctgcagtgggggctggggctaggggcatcccagggagggtttttgtatgagcctgtgtcacagtgtgtggtattcggcggagggaccaagctgaccgtcctaggtgagtctcttctcccctctccttccccactcttgggacaatttctgctgtttttgtttgtttctgtatcttgtctcaacttgtggtcagcccttctccctgcatcccaggcctgagcaaggacctctgccctccctgttcagacccttgcttgcctcagcaggtcattacaaccacttcacctctgaacgcaggggcaggggactagatagaatgacctactgagcctcgtctgtctgtctgtctgtctctctgtttgtctgtctgtctctctgtttgtctctctgtctgtctgacaggcgcaggctgggtctctaagccttgttctgttctggcctcctcagtctgggttcttgtcggaacagctttgcccttgggttacctgggttccatctcctggggaattgggaacaaggggtctgagggaggcacctcctgggagactttagaaggacccagtgccctcggggctgatgctcgggaatcacagagctgggacccagagccaggatccagacccagaatgaggtaggaggtggaggggctgccctgggcgtctgggggcgctgccagggactgagccctgagccagcctgagactcaggaaaccccgtcaggagggagaagggagaagcagactctggacaccagaaagccaggggaagggtcacaaaaggagtggatgtgacggaagggcgggctcctgggtctcttcagaacatatccctgtgcccagggggatcagaggggcagagtcacctgcgtgaaagccccactgctatgaccaggtagccgggacgtggggtggatgccagaaaagactccatggaataagagagagcccaggacagcaggcaggctctccgatccccccaggcccttgccccatacacgggctccagaacacacatttgctggaacagcctgagggaccaaaaggccccagtatcccacagagctgaggagccaggccagaaaagtaaccccagagttcgctgtgcaggagagacacagagctctctttatctgtcaggatggcaggaggggacagggtcagggccctgagggtcagatgtcggtgttgggggccaaggcgcagagatctcaggacaggtggtcaggtgtctaaggtaaaacagctccccgtgcagatcaggacatagtggaaaacaccctgacccctctgcctggcatagaccttcagacacagagcccctgaacaagggcaccccaacacctcatcatatactgaggtcaggggctccccaggtggacaccaggactctgaccccctgcccctcatccaccccgcaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataggttctcaaccctcacccccaccacgggagactagagctgcaggatcccaggggaggggtctctcctcccaccccaaggcatcaagcccttctccctgcactcaataaaccctcaataaatattctcattgtcaatcagaaatcttgttttatctcattttttcttttctcacatataattcctagcctttcgtgggttctcaatttgtggtggaaagaaccctgaacccagtgggaaagttgcctatgtgaaggggttctcagttccctgggcatctctgcaggtaaggccttcctcacccagacaccccttcctcagctcttccactgtacccctgagccaccagcctcgcctggctgggaccaggggggtgtcacactctcctagattctgcctttcaacagaaacctaaccacgcatcacacggcacttctcgcatgccttctgtgtctgctccagtctctgggctaaagagttgctggtccgggacaggggataggtccgctcttggtcagatgccaggtccctgccatggcatccctgaccctatgcaacaagccagtgactctggtgagctctctgtgtcaggagaatccatgatccagagtttcatattgtcctgcaagcatctggtgggctgtagctcttgccaaactgggaaataccatggcccagcatcaggatgcaggacagtccggagagggaaatcaggagaagtgaaggggtctctggggagcccagatgtgggctagaggcagaagtaagggtgaagagcacctatgagtcaatgtcatggtctcagcaggaacacagttgaaaatccccattccacacgagaccgtttagcaggaaaggagtccatacttgtgctgccaccaggatgtcctgagaagccttggagaatgaaacatacaggtgcatttcctagacttgacaatgcacgttagccaagtaaaggcaatgaaaagttctctactagggaaataatttcctgtggtaaagcttagcttatgtaaagtcacatttatccatctggcacctctaaaagccccataatattctgcaagatactagtatgtcatggaagtagtttatgaaacataaagtgagatttaagaacaaagatgttacgggtgtatgataagatggctacaggctcagggtcaggctcgaggagtgaaggaggccgtgtcaaattcatgacaagagttggagctgggccaggctgggtcagggctgtgtgaatgcagacagagggctacaggcaaggtcaggcatccatgaacactcagctcccccagaccctcctgcccactgggaccttctccctcccttggtcacagtggtggagccttcctacccaaacctctatggaggccctggatgactgtgcgttcttagtgcccacgcaaacttagactccctgtctctgcctccagcacatcaggaatgtggcagctgagttcaccagagctgctgggtggtccgacagccagggacagagcccgcaaagacaggaagctctgcagtcacaatgaggcagagaaattggccccttggtgcttgatcacagccacccctgatccaaatcccagcctctgaattagaagaaggctaaaaggttctagtggccacagtccctgtctaagcccatttcacaaatgagaaaactaagaccacccaaggagggccagttccgtagcctgctgggtacaaaggccaaggtctacttcacacccagcagctgtccaaagactgacctgtgtcataagtttatattatgaagaactctgaacatataaataaggagacagaaaaataacagtgtcccatgttctcatcacccagcactcaaaataagcaattcacagatgatgccgacccacccacagcaaaataaattctcccttacacaacatttagaaagaaatacaagacatcagatctgttcagctgtaagtactccattactgtcctggaatgacatggaccttaaaataactataatatcactaccaaacctaaatagtaattatcactaattccctaatatcgagaaataagcagggtctcctcaaatgcatcagaaacaccagaagtgctttggcttagttacatgttggtgctgttggtatttgggggtttaagtttatatgaggagcaatatgacatcaaatggtgatgggtgcatgtgccatcaggctggttgtcactggtgaatatttcctcaattgctctagagcctcccggcaaggcaggagctgcaggagctgagagctgtctggagaacttcccctggctgctatacagccacgcctcctggagcaggaacctagggcttccctcagcttttattttcctggaaaatgattctagcatgaaggggattaacttgattcagattggacattgcaaaatagcttgcaaggacagggagctgctaccagcagagtcacccatgtcagattgccactcttgtagtaatgttagctgcataggatggtcaatagctacatccctcagaagggaaggaaggcagagggttgaggcttcagttcacctccttctcatgagtgctgcagagtgtctgtgatgtcagaggtctgcagctgggctctgttcacccaggagtgtgcttcatgctctaggaaggagccactttgcacacagaagatccgggcccagccatccttccagggtgaacaattcatgtcttctctcatggtgaactctagggttcaagccatctagtgctattgaagccactgtcgttgtgtttaattgatgatgacgggtggccaccagtgatgggtgttttccgagggggagtctccccaagtggctttagacttcctcacatggccagggataaatggctcctgattactcagagataagaggttctgtcttatcatgttcctttcttatttgtcttatgtgtctttcctgcccaggcctggatcccccactgatctcccttcccttagtgagaggtgatatttggagaccacattctggaggctccctcatgtcccccatttgaaaaagacaacggcagcctccaccctagctgtcccacccaacatgaggccagattcaggggtgcagggatgctcccaaggttaccctaacagatgtgactggcacttcatattgggaccagccaggcctcactgaccaggcctatccaactagaactactccagaaggtggggctgaaacccaccaaggttcccagaacactgcactctagggcaatcagcctctgcatgggaggagaggagcaccctctgcaccaccccatggtgttaccaaaagttgaaccatgggttggttcaactttgcagagaagagaccacctatcccatctgtggaaattcactccttagcgacactaatgccctctaataaattcactccttagcgacactaatgccctctaataaattcaatcctgggcctgagtgatggttggtgcaaaaaacaaattcaagatcccagtgtcctccagaagcctggatttccagggatcctgctgtgggtcacaggatgtcaccggtcccctctctctgtgggttaagtgtgggggccatgtggactccctcatgagcagatgccaccaggaccactgggcccagcttcctccttcacagctgcagtgggggctggggctaggggcatcccagggagggtttttgtatgagcctgtgtcacagtgtgtggtattcggcggagggaccaagctgaccgtcctaggtgagtctcttctcccctctccttccccgctcttgggacaatttctgctgtttttgtttgtttctgtatcttgtctcaacttgtggtcagcccttctccctgcatcccaggcctgagcaaggacctctgccctccctgttcagacccttgcttggctcagcaggtcatcacaaccacttcacctctgaccgcagggcaggggactagatagaatgacctactgagcctcgtctgtctgtctgtctgtctctctgtttgtctgtctgtctctctgttggtctctctgtctgtctgacaggcgcaggctgggtctctaagccttgttctgttctggcctcctcagtctgggttcttgtcggaacagctttgcccttgggttacctgggttccatctcctggggaattgggaacaaggggtctgagggaggcacctcctgggagactttagaaggacccagtgccctcggggctgaatgctcgggaatcacagagctgggacccagagccaggatccagacccagaatgaggtaggaggtggaggggctgccctggggctctgggggctgccagggactgagccctgaccagcctgagactcaggaaaccccgtcaggagggaccagggagaagcagactctggacaccagaaagccagggcaagggtcaccaaaggagtggatgtgacggaagggcgggctcctgggtctcttcagaacatatccctgtgcccagggggatcagaggggcagagtcacctgcgtgaaagccccactgctatgaccaggtagccgggacgtggggtggatgccagaaaagactccatggaataagagagagcccaggacagcaggcaggctctccgatccccccaggcccttgccccatacacgggctccagaacacacatttggctggaacagcctgagggaccaaaaggccccagtatcccacagagctgaggagccaggccagaaaagtaaccccagagttcgctgtgcaggagagacacagagctctctttatctgtcaggatggcaggaggggacagggtcagggcgctgagggtcagatgtcggtgttgcgcaaggccccgagagatctcaggacaggtggtcaggtgtctaaggtaaaacagctccccgtgcagatcaggacatagtggaaaacaccctgacccctctgcctggcatagaccttcagacacagagccccctgaacaagggcaccccaacacctcatcatatactgaggtcaggggctccccaggtggacaccaggactctgaccccctgcccctcatccaccccgcaggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttcataggttctcatccctcaccccccaccacgggagactagagctgcaggatcccaggggaggggtctctcctcccaccccaaggcatcaagcccttctccctgcactcaataaaccctcaataaatattctcattgtcaatcagaaatcttgttttatctcattttttcttttctcacatataattcctagccttccctgggttctcaatttacggtggagggaattctgcacccagtgggaaagtcacccaagggaggaggcttacagcctccccgagtcatctctctggaaggtccttcctcttccagtcaccccttccccaactctccaccatacccctgagcctccagcctggcctcagctcagaccagtcccacaccctcctcaattttacttctcaataaagacctgatcatgtaaaacccagtttccaatgtgtcgtctgtgtctggtcatgtgcctgtgtgctgaagggtcactgctctgggacaggaggcagtttcaggtgagatcccatgtccccgtcatcccacaccccacccaacctgccaggaaaccgggtgggctccctgtgccagggggaaccatgttccagagcagaaagttgtccctgaagagtggtccctgaaatgcagttcttgcccacctgggaaggatgtggagcctagtgaggacagagtggtggccctgagcagcggatcggggagaaacgaggagtgttccaggaccccctgctttgggctagagacagaaaacccttgagcccaggccaagatcagagcagaaacagggttgaacttccctgtcccatccatgatacccagttaggagaccatttactagtgccatcaccttacgtacattacaacattacgtgattgtgccatcaccccggagacatgaaaaaggctggaaaatggaacccttcagtgtagtttacactttcacaatgtacgttagctatgaaagatgctgacaagtcctgcagttggcaaaacagttcatgttacataaccttgcaagtcaagaattctattcagtgtcccaacccacttagccctagagcgctcttcaagacactggtggtcatttcactagtgctgggacatgggctgaggctgaggcacacagatgattcgttgtgatcaaatgggtcaggctcagggttaacactggccaggtcagaaagagagcatagggctgagatctcaaccatgaagagtctcgaattctaaagtcaggggacgcagtagagttagattatggttatggctggagccatgatggccagcctgtgtgagggtaggactcaggtggactgggtcaaatgagaaaggcaccatcccaagcatagaatcggcatccattggttgtctgatggaggctgtgtcaaaatcatactcgcccaagaatcagggccaggtcacactaggtcagggcagggtaagtgtgacttaagggctacaggcaggtcaagttttcatgggactcagctaccttagacccctccccaccagggcctactccctccctcaatcatgtggttcagcccctccatgtgcacctacaccctgatgtcagagacacaatcatcccagggtccctgacagcgagtgaggtggccttgggagatgcacttcccagccctcctcatcagtcttgggcactgtcagccccttctttggcctccagcacatcagcggtgtggcaggtgccttcaccagagctgctgggtggccaggccaggcctgagacagagcctgcaagggcagagaactctagggccatagtggggcagagaaggggttcctcttggagcctaatcatagaacccctgcctcaagtcacaacctacaagttagaaggaaacttaagggtcctgaattcccaccaccctgtctggccccacttcatagatgtgaacgctgagacccctatagcaaagaggaccgctttgatctccaccttctcaatggccctgctgggtaggatcccctctggatgtcccctggtgctgtcccaagactaatctctctaattactgccttgtaagatattacggaaactgacagcaagaaaataaaaaaacaggataatacagctcatgttgacccacccacaatcaagtaacctcttttacacagttgtttgaagcaaattgtagacatcatgtccattagtctaaatattccatttgtgtctctaaaaatatggacccccccaaaaaaactacattcttacaaacctaaatataaatatctaattctttcatatcaaaaaaagaatgtttcccatcaaatacttcacaaatatcctatgcttctttcactagacctgtgtttgtgttgttgttattctgtggttttcatttcatttctatgaggattcaatatggtttgaaattgtgactggtgactgtgttttagacctgttctgtctgcaggtatcttcctcattgatttttaatttccttgcaaggcaggagctacaggagctgggggttggtcccaggaccttcccatggtcaggatacagcctgtggcctccccaagctggaaacaagcgctcctctctgcttctgcgtttcctgaaaattggttcttggccagaaaggtttaacaaggctcagtgtgacttttcagcaagaccgcttggctactgggctcccatgtggggtcatctatttgtgacgttagctgggcttcacactttgtatccagtgccattagatggtatatggatgcaaggtgactgcatttcagttcgaccaccttttccttctactgactgtctgtaaaaggtgtgccctcatatgttctttgctcctctgggagtgtgattcttatttcagtaagaaatagcatagacatgttgagtctttcctttcatttagcatcttaataatgatgaccatgttgcctgccatctcgtgaagatgaacaattatttcatggtgagctcaaagttatgttactgtatgtgactcacttgagtccaccatggttctattttattgatgatgacaatgacccaccgtggcccactcagtgcctcttctggtggccccaggatcctcctgaaggaacccaggagacctcgatggctttccgctctctgttcacaatctatcctgggcacatctttctcctgccttgtgcctggaattgcccattaaccccaagtggactagtcccataactgggaggtgggatttagtgaccacacttgggtgcttctcacacagcccttttgagtcagacactccagacatacccagaaatgagacaagacctgaaagggtaacaggggcttgcttccaacttctccctggaggttgaggctggcatttcatactaaaacctagtgagacccatcccaaactaagacaacacaaggaggacggaagtgagacgccctggagttgtggttgtggtcacgttggagcttcccatgactgctgactctgcgaagctgcccctcctctaagcactcactgggacacctgaggacgcctcctgctcttaccctgtagtcacaccaagagatcagggttacaacaaccctatagagaatccctgtccccttccatgtcacttcactccttcgtgaagcaaatgccctcaaggagctcattcccattcctgggtcacagtcacctggaaaacctgatccagacaccaacctcctcagcctcgccatttccagacgtcccgtgttactgcatacgcttggtcgactgtcccatctcagcttgagaagggcaggcaggtgtgtggactctgctgagcaaatgccttccaggggcagtggtctggcttcctgcaccatagcttcaggtgggggatggggagggggagttaggggccccagggaagagtttttgtatgaacctgtgtcaccgcattttgtatttggtggaggaacccagctgatcattttagatgagtctcttcttccctttctttccctgccaagttggtgacaattttattctgatttcgatctttgtctgtgacttgccacagcctgtggtcagggtttcctttgggacctcggtcctgggaggctgatctctctcctccctattcagacccctgtatgcctcagctggtcactgagacaccttcatctcctctgaccccagaggcagggagctccaagacaaggccacactggtgtgtctcatgagtgacttctacccgagagccatgacagtggcctggaagatagatggcatcaccatcacccagggtgtggagaccaccacaccctccaaacagagcaacaagtatgcggccagcagctacctaagactggcacccgacagtggaagtcccacaacctctacagctgccaggtcacgcatgaaaggaacactgtggagaagacagtggcccctgcagaatgttcttaggtccccgaccctcacctacccacgggggcctagagctgcaggatcagggcatctgtctcccctcccactccaagtcatccagcccttctccctgcacccagtaaccctcaataaatatcctcattgtcaaccagaaatcctgctgtctgtcttcatttcttatctcatatttagtttgcaacctccttaaattctaagcaaggatgaggaaaatccaggtgcccagtttatcgggtgagaagtccatggtggtgccatcaccaggaacttgtggaaaggtctgggaatggaaactcacaggtgaatttcacagattttcacaatacagggtggctaagtaaagacacttacaagtcctgcaatagggaaacaggaagtccagaatcctgctcaccatcccagccaacttagtgagccctaggatgctctgcaagatactggtgttcacgtcgctagctctggaaagtggggtgaggctggggcacacgggtgatcagttatgatcagatgggcttagggtgaggttcaaagttaaccagcacgtggctgagatctcaaccatgaagttcccaattctaaagtcaggctctggggtggagtgagtatgtgcttggtgtgtggctgagcctgtgatggtcagctcgtgtgaggggaggactcctgtggactgagacaaatgagcaaagacaccatcccaggcacagaacgggcatcccacggttgtcggggagagtctgtgtcagagtctcattctggactagagtcaggctgggtcacgcaaggtcagcacagggtgaacatgacctaggggctatctataggcaaagtcaggctttcacgggatctcaactgccccaaacacccccatcccaccaggccccactccctctgtcactcacgttgttccgtcccctcaccccctgcaccatggtgcaccggcagcctcactcagagacaccctcatcccggggtccctgacagtgggcaatttggtcccttgaaggccaggttgacaggctcggttaatccatagtgcccgggctgggacccccactgtttctggttcatcagggacatggcagcagctgctgggtggccagccaggacaggaacagagctgcaaggcctgggggctttttccacaatgatacacaaagagaggggcccctttggagctcagtcccagccacccctgccccaaatcacagccgtgagctgaattgaatttcaggtgcccagagtccctcagcctctgtttgacccatttcacagctatgaaaattcaagcccatgggagacactgtcccaagcttcaccctctctataagttgtacatttttatgatgaagatctctgaacacaaaaatagggagacagaagaatagtaatgactccaaggttcccatcagccagtccgcagcatcatccattttcagataatgcggacccacccacagcagaataactaaactactccttcaagcaagggtgtgaagcaaatgctagtcatcacacacacaactggagagaatatcaaggatttcttgacatcaaaaatagttaatgagagtcttatcaaatgtcttgtgaatatcattgtgtctatttttgtcgactttgtggtgctgttgcatatttgtgatttaatttcatttctatgtggattaaatacttgacgttatcattggtgaatgtgtttttagaccccattccatctgcaggtgtctcccaaattgctctagctttccctggcaaggcaggagctgcaggagcagagagctggtcccgggacctcccacagtcgggatgcaggcgccacctccctgagcaggaacccagtgcttccctcaacctctcttttcctgaaaaatggttctagcatcaagaggctcaagggggttcaggctggacattggcaaactcgcttcccaggacacgtggctacttccagcaaagccacccatgttgtgttgtcattctttcagtgacattagctgcatttgatgatcaataacttcgcgcctcagatgagaaggaaggcagatggtcaagacttcggtccacctccttctcatgagggcttccagaagggagggcacagcagctgcaccgtgcgctcaggagtgtgcttcatgctttgggaagaagaaaaaatgtacattcttcccttttgttcaccactttgataactgatgatctggtgcccagccatcctccagggcgcacagcacaatgtagtaccggagtgagctctagcgtgtgaggacatctgacatgcgggctccactgcagatatactgaattgcaacgacaatgcggctacaaaacataaacatttaccactgggcgcctcctcaggtggcatctgattttctcccattgccccaggagcttccatggctcctgatttctcggaggatgagaggttctgtctcatcatgtccctttcctgccccaggcctgggatcccgcactgacctcacctcccttagcagaaggtgatatttggagaccacactcgggagctcctttatgtccctcacatttgaataaggcagtggcagccactaccccacctcacccaccaaaatgagaccaggttgaggggtgcaggagatccctccattttaccctggaggatggggctggcatttccagtggggaccagccaggcctcactggccaggcccatcccaactaggacaagcccagggaaggctgggctgaggctcctggagtcacagataggttcatgggaagcttcccaagacaccgcactctagggtaacccaaggccttcttcctggagggagagggcactctctgcatcaccccagggcgtcaccaagcagtcagtgtcgagtcagctccaccagggagaccatttatccctgaccatgggagttcactcctagtgacacagtgccctccaataaactcatccccatggctgcatgatggttggtgggaaaaccaaatccactgtcctccaggaaccaggatttctagggatccggctggtcacaggatgtcacctgtccccttctctctgtgggggtgagtgtggcagccgtgtgaactccctcatgagcagatgccaccaggggctgtggcctcagcttcctccatcacagctgcagcgggggttgggggtagaggcgtccagagagggtttttgtatgagcctgtgtcacagcactgggtgtttggtgaggggaccgagctgaccgtcctagatgagtcttttccccctccttccctggtctccccaaggtactgggaaattttctgctgcttttgttcttttctgtatcttgtgttgacctgtggtgatgctttctctctggagcctaggccctggtcaaggacctctcccctccctgtttagacccttacctcagtgggtcaccaagaccccttcacctctgacctcagatgtagggcactagactggatgacctactgagactcatctgtctgtctgtctgccagagccaggctgcttccctaaaacttgctcagttctgtcctccccccacctgagcttctgtctaaacaaactttgtgcaagagaaactgaggccccatctcatgaagggagagggaacaaggggctcgaaggagtgaacacctggtggactttagaaggacctgaaaccctcagagccaagataggggaatgaaaactcagagtctctacggagaccatcccctggactgtgggactctggatccaggctgggaacaaggtaggaggtgcaggggcctctccaggtttctgtgggctcccagggagagagccctgagctggcctgggacccatgaagccctgtcaggagggacgggaaggctctggacatgaaggagccaggtgaagtgtcacgaaaggccatggcattcagggaggtggctgatgggtctctgtgggaggcacccctagaagcaggaacccctgagttcaccgacagcatatcccaaggcagaaaaactgtagattggccctaaacacagagagactctaacacagactccacagacaaagagcccaggacagacagacagtgggacttgggtgagcaaaggccctgactccactgcagaagatccaggagagacggatgtgggtacaaacaagagctcttacgtgagagacccactctcccccaacccagagcagctgtgtcaggtgagaaaagtttccagagtgaatctaacaagaggctcacagagctcagagaacacagccagagccggatcacgtcaggatgacagggtgaggcagcgtcagggcaattgaaggtcactgtctatacaaagcccaagtcttggaagatttccaacaggcgatgggcaggggcaagctcagaggttttctgactgacagagttctttaggacatgatgtcacaccttctgagacagtgtggaccacatctgagaggtcctgcactggtactggggggcctgaacactcctcatgcactgaggtcaggggctccccaggtggacaccaggactctgacctcctgcccctcatccattctgcaggtccgctcaaggccacccccttggtcactctgttcccgccctcctctgaggagctccaagccaacaaggccatgctggtgtgtctcataaatgacttctacccaggagccatagaaggaaaatggcaccctagtcaccaagggcatagagacaaccacaccctccacacagagcaacaacaactatgcggccagcagctacctgagcctgacgcccgagcagtggaagtcccacagaagctacagctgccaggtcacgcacaaagaaagtaccatggagaagacaatggcccatgcagaatgttcttaggcccccgaccctcaccccacccacaggggcctggagctgcaggatcccagggcagaggtgttccctcccaccccaagtcatccagtccttctgccgacacccaataaaccctcaataaatgtcctcctcgtcaatcagaaaacatgctgtcttgctcatttttgtttatccactttccatcctaaatttttttatctctaaagatcaacagaatcctttggacttgtgacaatgcagcctgggaccatcttcgtgtttctctgagccatacttttttccatcaccttatccccatagaatgttccagaacaaggaaatagtcttcagaaggaaccagacatcttcattcttacccacgtctctgttgtgcttcctgctgccctgtgagcacgtccacctctggctgcccggggacacaccctccttacatccctatggccccgggagaacatctcagctcccttctttgttgtcctcggccctcactgtccagatgccatccctctctcaaggtgtttgtccatccccctccaaggccatgtggtcaccccacctgtatttcccctcatcaaagcctagagcatctccccttctccatgggcacactctgttcccgtcatcccagggaccctgccctcagcccccacaggccctccctataataatcactgatgggatgcttaccccctggaccctggtcactcaccccacatccccctctggtgcctctcatcccacatgtgtcaccggatgccccagctgtattcaacattcctaggggacaagggtcggctctgtcctcactgacaccattatgaatgcctaatgtcctgctgtgacaagttgtcaattgactggtgagttcaaagtcagattctggggttatgccaggagcttgggtgacgctgattcttcttctgaagaactcagcatgggatctgagctataatacacacacacgtgtacacacacacaatgcacataaacacacatgtccatgctgggcatccttctgggaggatcaggaaggtagaaggggctgcagtcaggcccaggtcaggacatgagcaaagcaaagctgagacctgctgtctccaggctttgggactagggggtcgcccctggatacgggagggtggggcagggttttctgtccaggccaagagggagacaggagaagatgtggacgggattctgatcagggagcagcaggccacatctcagcagtcaggggaggcgcttgctgtggggaatactgttggggactgcatggtgccaggagtgtgtcccaggtgtgagctcttcaggtgatgggcccagaccaggatgcaggtccaggctgtgtgtgtccaggccccacactgcacatgcccatccactgtggtgccccccgtgctggtgacactccagagcacaggctgtgtgggtcacccgtgcatgtaggggactgcagagcacacacatgtgggtgtgtgtgtaagccgggggagctgaagtggacagcagagatcccccacacgtagagctcctggaggaaaccacactgccagtgaggcctatgacctcccttgagaagctctccttctggcatgcagctctaatccaggcacacatggctataggtaggaggctgtcccatgggttctaggaggcggacttgggcagtgatctgtgagctgctccttcaggacagctgggccaaggatgctggagctcagaccttgcagtggggtctgtgtccctgtggtcaacgtcattcttcccacaacccccaattcccattccaagcccatcccatcataacctccagtgctgaggatgtggggggcatcaaggaccctccccaccattcccaacttctcaaagaggtcccagctgcaccatgtcagtggcgtcacccagccgctcacctccccttcctccctggcattcctgacaccagctgatccaggccacccaactcctcagaaatgcaattacctgggagacaattccacacacagacgtgtcaaccttccatgatgaggtggatgaaacccctaagcccccaagaagtgatattcaggtcagtagaagtgacccccttcaccccacctatggctcacccacccatcagaaaagggggctggaccctgggccttctgagcagctccagggggttggggggcggtggcctgggtcgggtgtatcaggagggtttgtgtgcagggttatatcacagtgtaatgtgtcggcatggaccaagtgaccgtcctcgctgagtccccttttctactctctgggtctagggtgagatctggggagacttttctgtcctttctgttctctctagggtagaggtctcaatctctctggggtctgccaccattgccttttttcctggccccaaattcctccagcctgtcccttcttaggcacctggtggggcatgatgggaagatcccagtgacctcttaatgctcctgctcaggcctgaacctgccgctgtcactcagggggcattctcccctgggctctgggatgttctgctccctcgcacagagtctccagtccccaaagaccagcagagcaggggctcaggctggggcaccagggccatgggacaggggaaggatgctggaaaaagttcagcttcccagggttctgggtcccaactatggggctgctttaaacaccaagaagggaggcctttgactggggacttggggaaatgaagggggacaaggatggaggaagaatgtcctgtgaggtggcgccagggtgctgggtccctcctcctcccccgggacagaggctgccatggacagggtggttctcaggacactcagtccaaggttagagcctccccatcccacccaaaaagagagacccccaaagcagatgctgagggaggcactcctggtgggcgcaggtgacagggacctgtcaggacagacatttgtcctaggacagccagatctcccaatgacagggagaccccatagagcagacacagcgccaggctcagaacagaaaatatacctcacatgcaagccctccatctcccggactcccaggacccggctcccaggactgacatcccctccccaccaaggggcctctgtgggaaagtggggcagagactgcaatgatggtgctgggggatatgtgaggaagaaattcacttgtcaaaagggaggagaacctaaaaacaggacacagatgccctcgtgaacagacccagaggagggacctggcaggggatgttcagacagagacgtcccccaaggaaagacgaggcccagggctgaacccagcagcgtcggggagcataggtgaggtgagagctgctgataaggaggggagaatctggagaggaggtggagcagaagaggcctgattctcctcagtcctcagatgctgaggagtttcccatcaggaggtaagcgtccccgccaccgccaagttgacctcagtacagcaagggcccagcctgaggtccctatcctgggccttagtccttcacccacctgaaaactgaggccaggggtccccaggtggacaccaggactctgaccccctgcccctcatccaccccgcaggtcagcccaaggctgccccctcggtcactctgttcccgccctcctctgaggagcttcaagccaacaaggccacactggtgtgcctgatcagtgacttctacccgggagctgtaaaagtggcctggaaggcagatggcagccccgtcaacgcgggagtggagaccaccacaccctccaaacagagcaacaacaagtacgcggccagcagctagctacctgagcctgacgcctgagcagtggaagtcccacagaagctacagttgccaggtcacgcatgaagggagcaccgtggagaagacagtggcccctgcaggaatgctcttaggcccccgaccctcaccccacccacaggggcctggagctgcaggttcccaggggagggggtctctctccccatcccaagtcatccagcccttcttcctacactcaataaaccctcaataaatatcctcagtcaaccagaaaccctgggttttgtgttttgcttctgtttatacattttctaccctaaattggtccaacctgcaagatgaacgaaattcttttagccttgtgggaatgaagtctggattgtttttgaggggtagttccaccatcacctagaaaggcccagagaatattccagaacagagccttgggcagcaggccccaggactgcccctttcccatacgtgctgtgtttgtgtggacatgtccacctccagcggcctgggagaccgctccttggttccccatggcccagaagaacgagtaccctccacggatcatcaggcctcacccctggacagcctccctctctcaaggggtccctccttctgaggcggaaggagcctctccagtccatgggcccttggtctcccctactccttaggggcccaccctgttcctgactcccaggaccctgccctctacccccacatctatctctgaagcaggcacagactggaccctgaccctctggacccaggtcactcaccccacaggccagctgggtacccctggtcccacatgtgaccagtgtcccagttatactcaagtcccctggggataagggtcaactctactctctctcacccctaagtaaccagcccaaaactgaccacacctcaagtacctaatgtccagttatccacggatggtcagtggggctggggaggtcaaagtcaatcatgagattccagagtggtgccatagacgtgcctgtaaaccaattggtctttttaagagctgtatgtgggatctaagaaccgggttgatgattgtcccaggaggtagcacaggatggggggcctgggctatgagacaaacacacatacatacatgttcacaaacataaacacacatgaacacctcataggcacacaggtattcacaaacaaacatgcctgcatgcaaatacatacacacacactctcctgtgctatttagaatcattaccacatgggcatcccactaggggctgtgctgggcatccaggaccctcccagggaactcggatctccataggggaccagctccaccttccctgtggcataaacaggcttctcacttcttccatggtgacagtctccaaaggagctgagccgggacacatggcttcctccaggacagagagtagcaggggacccagatgtgatgtgcagacatgcctcctccctctcccctctccctctgggggtgaagggatccctgacttccagtaaagtgtgcatccatgcatggaggggccatccccctcaccctcctatcggccagacctgcttcatgacagggccactggacctgggcgcccaagctgctgcagcggggaacgggtcgggtgtgtcaggaggggtttgtgtgtggggctgtgtcactgtgtgctgtgttcggaggaggcacccagctgaccgtcctcggtaagtctccccgcttctctcctctttgagatcccaagttaaacacggggagtttttccctttcctgtctgtcgaaggctaaggtctaagcctgtctggatgtctggaatctttgcccctccttgcctgggctcctgctcttctgtgattctgtcctctgtgggtcccagttacggggctgcattaaacacagtgacaggaggcctttgactgaggacttggagagaggggggaggaaatggcaggaggacaaagatagaggaagaatattccgtgagaagggggcccacagcgctggtcacacgccatccccaagacaggcaggacaccacagacaggtgtgggtctcagaaaacacaggcccaggatgagggagcatattgatattaccgctggagaagacctcagagcagatgcccaggacagggacttctggtagggacggtgactgggacgggtgctgtttgtcagggaaaacccactggagagtcagatcccccagataacttctcacgacatggagactctttcgaacagacaaagtattgtgttcagctcagggagtaaaaaaaaaatgcctcaaatggaggcctttgatctactggaatccagcccccaggctgcaccctgtctcaccaggcagccaagaggggtctctgcagggaggtggggtgggggctgcaatgatggcaccagggagatgtgtgggtaagaaacccactccctgtgagagcgaagagcctgaacccaggaccaacagctgccctgcatgaagagatgagaacaaggggaactggtaggaggtgttcagacagacacccccaagatagacaaatacccagggtgagatgtggtcctggactccatcccatccagtgtggagccagcaccggtgggggtctataggtgatggaaaatatgaaaaagagacagatccaagagggtctgtgacccccaagagtgggggcaactcccatctgacagcaagtgtctccactcaccgctgacctgacctccagtccagcaagggtccggcctgaggtccctgccctgggccttagtcccatacccacttcaagactgaggtcaggggctccccaggtggacaccaggactctgaccccctgcccctcatccaccccgcaggtcagcccaaggctgccccctcggtcactctgttcccaccctcctctgaggagcttcaagccaacaaggccacactggtgtgtctcgtaagtgacttctacccgggagccgtgacagtggcctggaaggcagatggcagccccgtcaaggtgggagtggagaccaccaaaccctccaaacaaagcaacaacaagtatgcggccagcagctacctgagcctgacgcccgagcagtggaagtcccacagaagctacagctgccgggtcacgcatgaagggagcaccgtggagaagacagtggcccctgcagaatgctcttaggcccccgaccctcaccccacccacaggggcctggagctgcaggttcccaggggaggggtctctgcccccatcccaagtcatccagcccttctcaataaatatcctcatcgtcaacgagaaatcctgctccctctcttcttttcttatctcacacataattggaggcctcccctgggttctcagtgttgggtgggggaatcctggcacccagtgagaaagtggccctgagggagaggcacatagcctcccggggtgtctcctggtgaaagaggcccggataggagaagtctgatcactgaacaccagtccctctgccctttcattcctcccccttctcctcaaagcatagccccccacctccctgcctcctgcctggatggagttgtctctggctgggactccagttacaccctctatctctgccctaacagagacacccttcacccaccatctcttytcccagcctgaaaaccctgctccaagcctggagcctctttgcccctggcccttgccccggaatgccctcctccctctgtgcccagctcagctcmcacccaccctcaccccctccctgtcatccctgagagccagactgtccaggacactccagsaccactgacttctcaagctgttcaatgagggacccacctcgattcctcacctgacagcaggttcagacacttaagaggaatgggaggaagccaggaaggagactcacacgaaaggcctacaggaggctcgggacacttggaaaagataggctctgagctccctaggaaccagtgcaaggagggtaataagcacttcaccacttctgtaaatttaaacaaaccctttgctgaagggaaggtggtgttctcaatgctggggccagtcatctcgtcacaactaaaagtgggaacatgttctatccaaggcctcagtagtaatgggatgtgaaccttgacacacgcacaggtaactatctggcagcccttagacatggtgattctctcaagtgcgtgaacagaaagtcactggccgtctttggggacatcatttttgggaacacttctccattggatcccgggccttcactggaaaaccaggtccctcatcaggactttctctgtgaggttccctgagtctggggctccaactgctctctgagaggaaggggtggggaagagaacacgacccagccccacccagcccctcctgcatggcccctcctctgtagagtgggggcacctgcaggggtcaggcctgggttactgtgggtggggcctgggaggggcccttgtggcatcctggggagtctctcccacggctcctgtttgactttcctgtggctgctggctgttatcacaaatgacccctaatttagtgccttaaaacatcacacactgactctctcccaggtctggaggtcagaagtccaaaatgagtccttgggggctaaaatcagggtcccagcagggccggctccttctggggactctggggagaattcgtttccaggacttttccagcttctagaggacaacctcagctcctggccccttcctccaacttcagagcctgagctcagcatcttcaaatctctctgttgctcttctctccctccctaccttcttctctccctctcttccttctctctctctccgtctccctccatccccacatcttgtgctctcactctgaccctcctgcctccctcttgcttctataaagaccccagcttggagccacctggataacccaggagcatttgccacctcaggctcctaattgacatgacatctgcaaagtccctgtggccacacatgacacatagacaggtccaggatggggacacgacggtccccgcggggcctaatttagccgaccacggcccctttcccgagttcctctcccttcctccatgccctgcccagtccaggctgagccacatctctggggggacgggccagcccagcccaagccctccctatccctggtctcttgtggttccttctcctcctggggtttcaggaaggtactgggagtttagctcactagcctctagggaactagagttccagcaagggacctgagactaaatgggatccagagcaaaatcacccttggaggaaggatcctattaaagatcctcgctctaggatggtgacatcacagcccaggagagcctgagtgacctgcagacaggttcactagtagagagcaccacaaacctgg

SEQ ID NO: 8 - DNA, 1120 bp, Homo sapiens
gaccagcctacagccgcctgcatctgtatccagcgccaggtcctgccagtcccagctgcgcgcgccccccagtcccgcacccgttcggcccaggctaagttagccctcaccatgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctagagtcagcttacatccctgagcaggaaagtttacccatgaagattggtgggattttttgtttgtttgttttgttttgtttgttgtttgttgtttgtttttttgccactaattttagtattcattctgcattgctagataaaagctgaagttactttatgtttgtcttttaatgcttcattcaatattgacatttgtagttgagcggggggtttggtttgctttggtttatattttttcagttgtttgtttttgcttgttatattaagcagaaatcctgcaatgaaaggtactatatttgctagactctagacaagatattgta

SEQ ID NO: 9 - DNA, 456 bp, Homo sapiens
ttttttttttttttttttgccatttatttaaaacttttattaacgcttgaagaaaaataatgcaatgtgacaatgtacaggtcctgttgcctaaatccgtagtagaaacagatattatcacttagcaagctcacgtggtgccaattctgagatcagacggggttgttcctccttaggaagtggccactggaagcattgtttttccatgctatttccgtgaagccttttgcttggttcgagtttaaatttctccctttgtgtgagtatgactatagttctggcctggtgttttctatttatttagttttagatgtcagcattttactatacttggtcctctcacttcagaataacagggctatttattgatacaaaggagaggtgttcagatcatcttgttaagatgcagagctcaaaataaacactaaatctttatttggagatccacatccttcc

SEQ ID NO: 10 - DNA, 2064 bp, Homo sapiens
agaaggcagacgcatcccgaactcgctggaggacaaggctcagctcttgccaggccaaattgagacatgtctgacacaagcgagagtggtgcaggtctaactcgcttccaggctgaagcttcagaaaaggacagtagctcgatgatgcagactctgttgacagtgacccagaatgtggaggtcccagagacaccgaaggcctcaaaggcactggaggtctcagaggatgtgaaggtctcaaaagcctctggggtctcaaaggccacagaggtctcaaagacctcagaggctcgggaggcacctgccacccaggcctcgtctactactcagctgactgatacccaggttctggcagctgaaaacaagagtctagcagctgacaccaagaaacagaatgctgacccgcaggctgtgacaatgcctgccactgagaccaaaaaggtcagccatgtggctgatacgaaggtcaatacaaaggctcaggagactgaggctgcaccctctcaggccccagcagatgaacctgagcctgagagtgcagctgcccagtctcaggagaatcaggatactcggcccaaggtcaaagccaagaaagcccgaaaggtgaagcatctggatggggaagaggatggcagcagtgatcagagtcaggcttctggaaccacaggtggccgaagggtctcaaaggctctaatggcctcaatggcccgcagggcttcaaggggtcccatagccttttgggcccgcagggcatcaaggactcggttggctgcttgggcccggagagccttgctctccctgagatcacctaaagcccgtaggggcaaggctcgccgtagagctgccaagctccagtcatcccaagagcctgaagcaccaccacctcgggatgtggcccttttgcaagggagggcaaatgatttggtgaagtaccttttggctaaagaccagacgaagattcccatcaagcgctcggacatgctgaaggacatcatcaaagaatacactgatgtgtaccccgaaatcattgaacgagcaggctattctttggagaaggtatttgggattcaattgaaggaaattgataagaatgaccacttgtacattcttctcagcaccttagagcccactgatgcaggcatactgggaacgactaaggactcacccaagctgggtctgctcatggtgcttcttagcatcatcttcatgaatggaaatcggtccagtgaggctgtcatctgggaggtgctgcgcaagttggggctgcgccctgggatacatcattcactctttggggacgtgaagaagctcatcactgatgagtttgtgaagcagaagtacctggactatgccagagtccccaatagcaatccccctgaatatgagttcttctggggcctgcgctcttactatgagaccagcaagatgaaagtcctcaagtttgcctgcaaggtacaaaagaaggatcccaaggaatgggcagctcagtaccgagaggcgatggaagcggatttgaaggctgcagctgaggctgcagctgaagccaaggctagggccgagattagagctcgaatgggcattgggctcggctcggagaatgctgccgggccctgcaactgggacgaagctgatatcggaccctgggccaaagcccggatccaggcgggagcagaagctaaagccaaagcccaagagagtggcagtgccagcactggtgccagtaccagtaccaataacagtgccagtgccagtgccagcaccagtggtggcttcagtgctggtgccagcctgaccgccactctcacatttgggctcttcgctggccttggtggagctggtgccagcaccagtggcagctctggtgcctgtggtttctcctacaagtgagattttagatattgttaatcctgccagtctttctcttcaagccagggtgcatcctcagaaacctactcaacacagcactctaggcagccactatcaatcaattgaagttgacactctgcattaaatctatttgccatttcaaaaaaaaaaaaaaaaaaaaaaaaaaaaaaaaaaaaa

SEQ ID NO: 11 - DNA, 2631 bp, Homo sapiens
atgaccacctcagcaagttcccacttaaataaaggcatcaagcaggtgtacatgtccctgcctcagggtgagaaagtccaggccatgtatatctggatcgatggtactggagaaggactgcgctgcaagacccggaccctggacagtgagcccaagtgtgtggaagagttgcctgagtggaatttcgatggctctagtactttacagtctgagggttccaacagtgacatgtatctcgtgcctgctgccatgtttcgggaccccttccgtaaggaccctaacaagctggtgttatgtgaagttttcaagtacaatcgaaggcctgcagagaccaatttgaggcacacctgtaaacggataatggacatggtgagcaaccagcacccctggtttggcatggagcaggagtataccctcatggggacagatgggcacccctttggttggccttccaacggcttcccagggccccagggtccatattactgtggtgtgggagcagacagagcctatggcagggacatcgtggaggcccattaccgggcctgcttgtatgctggagtcaagattgcggggactaatgccgaggtcatgcctgcccagtgggaatttcagattggaccttgtgaaggaatcagcatgggagatcatctctgggtggcccgtttcatcttgcatcgtgtgtgtgaagactttggagtgatagcaacctttgatcctaagcccattcctgggaactggaatggtgcaggctgccataccaacttcagcaccaaggccatgcgggaggagaatggtctgaagtacatcgaggaggccattgagaaactaagcaagcggcaccagtaccacatccgtgcctatgatcccaagggaggcctggacaatgcccgacgtctaactggattccatgaaacctccaacatcaacgacttttctgctggtgtagccaatcgtagcgccagcatacgcattccccggactgttggccaggagaagaagggttactttgaagatcgtcgcccctctgccaactgcgaccccttttcggtgacagaagccctcatccgcacgtgtcttctcaatgaaaccggcgatgagcccttccagtacaaaaattaagtggactagacctccagctgttgagcccctcctagttcttcatcccactccaactcttccccctctcccagttgtcccgattgtaactcaaagggtggaatatcaaggtcgtttttttcattccatgtgcccagttaatcttgctttctttgtttggctgggatagaggggtcaagttattaatttcttcacacctaccctcctttttttccctatcactgaagctttttagtgcattagtggggaggagggtggggagacataaccactgcttccatttaatggggtgcacctgtccaataggcgtagctatccggacagagcacgtttgcagaagggggtctcttcttccaggtagctgaaaggggaagacctgacgtactctggttaggttaggacttgccctcgtggtggaaacttttcttaaaaagttataaccaacttttctattaaaagtgggaattaggagagaaggtaggggttgggaatcagagagaatggctttggtctcttgcttgtgggactagcctggcttgggactaaatgccctgctctgaacacgaagcttagtataaactgatggatatccctaccttgaaagaagaaaaggttcttactgcttggtccttgatttatcacacaaagcagaatagtatttttatatttaaatgtaaagacaaaaaactatatgtatggttttgtggattatgtgtgttttgctaaaggaaaaaaccatccaggtcacggggcaccaaatttgagacaaatagtcggattagaaataaagcatctcattttgagtagagagcaagggaagtggttcttagatggtgatctgggattaggccctcaagaccttttgggtttctgccctgcccaccctctggagaaggtgggcactggattagttaacagacaacacgttactagcagtcacttgatctccgtggctttggtttaaaagacacacttgtccacataggtttagagataagagttggctggtcaacttgagcatgttactgacagagggggtattggggttattttctggtaggaatagcatgtcactaaagcaggccttttgatattaaattttttaaaaagcaaaattatagaagtttagattttaatcaaatttgatgggtttctaggtaatttttacagaattgcttgtttgcttcaactgtctcctacctctgcctcttggaggagatgggacagggctggagtcaaaacacttgtaattttgtatcttgatgtctttgttaagactgctgaagaattattttttttcttttataataaggaataaaccccacctttattccttcatttcatctaccattttctggttcttgtgttggctgtggcaggccagctgtggttttcttttgccatgacaacttctaattgccatgtacagtatgttcaaagtcaaataactcctcattgtaaacaaactgtgtaactgcccaaagcagcacttataaatcagcctaacataag

SEQ ID NO: 12 - DNA, 116 bp, Homo sapiens
  misc_feature (77),(97): n is a, c, g or t
gatcacttggatatcattatgggaaaaataaaagaatttgacccctatcttacatcataaacaaaggtcaatcgtangtgggttataaagctaaatntttaaggttgccccttaaa

SEQ ID NO: 13 - DNA, 2629 bp, Homo sapiens
gtgagccaccatgcccagcccgggaccttttttcttaaagaagaaattatactgtatgtgtgtatgtgtgtagagagtgtggtgaggcttaaagacaatgggcttcaaagtctgccagaagggccccagtcaagttattcagcctcagtttgctcacctgtaattgggaataattatgtgtgatggtgttatgtaaacacctggcacagtgcctggccttctcctttacctgcttttcttcctgctccttatcattgagctttgaaagactatggtaaatgtttccctagtaatatgactaagtacttactggggtttgacgggggaaagaatatgactattttatccagtactgactttatgcagaagattttaagggtggggtcactgacattggtccaagggaggaaatgtacagtgttaagggggctgagcgagttttagccaaaactctgaaaaacgtgacagatgctaagcatttaatgccacgtaagctaaataatgtagggccagctgccgttgaagcacaactaagataagctaataattagttgtactaggatttatacctcatacagacagtagttgatgtatcagaatgcattctccgtgatggaggttttcttggttcctggctcataattcttagggaagtctgcgttctgctgatcacgatcactttattgtcctacccagtatcactgatatgtactttgttaaataaaaatacaaataacctgaagaggtattatttgaaagctgtgtaactccatggtatggctagtgtgataattgtcttctgataatgcatatgtagtgaattatggccgaggttagtttccagtactggttataagtaagttaaatttctgaaatgttttgggcactgttcagtaacctttggagtagcttgcagtgtctttactgctttcaagtgctgatgtggaggaatcattagtgttgggcagggaaacaggagctgtcagactgagagggccagggatgttcattgtggagattaaatgttctgagtaagtgagtttgtagacattttcttgtaagaaaatgttccatgctttactctgttttggacattttcttttgaaaaattttgagatagaaaccttaatatcatttcatatatttacagcatatagttaatttgagtagagtcaaattaatcttactttagaatttgattactaagtattagcatggataataatgcttcttttgcttaaatgtaaaaattaggccgttttactaggtgtactttgtcttaatcatattcctatttttttatacagctgtgcaaccatattgcttctgggaaaaaatgtcaatatgttggaaactgttcctttgctcatagtcctgaggaaagagaagtttggacttacatgaaggagaatgggatacaagatatggagcaattttacgaactatggctcaagagtcaaaaaaatgaaaaaagtgaagacatagccagtcagtcaaacaaggaaaatggaaaacaaattcacatgccaacagattatgctgaagttacagtggactttcactgctggatgtgtgggaaaaactgcaacagtgagaagcagtggcagggccacatctcctccgagaagcacaaagagaaggttttccacaccgaggacgaccagtactgctggcagcaccgcttcccaacaggctatttcagtatttgtgataggtatatgaatggcacctgcccagaaggaaacagctgtaaatttgcacatggaaatgccgaacttcatgaatgggaagaaagaagagatgccctaaagatgaagctcaacaaagcacgaaaagatcacttaattggcccaaatgataatgactttggaaaatatagttttttgtttaaagatttaaactaatatgctggcttttatgtatgatacctaatcagagcattgaccagaaaaattgaaagtgttctgaggcacatagcagaggagctgcagatttcctgcttgtattggcgtatatcgttcctcctgagcagcaacccacagtaggtaggaaaatgggctgtttcacaggcctggccacgctctcacggaaccactggcatcagatggtgaagtgactgctacccggttgccatctgttgaacagacttttggatgaagtgtgttggggaagaggataaggttatatctaggacaactctttgagttggtccttcatataagaattcgtgacggtaagagaataaacacttgtactgggatcagaatacatgatggatgaaattctttacatgttttagcagaatgaatttgtttaatataataaagtttgctacttatctgtatgtaggttgctaaaaaggattttcttaactcagattttaagccaaataaccatttaacactagtatttgttaaatggggtatttttctgtatttgtatgtttcactataataagggaattaaggataatgtgcattgagaatattttgaaaaataattgactcaaattttatttcttggtcttttgctgtttaaatgatgattttgaaagattaaacctgtactgttggtattgtgttagtgtatggaccaatactgcctgtaataaagattttatatatagatg

SEQ ID NO: 14 - DNA, 1466 bp, Homo sapiens
ggttctgccctgctgtcctctgccaccctaacagcccacttgacgaggagaatctgacccaggagaaccaagaccgagggacacacgtggacctcggattagcctccgccaacgtggacttcgctttcagcctgtacaagcagttagtcctgaaggcccctgataagaatgtcatcttctccccactgagcatctccaccgccttggccttcctgtctctgggggcccataataccaccctgacagagattctcaaaggcctcaagttcaacctcacggagacttctgaggcagaaattcaccagagcttccagcacctcctgcgcaccctcaatcagtccagcgatgagctgcagctgagtatgggaaatgccatgtttgtcaaagagcaactcagtctgctggacaggttcacggaggatgccaagaggctgtatggctccgaggcctttgccactgactttcaggactcagctgcagctaagaagctcatcaacgactacgtgaagaatggaactagggggaaaatcacagatctgatcaaggaccttgactcgcagacaatgatggtcctggtgaattacatcttctttaaagccaaatgggagatgccctttgacccccaagatactcatcagtcaaggttctacttgagcaagaaaaagtgggtaatggtgcccatgatgagtttgcatcacctgactataccttacttccgggacgaggagctgtcctgcaccgtggtggagctgaagtacacaggcaatgccagcgcactcttcatcctccctgatcaagacaagatggaggaagtggaagccatgctgctcccagagaccctgaagcggtggagagactctctggagttcagagagataggtgagctctacctgccaaagttttccatctcgagggactataacctgaacgacatacttctccagctgggcattgaggaagccttcaccagcaaggctgacctgtcagggatcacaggggccaggaacctagcagtctcccaggtggtccataaggctgtgcttgatgtatttgaggagggcacagaagcatctgctgccacagcagtcaaaatcaccctcctttctgcattagtggagacaaggaccattgtgcgtttcaacaggcccttcctgatgatcattgtccctacagacacccagaacatcttcttcatgagcaaagtcaccaatcccaagcaagcctagagcttgccatcaagcagtggggctctcagtaaggaacttggaatgcaagctggatgcctgggtctctgggcacagcctggcccctgtgcaccgagtggccatggcatgtgtggccctgtctgcttatcctcggaaggtgacagcgattccctgtgtagctctcacatgcacaggggcccatggactcttcagtctggagggtcctgggcctcctgacagcaataaataatttcgttggcc

SEQ ID NO: 15 - DNA, 2808 bp, Homo sapiens
ggcggagctgggcgaattaaagcacatggtgatgagtttccgggtgtctgagctccaggtgcttcttggctttgctggccggaacaagagtggacggaagcacgagctcctggccaaggctctgcacctcctgaagtccagctgtgcccctagtgtccagatgaagatcaaagagctttaccgacgacgctttccccggaagaccctggggccctctgatctctcccttctctctttgccccctggcacctctcctgtaggctcccctggtcctctagctcccattcccccaacgctgttggcccctggcaccctgctgggccccaagcgtgaggtggacatgcacccccctctgccccagcctgtgcaccctgatgtcaccatgaaaccattgcccttctatgaagtctatggggagctcatccggcccaccacccttgcatccacttctagccagcggtttgaggaagcgcactttacctttgccctcacaccccagcaagtgcagcagattcttacatccagagaggttctgccaggagccaaatgtgattataccatacaggtgcagctaaggttctgtctctgtgagaccagctgcccccaggaagattattttccccccaacctctttgtcaaggtcaatgggaaactgtgccccctgccgggttaccttcccccaaccaagaatggggccgagcccaagaggcccagccgccccatcaacatcacacccctggctcgactctcagccactgttcccaacaccattgtggtcaattggtcatctgagttcggacggaattactccttgtctgtgtacctggtgaggcagttgactgcaggaacccttctacaaaaactcagagcaaagggtatccggaacccagaccactcgcgggcactgatcaaggagaaattgactgctgaccctgacagtgaggtggccactacaagtctccgggtgtcactcatgtgcccgctagggaagatgcgcctgactgtcccttgtcgtgccctcacctgcgcccacctgcagagcttcgatgctgccctttatctacagatgaatgagaagaagcctacatggacatgtcctgtgtgtgacaagaaggctccctatgaatctcttatcattgatggtttatttatggagattcttagttcctgttcagattgtgatgagatccaattcatggaagatggatcctggtgcccaatgaaacccaagaaggaggcatctgaggtttgccccccgccagggtatgggctggatggcctccagtacagcccagtccaggggggagatccatcagagaataagaagaaggtcgaagttattgacttgacaatagaaagctcatcagatgaggaggatctgccccctaccaagaagcactgttctgtcacctcagctgccatcccggccctacctggaagcaaaggagtcctgacatctggccaccagccatcctcggtgctaaggagccctgctatgggcacgttgggtggggatttcctgtccagtctcccactacatgagtacccacctgccttcccactgggagccgacatccaaggtttagatttattttcatttcttcagacagagagtcagcactatggcccctctgtcatcacctcactagatgaacaggatgcccttggccacttcttccagtaccgagggaccccttctcactttctgggcccactggcccccacgctggggagctcccactgcagcgccactccggcgccccctcctggccgtgtcagcagcattgtggcccctgggggggccttgagggaggggcatggaggacccctgccctcaggtccctctttgactggctgtcggtcagacatcatttccctggactgagttccctggattatggaaacttcgctgtcccccaacactgagcaagtatgctgtggagtcccaaccccagctactctgatccctctgggggctctggccaagggccagacagaccttcacagatgcctacttttggcctcatctctgcctgacaaggccagcacccaaagggttaatatttaacctctttttaaggacactggggtctgtttctggaaatgttctttagatggtggcacattcctttgggtatgttaacctaggcagtgggaggcaaatgggatggtatgtgagctaggagaagggctgaaccctcagccttgactatgtctagagcctcttggggaaggggcacctctcttgaaccccaaatgctctctcttcttattacccaaacccatggctctatttcttcttcacatccattgtctcttcatgtctattccattcccttcggccaaacagacaggtggaaaaactgagacaggcagtttcagagatggacagagaactttattttggattgtggatgtggacttttttgtacataaataagaaaaaccaaaatactccaaagatgacttcccctgcctcctactccagtatgacagaggaggatgtaaggccttagccatgatctgcaggggtctgggagtcaggcccggcctattgcttgggtctctctctatttatatatctaagttcacagtgtttcttattccccctaagcttctagaggctcatggccctgtagttaggcctggctcattctgcacctttccagggaggtggaaggaccctgtgccctccttcccaatcttctttttcaggctcgccaaggcctaggacctatgttgtaattttactttttatttctaaagttgtagtgaagctctcacccataataaaggttgtgaatgttc

SEQ ID NO: 16 - DNA, 2333 bp, Homo sapiens
acgagcagcgacgcccctggtcccacagataccactgcctcccgccctttcgctcctcggccgcgcaatgggcacccgcgacgacgagtacgactacctctttaaagttgtccttattggagattctggtgttggaaagagtaatctcctgtctcgatttactcgaaatgagtttaatctggaaagcaagagcaccattggagtagagtttgcaacaagaagcatccaggttgatggaaaaacaataaaggcacagatatgggacacagcagggcaagagcgatatcgagctataacatcagcatattatcgtggagctgtaggtgccttattggtttatgacattgctaaacatctcacatatgaaaatgtagagcgatggctgaaagaactgagagatcatgctgatagtaacattgttatcatgcttgtgggcaataagagtgatctacgtcatctcagggcagttcctacagatgaagcaagagcttttgcagaaaagaatggtttgtcattcattgaaacttcggccctagactctacaaatgtagaagctgcttttcagacaattttaacagagatttaccgcattgtttctcagaagcaaatgtcagacagacgcgaaaatgacatgtctccaagcaacaatgtggttcctattcatgttccaccaaccactgaaaacaagccaaaggtgcagtgctgtcagaacatctaaggcatttctcttctcccctagaaggctgtgtatagtccatttcccaggtctgagatttaaatatatttgtaattcttgtgtcacttttgtgttttattacttcatacttatgaatttttccatgtcctaagtcttttgattttagctttataaaatcatccacttctcccgaatgactgcagctttttttcatgctatggcttcactagccttagtttaataaactgaatgtttggattcctcagttattgtttacttttcatcatggaagcctgtcactgtatgtaggacataatagaacttatcacttgaagctcagcctattggtcttgatcaaatcaaactaagaagaccttagaaataagctaccattttgccacagagcagcttataggtaatacactcttctctcagtgcagtgtacatttccacaaatctaagaattgccctataaacatagcagggattttgagagcttgaaaattttccattattctggacatgaatttctaaaatgccttaataggtttatgtagttgagtaaattttgttttttaatttttgtaagcatcaaagttgattagagaggggggcactttttctggagaattctcttagtaaacacaaaagattgttacggtttcattagtagtatggttgtggggccataagttaaacagtgctgcctggtaggctgggaactgaagagacttgtggtattccatctcgggtgcctctgttggcaatgatcaggcagcccaaaagatttaaatgatctataataatttccaagcggtagattatgtggcattttattgctcaggcaataattggtttaatgctggtagtgtcaaattttgaagtgttaattttgtcttaggaccttccagtaagtgaaatacaacctagttttatcaccatatccaccagcaggcatccataattattttaacaatgctaatatttgagttttgcagtatattatagaataatatagtccagttaaatctttggtttcagtatgtctgaagagtacagtgagaggttaattttctgctcaagtggtaccacttaaaggcatgtattcttttagtatgtaaaatgaaatagtaccttgagtttaaatagaatgcatttaggcattgtagagatctgaaatagttttcttccactgcgttgttgaaatcaatgaagcaattagtttctcattcagaaatgtgcacactaatatttagttttgctttctcgtggataatattaagcacttactctgcagtttctggaagttgtgtcaactgcagtgatactattcaggatggtgggaaatccccaaaaatatgtatcttttggcttgcttagattactatatttcatagttaatcttttgtctcttgcggtgctcatgatgtgtggggcacacggaaggcattgctgtagtcagtcattttggttttcttctatagccattttattattttagtgtattagttatgaagataatattatctatttgtaaattgctactttgtattttatgcatgctctgtaatttgatttttttttagttattgatttggattatattcacattctaataaacagttatagggggatt

SEQ ID NO: 17 - DNA, 4982 bp, Homo sapiens
gcggcgagcgcctgtcaagcagtgggaggaaaaagcgcttggagccaccagcccactcttaagcgaaactcgcctggaaaacaccccgcctcccagcagagtttctgccggtttctttagaagggtggtgcggaaaagcaacttttgggaacttgaaggagtggggtgtggggcgaagtggaaagagcacacaacgcgaccccactcagtccccgcttgggactcccatcccggcatcaggcgtagtgggcggtccgggccagggccagggccagagcggcggcggcgaggccatgccgagggcccgtcggccgcagttccccgtccggcctcgcgggggcgccggcgctgccgatcaggtgaccgagggcccgttcggggctgcgggaagcggcctcgttctcagccgccggagacgccgccgccgccgccgccgccgccacacctagtggagcagccggggaaggcggctcgttgggggctggggcggagagcgccgggggtgggggcggaagggcggcgggcggaaggcaggaggctgccggggcgcgggctgctgcgggagaaggggctccgaggagtccgccgcggctcgctctgtcgccggcgcgggattggggcgcgagggccatgggcgcgctctcctaaggcggaggtcgcgggcgggaggggaggaggcccgagagaggctgctgcgaaggccgcgggcccgtgactgggcgcgaggcggccggcggcggcggcggcaccagcaccaccatgtcgcgctcagtgctgcagcccagtcagcagaagctggcggagaagctcaccatcctcaacgaccggggcgtcggcatgctcacccgcctctacaacatcaagaagcaaggacaagtttggaaggcatgtggagaccccaaggcaaaaccatcctatcttatcgacaaaaacctggaatctgctgtgaaattcatagtcagaaaattccctgctgtagaaacccgcaacaacaatcaacagcttgcacaactacagaaagaaaaatcagagattctgaaaaatctggcattatattacttcacatttgtagatgttatggaatttaaggaccatgtttgtgaattgctgaatactattgacgtttgccaagtcttctttgatattactgtaaactttgatttaacaaagaactacttagatttaattataacctatacaacactaatgatactgctgtctcgaattgaagaaaggaaggcaatcattggattatacaactatgcccatgaaatgactcatggagcaagtgacagagaatacccacgccttggccagatgattgtggattatgaaaaccctttaaagaagatgatggaagaatttgtaccccatagcaagtctctttcagatgcactaatttctcttcaaatggtatatcctcgaaggaatctttcagctgaccagtggagaaatgcccagttattgagcctcatcagtgcacctagtacaatgcttaatccagcacagtccgacactatgccttgtgaatacctctctttggatgcaatggaaaagtggattatctttggctttattttgtgccatgggatcctaaatactgacgctacagcactgaacctttggaaactagctcttcaaagtagctcttgcctctctctctttcgggatgaagttttccacattcacaaagctgcagaagacttatttgtaaacatacgaggctataataaacgtattaatgacataagagaatgcaaggaggcagccgtgtcacatgctggttcaatgcacagagaaagacgcaagtttttaagatctgcactgaaggaattggctactgtcctctctgatcaacctggattgctaggtcccaaggcactttttgtttttatggcattatcctttgcccgtgatgaaatcatctggctacttcgtcatgcagataacatgccaaagaagagtgcagacgactttatagataagcacattgctgaattaatattttacatggaagaacttagagcacatgtgaggaaatacggacctgtaatgcagaggtattacgtgcagtacctttctggctttgatgctgttgtcctcaatgaactcgtgcagaatctttctgtttgccctgaagatgaatcaatcatcatgtcctcttttgttaacactatgacttccctaagtgtaaaacaagttgaagatggggaagtatttgatttcagaggaatgagattagattggtttaggttacaggcatatactagtgtctcaaaggcttcacttggccttgcagatcacagagaacttggaaagatgatgaatacaataatttttcatacaaaaatggtagattccttggtggaaatgttggtggaaacatcagatctctccatattttgtttttatagtcgtgcttttgagaagatgtttcaacagtgtttggagttaccctctcaatcaagatactcaattgcatttccactactttgcactcattttatgagttgcacgcatgaactatgtccagaagagcgacatcatattggagatcgcagtctttccttatgtaatatgttcctagatgaaatggccaaacaagctcgaaatctcatcactgatatttgcacagaacagtgtacccttagtgaccagttgctacccaagcattgtgccaaaactatcagtcaagcagtgaataagaaatcaaaaaagcagactggtaagaaaggggaacctgaaagggagaaaccaggtgttgagagcatgaggaaaaacaggctggttgtgaccaaccttgataaattgcacactgcactttctgagttatgcttctctataaattatgtaccaaacatggtggtatgggaacatacctttaccccacgagaatatttgacttctcatctggaaatacgctttaccaagtcaattgttgggatgactatgtataatcaagccacacaggaaattgcaaaaccttcagaacttctaacaagtgtaagagcatacatgaccgtactccagtcaatagaaaactatgtgcagattgatattacaagagtatttaataatgtgcttcttcaacaaacacaacatttagacagtcatggagagccaaccattacaagtctatacacaaattggtatttggaaactttgttacgacaagtcagcaatggccatatagcatattttcctgcaatgaaagcgtttgtgaacttacctacagaaaatgaattaacattcaatgcagaggaatattctgacatatcagaaatgaggtcattatcagaactactaggcccatatggtatgaagtttctaagtgaaagccttatgtggcatatttcatcacaagttgctgaacttaagaaacttgtggtggagaatgttgatgtgttaacacaaatgaggaccagctttgacaaaccagaccagatggctgcactgtttaaaagattatcatctgttgacagtgtcttgaagaggatgacaataattggtgtaattttatccttccgatcattggcacaagaagcacttagagatgtgttatcctaccacattccttttcttgtaagttcaattgaagattttaaggatcacattccaagggaaactgatatgaaggttgcaatgaatgtgtatgagttatcatcagctgccggattaccttgtgagattgatcctgcattggtcgtagctctttcttcacaaaaatcggaaaacattagtccagaagaagagtataaaattgcctgccttctcatggtgtttgtggcagtttctttgccaacactggccagtaatgtgatgtctcagtacagccctgctatagaagggcattgcaacaacatacattgcttggccaaagccatcaaccagattgctgcagctttgtttacaattcacaaaggaagcattgaagaccgtcttaaagaatttctggcgcttgcatcctccagtctactgaaaattggccaggagacagataaaactacaacaagaaatagagaatctgtttatttactgctagatatgattgtacaagaatctccattccttacaatggatcttttggaatcttgttttccttatgtcttgctgagaaatgcataccatgctgtctacaaacaaagtgttacatcttctgcataaaattacctacttaatcaagataagcacgcatttttgttgccttggttttacctgtagactgtggaactattttaccttaagacctgaaaaagttttgtggattataaatttctttcatacggttgtattttctgatcattggtttcttaatatggttgtactacagtatacttggttgatttaggttgcacattcactgaattcactgagattattcctataattttaaagtatcatttatttgaaaaacatacattatcaacatgtttttgatatttgataatgaaaaaaatctttgcttgtttatttctgaaaaagaactgtatttagtgattattttagatagtgatattatagcattcatctgtgtgtaaattatttcatatagggaagagttctgatctgtacctatggttcttattgaaaacaacattggatgtgcatttctgtgatgttatgaatacatttctactttattttgaaacatttgccaaactaaatactgtaacactgtataacatttaaaaatgttaaagaactgcttagtattagaagcagatcatttcccaaaattctaagagcagcagcatatgttgttgcttgtataaagcctagcgataatttttagactaacttccatggtgccctgttggcattagcactaccattgtaccctgctgtataataaacaatcttagacatttatcaactgttgatacaaatgttagtccctaaccactttttatatatgttttaaatttttgaaattcaagtgtaccttccataacataaaataaacactagactgtatcacactt

SEQ ID NO: 18 - DNA, 474 bp, Homo sapiens
ttttttttgctggtgccttaactgagtcatcttttttccattccattcatgaaagcaacataagaaagccaagagtgaaaggggtaaaagatcacagtataaaggtcttcggtgtgtccttcaaagatttacacaacattgtcctaaagggaagtcacagcagcttagctgtttctcacagatcagagaggatggtggggcagccaggagtcatcagtaaacccaggtgagcagtgcaggactgaatgtcgctgtccacttgcaggtgggagtccatgtggagggtgctccttcttgtttctcattgggacggtgactgtgtatagtggaaagcacagagccaatgagggacaggatggctgtggtggagggaaaacagtgcagggctgctctgtctctctgtcctgtcccgttaggactggtgggccaccacaggtttcgcgaaggtgtggctggccattcctttcctcgcgt

SEQ ID NO: 19 - DNA, 2091 bp, Homo sapiens
gaattccacctgcttcagtttgtgctgacaatcatccagaccagctgcggggtcatctggccgtgcacattccctcttggttggttgtatttccagattggatacatgatttccctgattgctctcttcacaaacttctacattcagacctacaacaagaaaggggcctcccgaaggaaagaccacctgaaggaccaccagaatgggtccatggctgctgtgaatggacacaccaacagcttttcacccctggaaaacaatgtgaagccaaggaagctgcggaaggattgaagtcaaagaattgaaaccctccaaaccacgtcatctgattgtaagcacaatatgagttgtgccccaatgctcgttaacagctgctgtaactagtctggcctacaatagtgtgattcatgtaggacttctttcatcaattcaaaacccctagaaaacgtatacagattatataagtagggataagatttctaacatttctgggctctctgacccctgcgctagactgtggaaagggagtattattatagtatacaacactgctgttgccttattagttataacatgataggtgctgaattgtgattcacaatttaaaaacactgtaatccaaacttttttttttaactgtagatcatgcatgtgattgtaaatgtaaacttgtacaatgttgttatggtagagaaacacacatgccttaaaatttaaaaagcagggcccaaagcttattagtttaaattagggtatgtttcaagtttgtattaatttgtaatagctctgtttagaaaaaatcaaagaccatgatttatgaaactaatgtgacataatttccagtgacttgttgatgtgaaatcagacacggcaccttcagttttgtactattggctttgaatcaagcaggctcaaatctagtggaacagtcagtttaactttttaacagatcttatttttttattttgagtgccactattaatgtaaaaaggggggggctctacagcagtcgtgatgaaacttaaatatatattctttgtcctcgagattttaggaagggtgtagggtgagtaggccatttttaatttctgaagtgctaagtgtttttatacagcaaacaaaaagtcaattttgctttccaccagtgcgagagaggatgtatacttttcaagagagatgattgcctatttaccgtttgacagagtcccgtagatgagcaatggggaactggttgccagggtctaaatttggattgatttatgcactgttatctgttttgacacagatttccttgtaaaatgtgcctagtttaccaaaattaacaaagggggggaaaggaccttagaactttttaaggtaaaatcaaatatagctacagcataagagaatcgagaaatttgatagaggtaacttgtttaatgtaaatctaatagtacttgtaatttctttctgcttagaatctaaagatgtgtttagaacctcttgtttaaaaataatagactgcttatcataaaatcacatctcacacatttgaggcagtggtcaaacaggtaaagcctatgatgtgtgtcattttaaagtgtcggaatttagcctctgaataccttctccattgggggaaagatattcttggaaccactcatgacatatcttagaaggtcattgacaatgtataaactaattgttggtttgatatttatgtaaatatcagtttaccatgctttaattttgcacattcgtactatagggagcctattggttctctattagtcttgtgggttttctgtttgaaaaggagtcatggcatctgtttacatttaccttatcaaacctagaatgtgtatatttataaatgtatgtcttcattgctaggtactaatttgcagatgtctttacatatttcaatacagaaactataacattcaatagtgtgctgtcaaagtgtgcttagctcacctggatatacctacattgttaaatgtctaaacagtaatcattaaaacatttttgattaaaaaaaaaaaaaaaaaaaaaaaaaaaaaaaaaaaaaaaaaaaaaaaaaaaaaaaaaaaaaaaaaaaaaaaaaaaaaa

SEQ ID NO: 20 - DNA, 1435 bp, Homo sapiens
ggcgcggctccgctctcggctggggttcgtcactgggcgcgggatttggccgccgcggggctccggagccgctcgctcccgacacggctcacgatgcgcggcgacagggccggcgggggccccgtgctccagttcactaactgccggatcctgcgcggagggaaactgctcagggaggatctgtgggtgcgcggaggccgcatcttggacccagagaagctgttctttgaggagcggcgcgtggccgacgagcggcgggactgcgggggccgcatcttggctcccggattcatcgacgtgcagatcaacggtggatttggtgttgacttctctcaagccacggaggacgtgggttcgggggttgccctcgtggcccgggagatcctgtcgcacgggtcacctccttctgccccaccctggtcacttcccaccggagtttatcacaaggttgttcctcagatccctgtgaagagtggtggtccccatgggcaaggggtcctcgggctgcacctggagggccccttcatcagccgggagaagcggggcacgcaccccgaggcccacctccgctccttcgaggccgatgccttccaggacttgctggccacctacgggcccctggacaatgtccgcatcgtgacgctggccccagagttgggcgtagcacgagtactccgggcgctgacggcgtggatctgcgtgtccctagggcactcagtggctgacctgcgggcggcagaggatgctgtgtggagcggcaccttcatcacccacctcttcaacgccatgctgcctttccaccaccgcgacccaggcatcgtggggctcctgaccagcgaccggctgcccgcaggccgctgcatcttctatgggatgattgcagatggcacgcacaccaaccccgccgccctgcggatcgcccaccgtgcccatccccaggggctggtgctggtcaccgatgccatccctgccttgggcctgggcaacggccggcacacgctgggacagcaggaagtggaagtggacggtctgacggcctacgtggcaggcaccaagacgctgagtggcagcatagccccaatggacgtctgtgtccggcacttcctgcaggccacaggctgcagcatggagtcggccctggaggctgcatccctgcaccccgcccagttgctggggctggagaagagtaaggggaccctggactttggtgctgacgcagacttcgtggtgctcgacgactcccttcacgtccaggccacctacatctcgggtgagctggtgtggcaggcggacgcagctaggcagtgacaaggacctcggctgagaggacacctggccgcagcgggatgccatcagggccgggtggttggggagctggtctccagggagtgagtcgggagccctgctggattgatgcccagcgctgtgcggccgc

SEQ ID NO: 21 - DNA, 2771 bp, Homo sapiens
  misc_feature (2658),(2660),(2753): n is a, c, g or t
gtactcgccacggcacccaggctgcgcgcacgcggtcccggtgtgcagctggagagcgagcggccaccgggagcccccggcacagcccgcgcccgccccgcaggagcccgcgaagatgccccggcgcagcctgcacgcggcggccgtgctcctgctggtgatcttaaaggaacagccttccagcccggccccagtgaacggttccaagtggacttattttggtcctgatggggagaatagctggtccaagaagtacccgtcgtgtgggggcctgctgcagtcccccatagacctgcacagtgacatcctccagtatgacgccagcctcacgcccctcgagttccaaggctacaatctgtctgccaacaagcagtttctcctgaccaacaatggccattcagtgaagctgaacctgccctcggacatgcacatccagggcctccagtctcgctacagtgccacgcagctgcacctgcactgggggaacccgaatgacccgcacggctctgagcacaccgtcagcggacagcacttcgccgccgagctgcacattgtccattataactcagacctttatcctgacgccagcactgccagcaacaagtcagaaggcctcgctgtcctggctgttctcattgagatgggctccttcaatccgtcctatgacaagatcttcagtcaccttcaacatgtaaagtacaaaggccaggaagcattcgtcccgggattcaacattgaagagctgcttccggagaggaccgctgaatattaccgctaccgggggtccctgaccacacccccttgcaaccccactgtgctctggacagttttccgaaaccccgtgcaaatttcccaggagcagctgctggctttggagacagccctgtactgcacacacatggacgacccttcccccagagaaatgatcaacaacttccggcaggtccagaagttcgatgagaggctggtatacacctccttctcccaagtgcaagtctgtactgcggcaggactgagtctgggcatcatcctctcactggccctggctggcattcttggcatctgtattgtggtggtggtgtccatttggcttttcagaaggaagagtatcaaaaaaggtgataacaagggagtcatttacaagccagccaccaagatggagactgaggcccacgcttgaggtccccggagctcccgggcacatccaggaaggaccttgctttggaccctacacacttcggctctctggacacttgcgacacctcaaggtgttctctgtagctcaatctgcaaacatgccaggcctcagggatcctctgctgggtgcctccttgccttgggaccatggccaccccagagccatccgatcgatggatgggtgcatctcagaccaagcagcaggaattcaaagctgcttgctgtaactgtgtgagattgtgaagtggtctgaattctggaatcacaaaccaagccatgctggtgggccattaatggttggaaaacactttcatcggggctttgccagagcgtgctttcaagtgtcctggaaagtctgctgcttctccaagctttcagacaagaatgtgcactctctgcttagtttgcttgggaaactcaacttctttcctctggagacggggcatctccctctgatttccttctgctatgacaaaacctttaatctgcaccttacaactcggggacaaatgggacaggaaggatcaagttgtagagagaaaaaagaaaacaagagatatacattgtgatatattagggacactttcacagtcctgtcttctggatcacagacactgcacagaccttagggaatggcaggttcaagttccacttcttggtggggatgagaagggagagagagctagagggacaaagagaatgagaagacatggatgatctgggagagtctcagtttggaatcagaattggaatcacattctgtttatcaagccataatgtaaggacagaataatacaatattaagtccaaatccaacctcctgtcagtggagcagttatgttttatactctacagattttacaaataatgaggctgttccttgaaaatgtgttgttgctgtgtcctggaggagacatgagttccgagatgacccaatctgcctttgaatctggaggaaataggcagaaacaaaatgactgtagaacttattctctgtaggccaaatttcatttcagccacttctgcaggatccctactgccaacctggaatggagacttttatctacttctctcttctctgaagatgtcaaatcgtggtttagatcaaatatatttcaagctataaaagcaggaggttatctgtgcagggggctggcatcatgtatttaggggcaagtaataatggaatgctactaagatactccatattcttccccgaatcaacacagacagtttctgacaggcgcaactcctccattttcctcccgcaggtgagaaccctgtggagatgagtcagtgccatgactgagaaggaaccgacccctagttgagagcaccttgcagttccccgagaactttctgattcacagtctcattttgacagcatgaaatgtcctcttgaagcatagctttttaaatatctttttccttctactcctccctctgactctaagaattctntnttctggaatcgcttgaacccaggaggcggaggttgcagtaagccaaggtcatgccactgcactctagcctgggtgacagagcgagactccatntcaaaaaaaaaaaaaaaa

SEQ ID NO: 22 - DNA, 264 bp, Homo sapiens
  misc_feature (235), (246), (247): n is a, c, g or t
gatctaatttatttttccatttatgtacagtttcttaatgtaaatttaaaagatatttgttgtgccctgctctgtaaacattcccttaatgcttatttgcatgaaagacttttgggttctatttgtttgtactatgtagctgtaagctgggtttgggtgtctaccttttctgtagtttctaacttttttaatttgttaatattctaaatacgtgttagaatcatgctttttatancctactttccnnatcccattatctcttta

SEQ ID NO: 23 - DNA, 2428 bp, Homo sapiens
cccacgcgtccggtctttgatgaagttgatacagatcaatctggtgtcttgtctgacagagaaatccgaacactggctaccagaattcacgaactgccgttaagtttgcaggatttgacaggtctggaacacatgctaataaattgctcaaaaatgcttcctgctgatatcacgcagctaaataatattccaccaactcaggaatcctactatgatcccaacctgccaccggtcactaaaagtctagtaacaaactgtaaaccagtaactgacaaaatccacaaagcatataaggacaaaaacaaatataggtttgaaatcatgggagaagaagaaatcgcttttaaaatgattcgtaccaacgtttctcatgtggttggccagttggatgacataagaaaaaaccctaggaagtttgtttgcctgaatgacaacattgaccacaatcataaagatgctcagacagtgaaggctgttctcagggacttctatgaatccatgttccccataccttcccaatttgaactgccaagagagtatcgaaaccgtttccttcatatgcatgagctgcaggaatggagggcttatcgagacaaattgaagttttggacccattgtgtactagcaacattgattatgtttactatattctcattttttgctgagcagttaattgcacttaagcggaagatatttcccagaaggaggatacacaaagaagctagtcccaatcgaatcagagtatagaagatcttcatttgaaaaccatctacctcagcatttactgagcattttaaaactcagcttcacagagatgtctttgtgatgtgatgcttagcagtttggcccgaagaaggaaaatatccagtaccatgctgttttgtggcatgaatatagcccactgaccaggaattatttaaccaacccactgaaaacttgtgtgttgagcagctctgaactgattttacttttaaagaatttgctcatggacctgtcatcctttttataaaaaggctcactgacaagagacagctgttaatttcccacagcaatcattgcagactaactttattaggagaagcctatgccagctgggagtgattgctaagaggctccagtctttgcattccaaagccttttgctaaagttttgcactttttttttttcatttcccatttttaagtagttactaagttaactagttattcttgcttctgagtataacgaattgggatgtctaaacctatttttatagatgttatttaaataatgcagcaatatcacctcttattgacaatacctaaattatgagttttattaatatttaagactgtaaatggtcttaaaccactaactactgaagagctcaatgattgacatctgaaatgctttgtaattattgacttcagcccctaagaatgctatgatttcacgtgcaggtctaatttcaaagggctagagttagtactacttaccagatgtaattatgttttggaaatgtacatattcaaacagaagtgcctcattttagaaatgagtagtgctgatggcactggcacattacagtggtgtcttgtttaatactcattggtatattccagtagctatctctctcagttggtttttgatagaacagaggccagcaaactttctttgtaaaaggctggttagtaaattattgcaggccacctgtgtctttgtcatacattcttcttgctgttgtttagtttgttttttttcaaacaaccctctaaaaatgtaaaaaccatgtttagcttgcagctgtacaaaaactgcccaccagccagatgtgaccctcaggccatcatttgccaatcactgagaattagtttttgttgttgttgttgttgttgtttttgagacagagtctctctctgttgcccaggctggagtgcagtggcgcaatctcagctcactgcaacctccgcctcccgggttcaagcagttctgtctcagccttctgagtagctgggactacaggtgcatgccaccacaccctgctaatttttgtatttttagtagagacgggggttccaccatattggtcaggcttatcttgaactcctgacctcaggtgatccacctgcctctgcctcccaaagtgctgagattacaggcataagccagtgcacccagccgagaattagtatttttatgtatggttaaaccttggcgtctagccatattttatgtcataatacaatggatttgtgaagagcagattccatgagtaactctgacaggtattttagatcatgatctcaacaatattcttccaaaatggcatacatcttttgtacaaagaacttgaaatgtaaatactgtgtttgtgctgtaagagttgtgtatttcaaaaactgaaatctcataaaaagttaaatttttgtctgaaaaaaaaaaaaaaaaa

SEQ ID NO: 24 - DNA, 1743 bp, Homo sapiens
agcagctgtcagggaacctccgccggagtcgaatttacgtgcagctgccggcaaccacaggttccaagatggtttgcgggggcttcgcgtgttccaagaactgcttgtgcgccctcaacctgctttacaccttggttagtctgctgctaattggaattgctgcgtggggcattggcttcgggctgatttccagtctccgagtggtcggcgtggtcattgcagtgggcatcttcttgttcctgattgctttagtgggtctgattggagctgtaaaacatcatcaggtgttgctatttttttatatgattattctgttacttgtatttattgttcagttttctgtatcttgcgcttgtttagccctgaaccaggagcaacagggtcagcttctggaggttggttggaacaatacggcaagtgctcgaaatgacatccagagaaatctaaactgctgtgggttccgaagtgttaacccaaatgacacctgtctggctagctgtgttaaaagtgaccactcgtgctcgccatgtgctccaatcataggagaatatgctggagaggttttgagatttgttggtggcattggcctgttcttcagttttacagagatcctgggtgtttggctgacctacagatacaggaaccagaaagacccccgcgcgaatcctagtgcattcctttgatgagaaaacaaggaagatttcctttcgtattatgatcttgttcactttctgtaattttctgttaagctccatttgccagtttaaggaaggaaacactatctggaaaagtaccttattgatagtggaattatatatttttactctatgtttctctacatgtttttttctttccgttgctgaaaaatatttgaaacttgtggtctctgaagctcggtggcacctggaatttactgtattcattgtcgggcactgtccactgtggcctttcttagcatttttacctgcagaaaaactttgtatggtaccactgtgttggttatatggtgaatctgaacgtacatctcactggtataattatatgtagcactgtgctgtgtagatagttcctactggaaaaagagtggaaatttattaaaatcagaaagtatgagatcctgttatgttaagggaaataccaaattcccaattttttttggtctttttaggaaagatgtgttgtggtaaaaagtgttagtataaaaatgataatttacttgagtcttttatgattacaccaatgtattctagaaatagttatgtcttaggaaattgtggtttaatttttgacttttacaggtaagtgcaaaggaaaagtggtttcatgaaatgttctaatgtataataacatttaccttcagcctccatcagaatggaacgagttttgagtaatcaggaagtatatctatatgatcttgatattgttttataataatttgaagtctaaaagactgcatttttaaacaagttagtattaatgcgttggcccacgtagcaaaaagatatttgattatcttaaaaattgttaaataccgttttcatgaaagttctcagtattgtaacagcaacttgtcaaacctaagcatatttgaatatgatctcccataatttgaaattgaaatcgtattgtgtggctctgtatattctgttaaaaaattaaaggacagaaacctttctttgtgtatgcatgtttgaattaaaagaaagtaatggaagaattgtaaaaaaaaaaaaaaaaaaaaaa

SEQ ID NO: 25 - DNA, 1412 bp, Homo sapiens
cggggtcgtccgcaaagcctgagtcctgtcctttctctctccccggacagcatgagcttcaccactcgctccaccttctccaccaactaccggtccctgggctctgtccaggcgcccagctacggcgcccggccggtcagcagcgcggccagcgtctatgcaggcgctgggggctctggttcccggatctccgtgtcccgctccaccagcttcaggggcggcatggggtccgggggcctggccaccgggatagccgggggtctggcaggaatgggaggcatccagaacgagaaggagaccatgcaaagcctgaacgaccgcctggcctcttacctggacagagtgaggagcctggagaccgagaaccggaggctggagagcaaaatccgggagcacttggagaagaagggaccccaggtcagagactggagccattacttcaagatcatcgaggacctgagggctcagatcttcgcaaatactgtggacaatgcccgcatcgttctgcagattgacaatgcccgtcttgctgctgatgactttagagtcaagtatgagacagagctggccatgcgccagtctgtggagaacgacatccatgggctccgcaaggtcattgatgacaccaatatcacacgactgcagctggagacagagatcgaggctctcaaggaggagctgctcttcatgaagaagaaccacgaagaggaagtaaaaggcctacaagcccagattgccagctctgggttgaccgtggaggtagatgcccccaaatctcaggacctcgccaagatcatggcagacatccgggcccaatatgacgagctggctcggaagaaccgagaggagctagacaagtactggtctcagcagattgaggagagcaccacagtggtcaccacacagtctgctgaggttggagctgctgagacgacgctcacagagctgagacgtacagtccagtccttggagatcgacctggactccatgagaaatctgaaggccagcttggagaacagcctgagggaggtggaggcccgctacgccctacagatggagcagctcaacgggatcctgctgcaccttgagtcagagctggcacagacccgggcagagggacagcgccaggcccaggagtatgaggccctgctgaacatcaaggtcaagctggaggctgagatcgccacctaccgccgcctgctggaagatggcgaggactttaatcttggtgatgccttggacagcagcaactccatgcaaaccatccaaaagaccaccacccgccggatagtggatggcaaagtggtgtctgagaccaatgacaccaaagttctgaggcattaagccagcagaagcagggtaccctttggggagcaggaggccaataaaaagttcagagttcattggatgtc

SEQ ID NO: 26 - DNA, 480 bp, Homo sapiens
cagtcctgagctgcgtcccggagcccacggtggtcatggctgccagagcgctctgcatgctggggctggtcctggccttgctgtcctccagctctgctgaggagtacgtgggcctgtctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcagaatgcaccttctgaggcacctccagctgcccccggccgggggatgcgaggctcggagcacccttgcccggctgtgattgctgccaggcactgttcatctcagcttttctgtccctttgctcccggcaagcgcttctgctgaaagttcatatctggagcctgatgtcttaacgaataaaggtcccatgctccacccgaggacagttcttcgtgcctg

SEQ ID NO: 27 - DNA, 126 bp, Homo sapiens
gatctttcataaagcagaatggtatgtatcggattgttttaatgttatatattggattgtattcgatgttacaaaaccaatattctatggagaatgaaaaaaataaaggactaaattaactggaaa

(19) United States
(12) Patent Application Publication
(10) Pub. No.: US 2005/0260572 A1
(43) Pub. Date: Nov. 24, 2005

(54) Method of predicting cancer

(76) Inventors: Kikuya Kato, Nishifunabashi, Hirakata-shi, Osaka (JP); Kyoko Iwao, Kasuga, Ibaraki-shi, Osaka (JP); Shinzaburo Noguchi, Miyakojima-ku, Osaka-shi, Osaka (JP); Ryo Matoba, Takeshirodai, Sakai-shi, Osaka (JP)

Correspondence Address: Reed Smith, Suite 1400, 3110 Fairview Park Drive, Falls Church, VA, 22042, US

(21) Appl. No.: 10/276,233

(22) PCT Filed: Mar. 7, 2002
(86) PCT No.: PCT/JP02/02153

§ 371 Date: Feb. 6, 2003

(30) Foreign Application Priority Data
Mar. 14, 2001 (JP) 2001-73063
Apr. 6, 2001 (JP) 20001-108503
Aug. 2, 2001 (JP) 2001-234807

(51) Int. Cl. C12Q 1/68; G06F 19/00; G01N 33/48; G01N 33/50

(52) U.S. Cl. 435/6; 702/20

(57) ABSTRACT

The invention relates to a cancer predicting method and a drug design method. Specifically, the invention relates to a method for predicting cancer which is useful for the genetic diagnosis for evaluating the malignancy of cancer. The invention also relates to a method for designing a drug based on the result of the above prediction method.

### TECHNICAL FIELD

The present invention relates to a method for predicting cancer and a drug design method. Particularly, the present invention relates to a cancer prediction method useful in genetic diagnosis for evaluating the malignancy of cancer. The invention also relates to a drug design method utilizing the results of the above prediction method.

### BACKGROUND ART

Various solid cancers such as breast cancer and colon cancer have different grade of malignancy depending on the individual case. As the various degrees of malignancy of individual cases require different methods of treatment, predicting prognosis is extremely important. Currently, cancer prognosis is performed by e.g. image analysis such as CT and X-ray, pathologic analysis such as tissue typing, and analysis utilizing a tumor marker. For example, CEA is well known as a molecular tumor marker for breast and colon cancers. This marker is not quite satisfactory for cancer diagnosis, however, because of its low sensitivity for early cancer and because in many cases detection of the cancer is possible only after the cancer is at an advanced stage. In addition, various methods of predicting cancer malignancy have been developed, but they only provide partial correlation with malignancy and their prediction results have not been satisfactory.

Recently, thanks to technologies such as DNA chips, it has become possible to systematically analyze the expression patterns of genes. As a result, it looks more likely than ever that cancer malignancy can be predicted on the basis of gene expression patterns.

On the other hand, it has been revealed that cancer is a disease caused by genetic abnormalities. In the field of clinical medicine, attention is being focused on genetic diagnosis of cancer based on a search for the responsible genes and detection of their abnormalities. Such genetic diagnosis of cancer is in great demand as a means of predicting the risks resulting from cancer, so that cancer can be prevented or treated in early stages.

### DISCLOSURE OF THE INVENTION

The object of the invention is to provide a method for predicting cancer and a drug design method.

The present inventors, after extensive work with a view to achieving the above objective, have succeeded in predicting cancer based on the result of multivariate analysis of expression levels of genes obtained from a primary cancerous lesion and thus have succeeded in completing the invention.

The invention provides a method for classifying cancer which comprises the steps of:

(a) collecting genes from specimens and measuring an expression level of the genes;

(b) selecting at least one of the measured genes;

(c) performing a multivariate analysis on the measurements of expression level for the selected genes; and

(d) classifying the specimens into groups with similar gene expression patterns by using the result of multivariate analysis as an indicator.

The present invention also provides a method for predicting cancer which comprises the steps of:

(a) collecting genes from specimens and measuring an expression level of the genes;

(b) selecting at least one of the measured genes;

(c) performing a multivariate analysis on the measurements of expression level for the selected genes;

(d) classifying the specimens into groups with similar gene expression patterns by using the result of multivariate analysis as an indicator; and

(e) predicting the state of cancer based on the result of classification.

The prediction method may include steps of determining an expression pattern characteristic of a particular state of cancer and comparing it with the expression patterns of genes collected from a cancer specimen on which cancer prediction is to be performed.

The states of cancer include at least one selected from the group consisting of the presence or absence of cancer, malignancy of cancer, presence or absence of metastasis of cancer, and presence or absence of recurrence of cancer. Metastasis of cancer includes lymph node metastasis, and recurrence includes early recurrence.

Examples of the selected genes include those of gene group I containing nucleotide sequences 1-27 from Table 1, those of gene group II containing nucleotide sequences 28-153 of Table 2, and those of gene group III containing nucleotide sequences 154-289 of Table 3. The selected genes may also include combinations of at least one gene selected from the group consisting of gene group I containing nucleotide sequences 1-27 of Table 1, gene group II containing nucleotide sequences 28-153 of Table 2, and gene group III containing nucleotide sequences 154-289 of Table 3, and at least one gene other than those of gene groups I, II and III.

One example of specimen classification employs a hormone receptor-positive group and/or a hormone receptor-negative group as an indicator. One example of the hormone receptor is estrogen receptor.

Examples of cancer include breast cancer, stomach cancer, esophageal cancer, oral cancer, colon cancer, rectal cancer, anal cancer, pancreatic cancer, lung cancer, renal cancer, bladder cancer, ovarian cancer, uterine cancer, skin cancer, melanoma, central nervous tumor, peripheral nervous tumor, gum cancer, pharyngeal cancer, maxillary and jowl cancer, liver cancer, prostate cancer, leukemia, multiple myeloma, and malignant limphoma. Particularly, breast cancer and colon cancer are preferable.

Multivariate analysis can be performed by cluster analysis.

The present invention further provides a drug design method, comprising designing a drug for suppressing the expression of a gene that is expressed in a specimen whose state of cancer has been predicted to be at high-risk by the above prediction method. Examples of such gene include genes having nucleotide sequences 4, 7 and 20 of Table 1, genes having nucleotide sequences 28, 29, 31, 32, 35, 43, 49-53, 67, 70, 72, 73, 75-79, 81, 84, 86-92, 94-99, 104-111, 113, 114, 117, and 122-153 from Table 2, and genes having nucleotide sequences 155, 162, 163, 167-169, 171, 172, 174, 175, 177-180, 188, 190, 193, 198, 211, 222, 242-253, 255-257, 259-261, 263 and 265 from Table 3, or combinations thereof. One example of the drug for suppressing the expression of the above-mentioned gene is an antisense nucleic acid. The present invention further provides a drug design method, comprising designing a drug for enhancing the expression of a gene that is expressed in a specimen whose state of cancer has been predicted to be at high-risk by the above prediction method. Such genes include genes having nucleotide sequences 1, 2, 3, 5, 6, 8, 9, 10, 11, 12, 13, 14, 15, 16, 17, 18, 19 and 21 of Table 1, genes having nucleotide sequences 30, 33, 34, 36-62, 44-48, 54-66, 68, 69, 71, 74, 80, 82, 83, 85, 93, 100-103, 112, 115, 116 and 118-121 of Table 2, and genes having nucleotide sequences 154, 156-161, 164-166, 170, 173, 176, 181-187, 189, 191, 192, 194-197, 199-210, 212-221, 223-241, 254, 258, 262, 264 and 266-289 of Table 3, or combinations thereof. One example of the drug for enhancing the expression of any of the above genes is a targeting vector in which the particular gene has been incorporated.

The invention also provides a program for having a computer function as a cancer state predicting system comprising a means of analyzing the expression level of a gene collected from a primary cancerous lesion and a means of identifying the state of the cancer by using the result of analysis as an indicator.

The present invention further provides a computer-readable recording medium in which is stored a program for having a computer function as a cancer state predicting system comprising a means of analyzing the expression level of a gene collected from a primary cancerous lesion and a means of identifying the state of the cancer by using the result of analysis as an indicator.

The present invention will be hereafter described in detail. This application claims priority from Japanese Patent Application Nos. 2001-73063 filed on Mar. 14, 2001, 2001-108503 filed on Apr. 6, 2001, and 2001-234807 filed on Aug. 2, 2001. The present specification includes part or all of the contents as disclosed in the specification and/or drawings of the above applications.

The method of the present invention is characterized in that specimens are classified into several groups according to differences in expression patterns of a particular gene, wherein an expression pattern characteristic of a state of cancer is determined based on the result of classification. The method of the invention is summarized in FIG. 1. First, a number of specimens, both normal and cancerous, are collected (see FIG. 1(e)), and the expression level of genes deriving from a primary cancerous lesion in these specimens is measured (see FIG. 1(f)). The measurement of the gene expression levels in these specimens is performed for all of the genes selected by searching the literature, for example (see FIG. 1(c)). Next, genes useful for multivariate analysis are selected from the genes for which the expression level has been measured. The selected genes are subjected to data analysis such as multivariate analysis (see FIG. 1(g)), and the specimens are classified into a small number of groups of similar expression patterns. The number of indicators for the classification into the small number of groups (i.e. the number of the classified groups) is not more than 20, preferably not more than 10, and more preferably 2. For example, the number is two when the groups consist of a hormone receptor-positive group and a hormone receptor-negative group (however, there might be cases where there is another group created in which the positive and negative groups are mixed). Based on the result of analysis, an expression pattern characteristic of a particular state of cancer is determined (see FIG. 1(h)). The state of cancer can be predicted by comparing the expression pattern in a specimen whose state of cancer is to be predicted with the patterns of the classified groups. It is also possible to determine, based on the results of classification, the malignancy of cancer or whether or not metastasis is occuring. Thereafter a gene specific to a different state of cancer, such as malignancy, can be determined by using the result of expression pattern analysis in the cancer state predicting method, and a drug can be designed for controlling the expression of the gene or the activity of a gene product.

1. Quantification of Gene Expression

In order to determine gene expression level, RNA is isolated from a specimen. Isolation of a gene may be performed by any known method. Examples of the method include a method by which cDNA is synthesized from an RNA prepared by a guanidine-isothiocyanate method. Examples of the gene to be isolated and determined include a gene derived from a primary cancerous lesion and a gene encoding an immunoglobulin, and many other genes thought to be relevant to cancer prediction may be selected by searching the literature.

Gene expression data may be obtained by any desired method, such as competitive PCR, TaqMan PCR, and Northern blot technique.

(1) Competitive PCR

Competitive PCR is a method for determining gene expression levels by amplifying identical genes contained in a plurality of samples in the same reaction system. One example of the competitive PCR method is an adaptor-tagged competitive PCR method (see FIG. 2). In this method, a different kind of adaptor sequence is added to each one of the identical cDNAs contained in at least two kinds of samples. The cDNAs are amplified after the individual samples containing cDNAs to which these adaptor sequences were added are mixed, and then quantitative ratios of the amplified cDNAs are determined (see Japanese Patent No. 2905192). In the following, the adaptor-tagged competitive PCR method is briefly described.

Initially, at least two kinds of samples containing the cDNA to be determined are prepared (two kinds of samples are taken as an example for simplicity). After cleaving the cDNAs in the samples with a specific restriction enzyme, an adaptor is added to the cleavage site. The adaptor refers to an oligonucleotide designed such that it can discriminate different cDNAs with the different oligonucleotides when amplified. The adaptors are designed as double-stranded such that they can bind to the restriction enzyme cleavage site of the cDNA. The adaptors may be designed such that the length of the adaptor added to the cDNA in one sample is different from that of the adaptor tagged to the cDNA in the other sample. Alternatively, the adaptors may be designed such that at least one restriction enzyme-recognition site is contained in both the adaptor added to the cDNA in one sample and the adaptor added to the cDNA in the other sample. Further alternatively, the adaptors may be designed such that the adaptor added to the cDNA in one sample is different in nucleotide sequence from that added to the cDNA in the other sample (examples A and B are shown in FIG. 2). These adaptors may be chemically synthesized. They may also be labeled by a fluorescent label or a radioisotope.

The samples each containing the adaptor tagged cDNA are mixed (preferably in equal amounts). Then, amplification is performed using the cDNAs in these samples as templates, by polymerase chain reaction (PCR), for example. After amplification, amplified products are detected using an automated sequencer (from e.g. Pharmacia) or an image scanner (from e.g. Molecular Dynamics). In the case where a radioisotope has been used, the detection is carried out using a densitometer or the like. As shown at the bottom of FIG. 2, the cDNAs can be quantitatively determined based on differences in the signal level from the labels in the sequences to which different adaptors were added.

(2) TaqMan PCR Method

TaqMan PCR is a method whereby amplification reaction and fluorescence intensity are measured simultaneously in a mixed reaction system (reaction tube) of a template, primer and labeled probe, so that fluorescent reporter dye released from a specific probe hybridized to the template is detected in real time and the PCR products are automatically analyzed by a computer connected to the detector (also called a real-time PCR method). This real-time detection PCR method is known, and apparatuses and kits for the method are commercially available. Thus, the present invention can employ such commercially available apparatuses or kits to detect gene expression (examples of the kits include TaqMan PCR kit and TaqMan EZ RT-PCR kit from ABI).

(3) Northern Blotting

The Northern blotting is a method for analyzing the size or amount of gene transcription products (mRNA) being expressed in a cell. Total RNA or mRNA extracted from the cell is subjected to denatured agarose gel electrophoresis, transferred onto a nylon or nitrocellulose membrane and fixed on the membrane. By hybridizing the membrane to a target gene, the size and existing amount of the mRNA of the gene are analyzed.

Apparatuses and kits for performing the Northern blotting are also commercially available. Examples include the Message Maker reagent set and a full-automatic electrophoresis blotting system (from Labimap).

(4) Detection by PCR Method

Primers for the detection of the above-mentioned gene, that is a forward primer (sense primer) and a reverse primer (anti-sense primer) for PCR, are designed and synthesized based on the nucleotide sequence of the gene so that, taking into account the amplification efficiency of PCR, the size of amplified fragment may be about 50 to 200 bp. The reverse primer is designed such that it is complementary to the based sequence. The primers may be designed by selecting a plurality of desired sequences from one or more different kinds of sequences taken from the above-mentioned based sequences.

The above primers may be chemically synthesized in a conventional manner, such as by using a DNA automatic synthesizer from Applied Biosystems (the same applies to nucleotide synthesis below). In the case of adaptor-tagged competitive PCR, only the reverse primer needs to be designed toward the poly (A) from the adaptor-tagged site.

(5) Probe

The probes used in the present invention may comprise an oligonucleotide labeled by binding a fluorescent reporter dye and a fluorescent quencher dye thereto.

The oligonucleotide portion of the gene detection probe may be designed on the basis of all or part of the sequence of the gene used in the present invention. Further, the oligonucleotide can be used that is capable of hybridizing to all or part of the nucleotide sequence of the gene under stringent conditions and that has a sequence of at least 15 contiguous nucleotides.

“Stringent conditions” refer to conditions where, in the case of using the TaqMan probe in real-time PCR, the probe and the primers simultaneously associate or hybridize with the template DNA. More specifically, the conditions include the use of a conventional buffer solution at temperatures of 60 to 65° C. Accordingly, the probe used in the present invention may have a mutation such as deletion, substitution or addition in one or more (e.g. from 1-10) nucleotides, as long as the probe can hybridize to the DNA to be detected under the above-mentioned stringent conditions. Further, the probe sequence may have approximately 1-10% of mismatchs to the nucleotide sequence of the region to be hybridized, as long as it can hybridize under the stringent conditions.

As a result of fluorescent resonance energy transfer, the fluorescence intensity of the above fluorescent reporter dye is suppressed when it is bound to the same probe as that to which the fluorescent quencher dye is bound. The intensity is not suppressed when the fluorescent reporter dye is not bound to the same probe as that of the fluorescent quencher dye. The fluorescent reporter dye may be preferably be the fluorescein type, such as FAM (6-carboxy-fluorescein). The fluorescent quencher dye may be preferably of the rhodamine type, such as TAMRA (6-carboxy-tetramethyl-rhodamine). These fluorescent dyes are known and readily available. The binding sites of the fluorescent reporter dye and fluorescent quencher dye are not particularly limited. Typically, the fluorescent reporter dye binds to one end (preferably the 5′-end) of the oligonucleotide of the probe, and the fluorescent quencher dye binds to the other end.

2. Selection of the Gene

From among the genes of which expression levels were measured as described above, genes useful for the multivariate analysis to be described later are selected. “Useful genes” refer to those genes that are selected from among the genes from which expression levels have been measured above and which can be discriminated or classified according to differences in the expression level when multivariate analysis is performed as described below. In the present invention, initially, genes that are to be used for quantitative determination of expression for the purpose of predicting prognosis, for example, are selected. The genes used for the quantitative determination of expression are ones useful in classifying cancer specimens and which satisfy predetermined criteria, and are selected depending on the type of cancer that is to be predicted. In the present invention, the types of genes used for predicting prognosis, for example, are not particularly limited as long as they are expressed in a primary cancerous lesion. Types of cancer include breast cancer, stomach cancer, esophageal cancer, oral cancer, colon cancer, rectal cancer, anal cancer, pancreatic cancer, lung cancer, renal cancer, bladder cancer, ovarian cancer, uterine cancer, skin cancer, melanoma, central nervous tumor, peripheral nervous tumor, gum cancer, pharyngeal cancer, maxillary and jowl cancer, liver cancer, prostate cancer, leukemia, multiple myeloma, and malignant limphoma. A gene expressed in at least one type of cancer selected from the above group can be used. The method for selecting the gene varies depending on the type of cancer. For example, the method includes selection by: the expression of hormone receptor; the result of other cluster analyses; presence or absence of lymph node metastasis; presence or absence of recurrence; prognostic factors; and/or tissue type. An example of metastasis is lymph node metastasis. An example of recurrence is early recurrence, which is a systemic recurrence within two years after an operation. Thus, by selecting genes useful in classifying tumor tissue and performing multivariate analysis, tumor tissue can be classified into groups according to characteristics of cancer development based on expression profile.

When predicting for breast cancer, a gene determining whether or not hormone receptors are expressed, particularly estrogen receptors, is preferable, in that it plays an important role in determining the nature of the breast cancer. When predicting for colon cancer, it is preferable to classify genes into a statistically significant number of clusters by performing cluster analysis according to the expression pattern of the genes, and select a group of genes belonging to a cluster relating to metastasis and/or prognosis factors. Clusters relating to metastasis and/or prognosis factors can be selected by performing principal component analysis or hierarchical cluster analysis on each of the above-classified clusters for their expression patterns, classifying samples according to expression patterns, and then examining the relationship between this classification and the prognosis and/or prognosis factors. In this case, therefore, all of the genes are subjected to multivariate analysis in advance in order to select the genes useful for further multivariate analysis.

In the present invention, when classifying cancer specimens using the genes by which the presence or absence of expression of the above-mentioned estrogen receptors can be determined, the expression can be linked to metastasis or recurrence based on the different degrees of malignancy of the specimens. “Genes by which the presence or absence of estrogen receptor can be determined” refer to those genes by which the specimens can be classified into an estrogen receptor-positive group and an estrogen receptor-negative group, when determining the expression level of a gene isolated from a specimen, and performing multivariate analysis as described later. Specifically, a plurality of specimens (normal and cancerous tissues) are collected and reacted with an antibody against estrogen receptor to determine whether the specimens are positive or negative for the receptor. Based on the results of this determination and on those of the expression of the above genes, cluster analysis is performed so that genes are selected by which the specimens can be classified into estrogen receptor-positive and negative groups.

In the present invention, cancer specimens can be related to metastasis or recurrence based on differences in the degree of malignancy by classifying the specimens by cluster analysis using a gene group(s) belonging to the cluster relating to metastasis and/or prognosis factors.

In the selection of the genes, prior to selecting the genes on the basis of the above-mentioned predetermined criteria, the ratio of the variation of gene expression level in cancer specimens to the variation of gene expression level in normal specimens may be calculated, so that genes satisfying a predetermined criteria can be selected in advance.

Variation within subgroup (Vg) is expressed by the following equation:

$$Vg = {\sum }_{i = 1}^{p} {\sum }_{j = 1}^{q} {( Xi - \overline{Xj} )}^{2} \qquad ( I )$$

wherein \overline{Xj} is an average of the gene expression levels in each group, p is the number of genes, q is the number of groups, and Xi is the expression level of a gene. Thus, Vg is the sum of the square of the difference between each level and the average in the normal or cancer specimen group. The ratio may be suitably changed depending on some factors including the type of genes to be analysed, the number of cases, and the number of genes. However, the ratio is normally from 1.10 to 1.20, preferably not less than 1.18 (e.g. from 1.80 to 1.20).

In the case of breast cancer, for example, the selection of genes can be performed by applying the principle of analysis of variance to the presence or absense of expression of estrogen receptors. First, by setting the ratio of the variation within the normal specimen subgroup to that within the cancer specimen subgroup at 1.20, for example, 152 genes out of 2412 genes can be selected in advance. Next, for the tissue or cell samples in each case (e.g. blood, removed lesion, biopsy sample), the presence or absence of expression of the estrogen receptor is detected by using an antibody against the estrogen receptor in a conventional manner (ELISA or RIA, for example), and dividing the samples into an estrogen receptor-positive group and an estrogen receptor-negative group. Thereafter the ratio of the variation of expression level within each group (variation within subgroup) to the variation of all of groups (total variation) is calculated. Genes for which this ratio satisfies a predetermined criteria are selected.

The total variation (Vt) is expressed by the following equation:

$$Vt = {\sum }_{i = 1}^{p} {( Xi - \overline{Xt} )}^{2} \qquad ( II )$$

wherein Xi and p are as described above, and \overline{Xt} is an average of the gene expression levels in total all the samples. Thus, Vt indicates the sum of the squares of the difference between each value of the gene expression level and the total average of the positive and negative groups.

The variation within subgroup (Vg) is as described above, namely it is expressed by the following equation:

$$Vg = {\sum }_{i = 1}^{p} {\sum }_{j = 1}^{q} {( Xi - \overline{Xj} )}^{2} \qquad ( I )$$

wherein \overline{Xj} is an average of the gene expression levels within each group, q is the number of groups, and Xi and p are as described above. Thus, Vg is the sum of the square of the difference between the detected level of each sample and the average of the positive or negative group.

The ratio may be suitably changed depending on some factors including the type of genes to be analyzed, the number of cases, and the number of genes. However, the ratio (total variation/variation within subgroup) is normally from 1.10 to 1.20, preferably not less than 1.18 (e.g. from 1.18 to 1.20).

In the present invention, when the indicator is such that the specimens are divided into the estrogen receptor-positive (ER+) group and negative (ER−) group, 27 types of genes (gene group I) can be selected as shown in numbers 1 to 27 in the “No.” column of Table 1 below. These genes are genes by which, when subjected to multivariate analysis, the presence or absence of expression of the estrogen receptor can be discriminated.

TABLE 1

| | Gene | | | | |
|---|---|---|---|---|---|
| No. | name | A.N. (EST) | A.N. | Contents of gene | SEQ ID NO: |
| 1 | GS1176 | AI092005 | S82616 | p6 = cytochrome c oxidase | 1 |
| | | | | subunit VIc | |
| | | | | homolog/COSVIc/prostatic | |
| | | | | carcinoma | |
| | | | | upregulated gene [human, | |
| | | | | prostate carcinoma cell line | |
| | | | | PC3, mRNA Partial, 261 nt]. | |
| 2 | GS1472 | AW249669 | L13850 | Homo sapiens hXBP-1 | 2 |
| | | | | transcription factor DNA. | |
| 3 | GS1957 | AA622242 | D90427 | Human mRNA for | 3 |
| | | | | zinc-alpha2-glycoprotein | |
| | | | | precursor, complete cds. | |
| 4 | GS2307 | AI141674 | X14583 | Human mRNA for | 4 |
| | | | | immunoglobulin (Ig) | |
| | | | | lambda-chain. | |
| 5 | GS2471 | AI369429 | | | 5 |
| 6 | GS2632 | AI891146 | Z97630 | Human DNA sequence from | 6 |
| | | | | clone RP3-466N1 on | |
| | | | | chromosome 22q12-13: | |
| | | | | Contains the H1F0 (H1 | |
| | | | | histone family member 0) | |
| | | | | gene, 2-amino-3-ketobutyrate | |
| | | | | CoA ligase (nuclear gene | |
| | | | | encoding mitochondrial | |
| | | | | protein), and GALR3 (gal). | |
| 7 | GS2828 | AI923193 | X51755 | Human | 7 |
| | | | | lambda-immunoglobulin | |
| | | | | constant region complex | |
| | | | | (germline). | |
| 8 | GS6601 | AI041822 | X60111 | H. sapiens mRNA for MRP-1. | 8 |
| 9 | GS6770 | AW052188 | | | 9 |
| 10 | GS6784 | AI570120 | U92544 | Human hepatocellular | 10 |
| | | | | carcinoma associated protein | |
| | | | | (JCL-1) mRNA, complete | |
| | | | | cds. | |
| 11 | GS690 | AI688954 | S70290 | Glutamine synthetase (human, | 11 |
| | | | | tumorous liver, mRNA | |
| | | | | Partial) | |
| 12 | GS7012 | | | | 12 |
| 13 | GS7116 | AA885475 | AF161540 | Homo sapiens HSPC055 | 13 |
| | | | | mRNA, complete cds. | |
| 14 | GS7264 | AI128061 | AF089747 | Homo sapiens | 14 |
| | | | | alpha-1-antichymotrypsin | |
| | | | | precursor, mRNA, partial cds. | |
| 15 | GS7288 | AI912299 | AB021868 | Homo sapiens PIAS3 mRNA | 15 |
| | | | | for protein inhibitor of | |
| | | | | activated STAT3, complete | |
| | | | | cds. | |
| 16 | GS7583 | AI800822 | AF000231 | Homo sapiens rab11a GTPase | 16 |
| | | | | mRNA, complete cds. | |
| 17 | GS7715 | AI126560 | AB011159 | Homo sapiens mRNA for | 17 |
| | | | | KIAA0587 protein, complete | |
| | | | | cds. | |
| 18 | GS6711 | AW005373 | | | 18 |
| 19 | GS7632 | AL119034 | AF111849 | Homo sapiens PRO0530 | 19 |
| | | | | mRNA, complete cds. | |
| 20 | GS7435 | AI092753 | AF132948 | Homo sapiens CGI-14 protein | 20 |
| | | | | mRNA, complete cds. | |
| 21 | GS7001 | AW051878 | AF037335 | Homo sapiens carbonic | 21 |
| | | | | anhydrase precursor (CA 12) | |
| | | | | mRNA, complete cds. | |
| 22 | GS7071 | | | | 22 |
| 23 | GS6703 | | AL359588 | DKFZp762B226 (nameless | 23 |
| | | | | complete cDNA) | |
| 24 | GS4353 | | AF120265 | Human tetraspan NET-6 | 24 |
| | | | | mRNA, complete cds. | |
| 25 | GS3006 | | M26326 | Human keratin 18 mRNA, | 25 |
| | | | | complete cds. | |
| 26 | GS3295 | | L15203 | Human secretory protein | 26 |
| | | | | (P1.B) mRNA | |
| 27 | GS2189 | | XM_002311 | Homo sapiens 126 bp | 27 |
| | | | | sequence | |

A.N.: Accession number.

In multivariate analysis, more than one desired gene out of gene group I can be selected in any combination. For example, genes indicated by Nos. 1-21 in the “No.” column of Table 1 should preferably be used. It is also possible to select one or more genes other than gene group I but for which expression levels have been measured and combine with one or more genes of gene group I. The genes other than those of gene group I may have characteristics which are totally different from or similar to those of the genes of gene group I. For example, genes encoding immunoglobulin or other genes may be selected.

In the case of colon cancer, for example, the genes can be selected by carrying out cluster analysis based on gene expression patterns and thus classifying the genes into a statistically significant number of clusters according to the gene expression patterns, thereby selecting a gene group belonging to a cluster preferable for multivariate analysis. In the present invention the cluster preferable for multivariate analysis is a cluster relating to, e.g., metastasis and/or prognostic factors. The cluster relating to the metastasis and/or prognostic factors can be selected by classifying the samples (specimens) of each of the above-classified clusters according to expression patterns by principal component analysis or hierarchical cluster analysis, and then using the relationship between this classification and the prognosis and/or prognostic factors as a reference or indicator.

In the present invention, the present inventors have found that 1536 genes relating to colon cancer could be classified by cluster analysis into 44 clusters, of which the cluster relating to metastasis was cluster No. 14, and the clusters relating to the prognostic factor were clusters Nos. 42-44. As the genes belonging to cluster No. 14, the 126 genes (referred to as gene group II) indicated by Nos. 28-153 in the “No.” column in Table 2 below can be selected and they could be used for multivariate analysis. As the genes belonging to cluster Nos. 42-44, the 136 genes indicated by Nos. 154-289 in the “No.” column in Table 3 below could be selected (“gene group III”) and they could be used for multivariate analysis. These genes are related to metastasis or prognosis when multivariate analysis is performed.

TABLE 2

| | Gene | | |
|---|---|---|---|
| No. | name | A.N. | Contents of gene |
| 28 | GS2695 | Y14737 | Homo sapiens mRNA for immunoglobulin lambda heavy chain. |
| 29 | GS169 | M23905 | Human MHC class II lymphocyte antigen (DPw4-alpha-1) gene. |
| 30 | GS846 | M13560 | Human Ia-associated invariant gamma-chain gene, exon 8, |
| | | | clones lambda-y(1, 2, 3). |
| 31 | GS2307 | X14583 | Human mRNA for Ig lambda-chain. |
| 32 | GS3628 | S49006 | Ig kappa {clone cYF.kappa} [human, mRNA Partial, 1209 nt]. |
| 33 | GS3813 | L12387 | Human sorcin (SRI) mRNA, complete cds. |
| 34 | GS3616 | AF072097 | Homo sapiens beta-2 microglobulin gene, complete cds. |
| 35 | GS1954 | Z11793 | H. sapiens mRNA for selenoprotein P. |
| 36 | GS3642 | L26165 | Human DNA synthesis inhibitor mRNA, complete cds. |
| 37 | GS2660 | X04412 | Human mRNA for plasma gelsolin. |
| 38 | GS690 | AL161952 | Homo sapiens mRNA; cDNA DKFZp434M0813 (from clone |
| | | | DKFZp434M0813); partial cds. |
| 39 | GS1864 | M36501 | Human alpha-2-macroglobulin mRNA, 3′ end. |
| 40 | GS4210 | X67698 | H. sapiens tissue specific mRNA. |
| 41 | GS1119 | AF207829 | Homo sapiens SCAN-related protein RAZ1 (RAZ1) mRNA, |
| | | | partial cds. |
| 42 | GS3516 | D14043 | Human mRNA for MGC-24, complete cds. |
| 43 | GS1329 | V01512 | Human cellular oncogene c-fos (complete sequence). |
| 44 | GS2960 | AF226869 | Homo sapiens RB-associated KRAB repressor (RBAK) mRNA, |
| | | | complete cds. |
| 45 | GS5569 | U14750 | Human connective tissue growth factor mRNA, partial cds. |
| 46 | GS1834 | X62320 | H. sapiens mRNA for epithelin 1 and 2. |
| 47 | L09159 | L09159 | Homo sapiens RHOA proto-oncogene multi-drug-resistance |
| | | | protein mRNA, 3′ end. |
| 48 | X05026 | X05026 | Human rho mRNA (clone 12). |
| 49 | GS3614 | AF021792 | Homo sapiens Bcl-X/Bcl-2 binding protein (BAD) mRNA, |
| | | | partial cds. |
| 50 | GS2704 | AA025421 | H. sapiens mRNA for HES1 protein |
| 51 | GS3295 | L15203 | Human secretory protein (P1.B) mRNA, complete cds. |
| 52 | GS4523 | X73685 | C. aethiops hsp70 mRNA. |
| 53 | GS2707 | AF054183 | Homo sapiens GTP binding protein mRNA, complete cds. |
| 54 | GS194 | AF217517 | Homo sapiens uncharacterized bone marrow protein BM041 |
| | | | mRNA, complete cds. |
| 55 | GS2723 | M35252 | Human CO-029. |
| 56 | GS3240 | M58485 | Human lysosomal membrane glycoprotein CD63 mRNA. |
| 57 | GS3529 | X04297 | Human mRNA for Na, K-ATPase alpha-subunit. |
| 58 | GS1054 | X75861 | H. sapiens TEGT gene. |
| 59 | GS6870 | D16562 | Human mRNA for ATP synthase gamma-subunit (L-type), |
| | | | complete cds. |
| 60 | GS1008 | M11353 | Human H3.3 histone class C mRNA, complete cds. |
| 61 | GS3048 | X61156 | H. sapiens mRNA for laminin-binding protein. |
| 62 | GS1458 | X54304 | Human mRNA for myosin regulatory light chain. |
| 63 | GS1756 | AK000841 | Homo sapiens cDNA FLJ20834 fis, clone ADKA02953, |
| | | | highly similar to AF115384 Homo sapiens LR8. |
| 64 | GS821 | M27891 | Human cystatin C (CST3) gene, exon 3. |
| 65 | GS3245 | M84643 | Macaca mulatta thioredoxin mRNA, complete cds. |
| 66 | GS2260 | U27143 | Human protein kinase C inhibitor-I cDNA, complete cds. |
| 67 | GS2991 | L07287 | Human ribosomal protein L26 (RPL26) gene exon 2, complete |
| | | | cds. |
| 68 | GS2789 | X04803 | Homo sapiens ubiquitin gene. |
| 69 | GS137 | L19739 | Homo sapiens metallopanstimulin (MPS1) mRNA, complete |
| | | | cds. |
| 70 | GS3565 | L25085 | Human Sec61-complex beta-subunit mRNA, complete cds. |
| 71 | AF077034 | AF077034 | Homo sapiens HSPC010 mRNA, complete cds. |
| 72 | GS3819 | AF117616 | Homo sapiens SOUL protein (SOUL) mRNA, complete cds. |
| 73 | GS3424 | AK000462 | Homo sapiens cDNA FLJ20455 fis, clone KAT05813. |
| 74 | GS4401 | U47414 | Human cyclin G2 mRNA, complete cds. |
| 75 | GS4568 | AL049963 | Homo sapiens mRNA; cDNA DKFZp564A132 (from clone |
| | | | DKFZp564A132). |
| 76 | GS6584 | J04611 | Human lupus p70 (Ku) autoantigen protein mRNA, complete |
| | | | cds. |
| 77 | GS4090 | U24704 | Human antisecretory factor-1 mRNA, complete cds. |
| 78 | GS2932 | X52317 | Human mRNA for histone H2A.Z. |
| 79 | GS2365 | Z49835 | H. sapiens mRNA for protein disulfide isomerase. |
| 80 | GS2495 | D00422 | Human sphingolipid activator proteins, mRNA, complete cds. |
| 81 | GS3021 | X01060 | Human mRNA for transferrin receptor. |
| 82 | GS3823 | X05344 | Human mRNA for cathepsin D from oestrogen responsive |
| | | | breast cancer cells. |
| 83 | GS983 | M30685 | Pan Troglodytes MHC class I protein mRNA (MHCPATRF1). |
| 84 | GS726 | X58536 | Human mRNA for HLA class I locus C heavy chain. |
| 85 | GS3409 | NM_001101 | Homo sapiens actin, beta (ACTB), smRNA. |
| 86 | GS7358 | M74817 | Human tropomyosin-1 (TM-beta) mRNA, complete cds. |
| 87 | GS3542 | D49400 | Homo sapiens mRNA for vacuolar ATPase, complete cds. |
| 88 | GS2965 | U90654 | Human zinc-finger domain-containing protein mRNA, partial |
| | | | cds. |
| 89 | GS1990 | X04481 | Human mRNA for complement component C2. |
| 90 | GS3222 | U44954 | Human NMDA receptor glutamate-binding chain (hnrgw) |
| | | | mRNA, partial cds. |
| 91 | GS697 | Z37166 | H. sapiens BAT1 mRNA for nuclear RNA helicase (DEAD |
| | | | family). |
| 92 | GS1353 | D50372 | Homo sapiens mRNA for myosin regulatory light chain, |
| | | | complete cds. |
| 93 | GS3621 | AC004938 | Homo sapiens clone DJ0971C03, complete sequence. |
| 94 | GS2907 | AA633993 | Cell division cycle 10 (homologous to CDC10 of S. cerevisiae |
| 95 | GS3383 | AK000070 | Homo sapiens cDNA FLJ20063 fis, clone COL01524. |
| 96 | GS3043 | M32306 | Human epithelial glycoprotein (EGP) mRNA, complete cds. |
| 97 | GS6968 | AF044221 | Homo sapiens HCG-1 protein (HCG-1) mRNA, complete cds. |
| 98 | GS2998 | D87667 | Human brain mRNA homologous to 3′UTR of human CD24 |
| | | | gene, partial sequence. |
| 99 | GS2752 | M29540 | Human carcinoembryonic antigen mRNA (CEA), complete cds. |
| 100 | GS3752 | AB018270 | Homo sapiens mRNA for KIAA0727 protein, partial cds. |
| 101 | GS3223 | AL133580 | Homo sapiens mRNA; cDNA DKFZp434N2072 (from clone |
| | | | DKFZp434N2072). |
| 102 | GS1264 | M21575 | Human cytochrome c oxidase COX subunit IV (COX IV) |
| | | | mRNA, complete cds. |
| 103 | GS201 | AB009010 | Homo sapiens mRNA for polyubiquitin UbC, complete cds. |
| 104 | GS3904 | Z35415 | H. sapiens gene encoding E-cadherin, exon 16. |
| 105 | GS3390 | U29091 | Human selenium-binding protein (hSBP) mRNA, complete cds. |
| 106 | GS2252 | X01683 | Human mRNA for alpha 1-antitrypsin. |
| 107 | GS3412 | J03544 | Human brain glycogen phosphorylase mRNA, complete cds. |
| 108 | GS2952 | AF007194 | Homo sapiens mucin (MUC3) mRNA, partial cds. |
| 109 | GS3116 | X91863 | H. sapiens Gpx2 gene. |
| 110 | GS3779 | M81600 | Human NAD(P)H: quinone oxireductase gene, exon 6. |
| 111 | GS1655 | J03746 | Human glutathione S-transferase mRNA, complete cds. |
| 112 | GS145 | M77234 | Human ribosomal protein S3a mRNA, complete cds. |
| 113 | GS2032 | AF104238 | Homo sapiens ADP-ribosylation factor 4 (ARF4) gene, |
| | | | exon 6 and complete cds. |
| 114 | GS133 | J04617 | Human elongation factor EF-1-alpha gene, complete cds. |
| 115 | GS7058 | U35048 | Human TSC-22 protein mRNA, complete cds. |
| 116 | GS2547 | X96752 | H. sapiens mRNA for L-3-hydroxyacyl-CoA dehydrogenase. |
| 117 | GS4723 | AF047470 | Homo sapiens malate dehydrogenase precursor (MDH) mRNA, |
| | | | nuclear gene encoding mitochondrial protein, complete cds. |
| 118 | GS243 | AB021288 | Homo sapiens mRNA for beta 2-microglobulin, complete cds. |
| 119 | GS3682 | AF151802 | Homo sapiens CGI-44 protein mRNA, complete cds. |
| 120 | GS2791 | D13629 | Human mRNA for KIAA0004 gene, complete cds. |
| 121 | GS7410 | AF075010 | Homo sapiens full length insert cDNA YI03D03. |
| 122 | GS1208 | U72512 | Human B-cell receptor associated protein (hBAP) |
| | | | alternatively spliced mRNA, partial 3′UTR. |
| 123 | GS7407 | AA485677 | Human zyxin related protein ZRP-1 mRNA, complete cds |
| 124 | GS3119 | NM_003641 | Homo sapiens interferon induced stransmembrane |
| | | | protein 1 (9-27) (IFITM1), mRNA. |
| 125 | GS988 | L08246 | Human myeloid cell differentiation protein (MCL1) mRNA. |
| 126 | U15173 | U15173 | Homo sapiens BCL2/adenovirus E1B 19 kD-interacting protein |
| | | | 2mRNA, complete cds. |
| 127 | GS2263 | AB002382 | Human mRNA for KIAA0384 gene, complete cds. |
| 128 | GS2848 | AC004258 | Homo sapiens chromosome 19, cosmid R33114, complete |
| | | | sequence. |
| 129 | GS2535 | AL096818 | Human DNA sequence from clone RP1-262C15 on chromosome |
| | | | 6q16.1-21. |
| | | | Contains the 3′ end of a novel gene, ESTs, STSs and GSSs, |
| | | | complete sequence. |
| 130 | GS3269 | AF113016 | Homo sapiens PRO1073 mRNA, complete cds. |
| 131 | L11910 | L11910 | Human retinoblastoma susceptibility gene exons 1-27, complete |
| | | | cds. |
| 132 | GS3644 | Z85986 | Human DNA sequence from clone 108K11 on chromosome 6p21 |
| | | | Contains SRP20 (SR protein family member), Ndr protein kinase |
| | | | gene similar to yeast suppressor protein SRP40, EST and GSS, |
| | | | complete sequence. |
| 133 | GS2973 | V00662 | H. sapiens mitochondrial genome. |
| 134 | GS2726 | AJ002190 | Homo sapiens cDNA for dihydroxyacetone |
| | | | phosphateacyltransferase (DAP-AT). |
| 135 | GS906 | AF182645 | Homo sapiens chondrosarcoma-associated protein 2 (CSA2) |
| | | | mRNA, complete cds. |
| 136 | GS3950 | L06070 | Human squalene synthetase (ERG9) mRNA, complete cds. |
| 137 | GS2524 | X71129 | H. sapiens mRNA for electron transfer flavoprotein beta subunit. |
| 138 | GS1768 | J04058 | Human electron transfer flavoprotein alpha-subunit mRNA, |
| | | | complete cds. |
| 139 | GS5905 | D13866 | Human mRNA for alpha-catenin, complete cds. |
| 140 | GS3741 | L08666 | Homo sapiens porin (por) mRNA, complete cds and truncated |
| | | | cds. |
| 141 | GS3426 | D21235 | Human mRNA for HHR23A protein complete cds. |
| 142 | GS2512 | BC005402 | Homo sapiens , clone MGC: 12543, mRNA, complete cds. |
| 143 | GS1662 | Y10211 | H. sapiens LAG-3 gene, promoter region. |
| 144 | GS3873 | D14662 | Human mRNA for KIAA0106 gene, complete cds. |
| 145 | GS261 | AF047439 | Homo sapiens unknown mRNA, complete cds. |
| 146 | GS3611 | NM_006793 | Homo sapiens peroxiredoxin 3s(PRDX3), nuclear gene encoding |
| | | | mitochondrial protein, smRNA. |
| 147 | GS273 | X59066 | Human mRNA for mitochondrial ATP synthase (F1-ATPase) |
| | | | alpha subunit. |
| 148 | GS242 | M81457 | Human calpactin 1 light chain mRNA, complete cds. |
| 149 | GS410 | M11146 | Human ferritin H chain mRNA, complete cds. |
| 150 | GS599 | M77233 | Human ribosomal protein S7 mRNA, 3′ end. |
| 151 | GS1042 | X87838 | H. sapiens mRNA for beta-catenin. |
| 152 | GS308 | Y00345 | Human mRNA for poly A binding protein. |
| 153 | GS3608 | X63753 | H. sapiens son-a mRNA. |

A.N.: Accession Number

TABLE 3

| | Gene | | |
|---|---|---|---|
| No. | name | A.N. | Contents of gene |
| 154 | GS2976 | X87342 | H. sapiens mRNA for human giant larvae homolog. |
| 155 | GS4595 | BC002507 | Homo sapiens , WD repeat domain 13, clone |
| | | | MGC: 1020, mRNA, complete cds. |
| 156 | GS3264 | X06347 | Human mRNA for U1 small nuclear RNP-specific |
| | | | A protein. |
| 157 | GS2141 | L35013 | Human spliceosomal protein (SAP 49) gene, |
| | | | complete cds. |
| 158 | GS3995 | NM_003379 | Homo sapiens villin 2 (ezrin)s(VIL2), mRNA. |
| 159 | GS4409 | AK001523 | Homo sapiens cDNA FLJ10661 fis, clone |
| | | | NT2RP2006106. |
| 160 | GS4687 | NM_019606 | Homo sapiens hypothetical proteinsFLJ20257 |
| | | | (FLJ20257), mRNA. |
| 161 | GS2984 | AJ000342 | Homo sapiens mRNA for DMBT1 6 kb transcript |
| | | | variant 1 (DMBT1/6 kb.1). |
| 162 | GS2891 | X74215 | H. sapiens mRNA for Lon protease-like protein. |
| 163 | GS4065 | AL050372 | Homo sapiens mRNA; cDNA DKFZp434A091 |
| | | | (from clone DKFZp434A091). |
| 164 | GS4782 | NM_004911 | Homo sapiens protein disulfidesisomerase related |
| | | | protein (calcium-binding protein, |
| | | | sintestinal-related) (ERP70), mRNA. |
| 165 | GS4735 | AF118224 | Homo sapiens matriptase mRNA, complete cds. |
| 166 | GS724 | AF077051 | Homo sapiens PTD001 mRNA, complete cds. |
| 167 | GS4072 | AF151806 | Homo sapiens CGI-48 protein mRNA, complete |
| | | | cds. |
| 168 | GS4682 | AF035959 | Homo sapiens type-2 phosphatidic acid |
| | | | phosphatase-gamma (PAP2-g) mRNA, complete |
| | | | cds. |
| 169 | GS3068 | M15205 | Human thymidine kinase gene, complete cds, |
| | | | with clustered Alu repeats in the introns. |
| 170 | GS2846 | X65614 | H. sapiens mRNA for calcium-binding protein |
| | | | S100P. |
| 171 | GS4185 | AJ011916 | Homo sapiens mRNA for hypothetical protein. |
| 172 | GS3154 | AL121673 | Human DNA sequence from clone RP11-305P22 |
| | | | on chromosome 20 Contains ESTs, STSs, GSSs and |
| | | | 7 CpG islands. |
| | | | Contains three novel genes and a novel gene for |
| | | | a helix-loop-helix DNA binding protein, complete |
| | | | sequence. |
| 173 | GS502 | X63423 | H. sapiens mRNA for delta-subunit of |
| | | | mitochondrial F1F0 ATP-synthase (clone #5). |
| 174 | GS4552 | D45370 | Human apM2 mRNA for GS2374 (unknown |
| | | | product specific to adipose tissue), complete cds. |
| 175 | GS5215 | M30952 | Orangutan 28S ribosomal RNA gene fragment. |
| 176 | GS2425 | AB023165 | Homo sapiens mRNA for KIAA0948 protein, |
| | | | complete cds. |
| 177 | GS4267 | NM_013440 | Homo sapiens pairedsimmunoglobulin-like |
| | | | receptor beta (PILR(BETA)), mRNA. |
| 178 | GS4832 | NM_001571 | Homo sapiens interferon regulatory factor 3 |
| | | | (IRF3), mRNA. |
| 179 | GS3249 | U22055 | Human 100 kDa coactivator mRNA, complete cds. |
| 180 | GS855 | AK001829 | Homo sapiens cDNA FLJ10967 fis, clone |
| | | | PLACE1000798. |
| 181 | GS765 | AJ238097 | Homo sapiens mRNA for Lsm5 protein. |
| 182 | L33075 | L33075 | Homo sapiens ras GTPase-activating-like protein |
| | | | (IQGAP1) mRNA, complete cds. |
| 183 | GS4482 | AF154502 | Homo sapiens quiescent cell proline dipeptidase |
| | | | (QPP) mRNA, complete cds. |
| 184 | GS4008 | NM_012426 | Homo sapiens splicing factor 3b, ssubunit 3, 130 kD |
| | | | (SF3B3), mRNA. |
| 185 | GS1540 | U19721 | Human peroxisomal targeting signal receptor 1 |
| | | | (PXR1) mRNA, complete cds. |
| 186 | GS2869 | AL023653 | Human DNA sequence from clone 753P9 on |
| | | | chromosome Xq25-26.1. Contains the gene coding |
| | | | for Aminopeptidase P (EC 3.4.11.9, |
| | | | XAA-Pro/X-Pro/Proline/Aminoacylproline |
| | | | Aminopeptidase) and a novel gene. Contains ESTs, |
| | | | STSs, GSSs and a gaaa repeat polymorphism, |
| | | | complete sequence. |
| 187 | GS4498 | AF151105 | Homo sapiens 3′-5′ exonuclease TREX1 mRNA, |
| | | | complete cds. |
| 188 | GS4263 | BC004925 | Homo sapiens , Similar to G protein-coupled |
| | | | receptor, family C, group 5, member C, clone |
| | | | MGC: 10304, mRNA, complete cds. |
| 189 | GS4198 | AK000453 | Homo sapiens cDNA FLJ20446 fis, clone |
| | | | KAT05231. |
| 190 | GS3749 | M98326 | Human transfer valyl-tRNA synthetase mRNA, 3′ |
| | | | end of cds. |
| 191 | D38122 | D38122 | Human mRNA for Fas ligand, complete cds. |
| 192 | R76314 | R76314 | Ras homolog gene family, member G (rho G) |
| 193 | GS2718 | U31201 | Human laminin gamma2 chain gene (LAMC2), |
| | | | exon 23 and flanking sequences, and complete cds. |
| 194 | GS3664 | L16862 | Homo sapiens G protein-coupled receptor kinase |
| | | | (GRK6) mRNA, complete cds. |
| 195 | GS4718 | Z14978 | H. sapiens mRNA for actin-related protein. |
| 196 | GS3193 | AL022240 | Human DNA sequence from clone 328E19 on |
| | | | chromosome 1q12-21.2. |
| | | | Contains a cyclophilin-like gene, a novel gene, |
| | | | ESTs, GSSs and STS, complete sequence. |
| 197 | GS3533 | X05231 | Human mRNA for collagenase (E.C. 3.4.24). |
| 198 | GS4112 | AF054178 | Homo sapiens CI-B14.5a homolog mRNA, |
| | | | complete cds. |
| 199 | GS4559 | NM_002085 | Homo sapiens glutathionesperoxidase 4 |
| | | | (phospholipid hydroperoxidase) (GPX4), smRNA. |
| 200 | GS3260 | D86966 | Human mRNA for KIAA0211 gene, complete cds. |
| 201 | GS3924 | AB002312 | Human mRNA for KIAA0314 gene, partial cds. |
| 202 | GS2867 | D84471 | Homo sapiens mRNA for phenylalanyl tRNA |
| | | | synthetase beta subunit, complete cds. |
| 203 | GS3014 | AL096737 | Homo sapiens mRNA; cDNA DKFZp434F152 |
| | | | (from clone DKFZp434F152). |
| 204 | GS4183 | AF068007 | Homo sapiens cell cycle-regulated factor p78 |
| | | | mRNA, complete cds. |
| 205 | GS4515 | AK000154 | Homo sapiens cDNA FLJ20147 fis, clone |
| | | | COL07954. |
| 206 | GS779 | AF069378 | Homo sapiens insulin-like growth factor II |
| | | | receptor (IGF2R) gene, exon 48 and partial cds. |
| 207 | GS4438 | NM_018475 | Homo sapiens TPA regulated locus (TPARL), |
| | | | mRNA. |
| 208 | GS4407 | AC005361 | Homo sapiens chromosome 16, cosmid clone |
| | | | 352F10 (LANL), complete sequence. |
| 209 | GS4452 | U71274 | Human mutant factor XII gene, exon 14 and partial |
| | | | cds. |
| 210 | GS3234 | X65024 | H. sapiens mRNA for xeroderma pigmentosum |
| | | | group C complementing factor (XP-C). |
| 211 | U37100 | U37100 | Homo sapiens aldose reductase-like peptide |
| | | | mRNA, complete cds. |
| 212 | GS3829 | AF042346 | Homo sapiens putative phenylalanyl-tRNA |
| | | | synthetase beta-subunit mRNA, complete cds. |
| 213 | GS1393 | AF094516 | Homo sapiens E1-like protein mRNA, complete |
| | | | cds. |
| 214 | GS4048 | AL110243 | Homo sapiens mRNA; cDNA DKFZp564B0482 |
| | | | (from clone DKFZp564B0482); complete cds. |
| 215 | GS3944 | AB003184 | Homo sapiens mRNA for ISLR, complete cds. |
| 216 | GS3235 | U31556 | Human transcription factor E2F-5 mRNA, |
| | | | complete cds. |
| 217 | GS3408 | Z18538 | H. sapiens encoding skin-derived |
| | | | antileukoproteinase. |
| 218 | GS3248 | NM_012262 | Homo sapiens heparan |
| | | | sulfates2-O-sulfotransferase (HS2ST1), mRNA. |
| 219 | GS4420 | AF060567 | Homo sapiens sushi-repeat protein (SRPUL) |
| | | | mRNA, complete cds. |
| 220 | GS3004 | Y12777 | Homo sapiens mRNA for acyl-CoA synthetase-like |
| | | | protein. |
| 221 | GS3310 | AB032903 | Homo sapiens GMPR2 mRNA for guanosine |
| | | | monophosphate reductase isolog, complete cds. |
| 222 | GS1333 | AK024628 | Homo sapiens cDNA: FLJ20975 fis, clone |
| | | | ADSU01705. |
| 223 | GS2948 | AF151908 | Homo sapiens CGI-150 protein mRNA, complete |
| | | | cds. |
| 224 | GS2124 | AB011128 | Homo sapiens mRNA for KIAA0556 protein, |
| | | | partial cds. |
| 225 | GS3751 | X17567 | H. sapiens RNA for snRNP protein B. |
| 226 | GS4173 | NM_004719 | Homo sapiens splicing factor's |
| | | | arginine/serine-rich 2, interacting protein |
| | | | (SFRS2IP), smRNA. |
| 227 | GS2765 | AF075589 | Homo sapiens MDA-MB-231 peripheral-type |
| | | | benzodiazepine receptor (PBR) mRNA, partial cds. |
| 228 | GS4811 | AL157435 | Homo sapiens mRNA; cDNA DKFZp434O0510 |
| | | | (from clone DKFZp434O0510). |
| 229 | GS3135 | M59371 | Human protein tyrosine kinase mRNA, complete |
| | | | cds. |
| 230 | GS4162 | X92762 | H. sapiens mRNA for tafazzins protein. |
| 231 | GS2744 | J02947 | Human extracellular-superoxide dismutase (SOD3) |
| | | | mRNA, complete cds. |
| 232 | GS4893 | NM_019027 | Homo sapiens hypothetical proteins(FLJ20273), |
| | | | mRNA. |
| 233 | AI341099 | AI341099 | Orf1 5′ to PD-ECGF/TP . . . orf2 5′ to PD-ECGF/TP |
| | | | [human, epidermoid carcinoma cell line A431, |
| | | | mRNA, 3 genes, 1718 nt] |
| 234 | GS1494 | AL133034 | Homo sapiens mRNA; cDNA DKFZp727K171 |
| | | | (from clone DKFZp727K171); partial cds. |
| 235 | GS3474 | D83174 | Human mRNA for collagen binding protein 2, |
| | | | complete cds. |
| 236 | GS2928 | NM_015140 | Homo sapiens KIAA0153 proteins(KIAA0153), |
| | | | mRNA. |
| 237 | GS4106 | AB020628 | Homo sapiens mRNA for KIAA0821 protein, |
| | | | complete cds. |
| 238 | GS4000 | AF055377 | Homo sapiens long form transcription factor |
| | | | C-MAF (c-maf) mRNA, complete cds. |
| 239 | GS3286 | Y07604 | H. sapiens mRNA for nucleoside-diphosphate |
| | | | kinase. |
| 240 | L11701 | L11701 | Human phospholipase D mRNA, complete cds. |
| 241 | GS3170 | L35240 | Human enigma gene, complete cds. |
| 242 | GS2892 | NM_004368 | Homo sapiens calponin 2 (CNN2), smRNA. |
| 243 | GS4015 | NM_005433 | Homo sapiens v-yes-1 Yamaguchi sarcoma viral |
| | | | oncogene homolog 1 (YES1), mRNA. |
| 244 | GS3588 | AF131848 | Homo sapiens clone 24922 mRNA sequence, |
| | | | complete cds. |
| 245 | GS4780 | AD001530 | Homo sapiens XAP-5 mRNA, complete cds. |
| 246 | GS4941 | NM_016380 | Homo sapiens differentiation-related protein dif13 |
| | | | (LOC51212), mRNA. |
| 247 | GS4945 | NM_016343 | Homo sapiens centromere protein Fs(350/400 kD, |
| | | | mitosin) (CENPF), mRNA. |
| 248 | GS4163 | AC007565 | Homo sapiens chromosome 19, cosmid R27656, |
| | | | complete sequence. |
| 249 | GS3387 | NM_013317 | Homo sapiens hT1a-1 (hT1a-1), smRNA. |
| 250 | GS3386 | NM_003337 | Homo sapiens ubiquitin-conjugating enzyme E2B |
| | | | (RAD6 homolog) (UBE2B), smRNA. |
| 251 | GS4946 | NM_002439 | Homo sapiens mutS (E. coli) homolog 3 (MSH3), |
| | | | mRNA. |
| 252 | GS3019 | NM_003348 | Homo sapiens ubiquitin-conjugating enzyme E2N |
| | | | (homologous to yeastsUBC13) (UBE2N), mRNA. |
| 253 | GS4022 | NM_002433 | Homo sapiens myelin oligodendrocyte |
| | | | glycoprotein (MOG), mRNA. |
| 254 | GS4947 | NM_018520 | Homo sapiens hypothetical protein PRO2268 |
| | | | (PRO2268), mRNA. |
| 255 | GS1341 | BC001002 | Homo sapiens , clone IMAGE: 3447696, mRNA, |
| | | | partial cds. |
| 256 | GS4512 | NM_005768 | Homo sapiens putative protein similar to nessy |
| | | | (Drosophila) (C3F), mRNA. |
| 257 | GS4501 | AF261689 | Homo sapiens DNA polymerase epsilon p17 |
| | | | subunit gene, complete cds. |
| 258 | GS6969 | AL022316 | Human DNA sequence from clone CTA-126B4 on |
| | | | chromosome 22q13.2-13.31 Contains two or three |
| | | | novel genes, ESTs, STSs, GSSs and a CpG Island, |
| | | | complete sequence. |
| 259 | GS6493 | NM_014925 | Homo sapiens KIAA1002 proteins(KIAA1002), |
| | | | mRNA. |
| 260 | GS715 | NM_020221 | Homo sapiens hypothetical |
| | | | proteinsDKFZp547I224 (DKFZp547I224), mRNA. |
| 261 | GS3002 | U50871 | Human familial Alzheimer's disease (STM2) gene, |
| | | | complete cds. |
| 262 | GS1102 | AL020995 | Human DNA sequence from clone RP1-117O3 on |
| | | | choromosome 1p33-34.3, |
| | | | complete sequence. |
| 263 | GS5239 | U93574 | Human L1 element L1.39 p40 and putative p150 |
| | | | genes, complete cds. |
| 264 | M15990 | M15990 | Human c-yes-1 mRNA. |
| 265 | GS7322 | AF119386 | Homo sapiens blood plasma glutamate |
| | | | carboxypeptidase precursor (PGCP) mRNA, |
| | | | complete cds. |
| 266 | GS3683 | AK026017 | Homo sapiens cDNA: FLJ22364 fis, clone |
| | | | HRC06575. |
| 267 | GS4288 | NM_020983 | Homo sapiens adenylate cyclase 6s(ADCY6), |
| | | | transcript variant 2, mRNA. |
| 268 | GS2715 | NM_016442 | Homo sapiens type 1 tumor necrosis factor |
| | | | receptor shedding aminopeptidase regulator |
| | | | (ARTS-1), mRNA. |
| 269 | GS4364 | NM_002339 | Homo sapiens lymphocyte-specific protein 1 |
| | | | (LSP1), mRNA. |
| 270 | GS3138 | M76378 | Human cysteine-rich protein (CRP) gene, exons 5 |
| | | | and 6. |
| 271 | GS3607 | U76713 | Human apobec-1 binding protein 1 mRNA, |
| | | | complete cds. |
| 272 | GS4976 | NM_014133 | Homo sapiens PRO0618 proteins(PRO0618), |
| | | | mRNA. |
| 273 | GS964 | AL136622 | (Homo sapiens mRNA; cDNA DKFZp564B172 |
| | | | from clone DKFZp564B172); complete cds. |
| 274 | GS4824 | AF150105 | Homo sapiens small zinc finger-like protein |
| | | | (TIM9b) mRNA, complete cds. |
| 275 | GS3217 | NM_000786 | Homo sapiens cytochrome P450, 51s(lanosterol |
| | | | 14-alpha-demethylase) (CYP51), mRNA. |
| 276 | GS3380 | AB018255 | Homo sapiens mRNA for KIAA0712 protein, |
| | | | complete cds. |
| 277 | GS4038 | L43619 | Homo sapiens polycystic kidney disease (PKD1) |
| | | | gene, exons 43-46. |
| 278 | GS4375 | AL035541 | Human DNA sequence from clone 718J7 on |
| | | | chromosome 20q13.31-13.33. Contains part of a |
| | | | gene for a novel protein, the PCK1 gene for |
| | | | soluble phosphoenolpyruvate carboxykinase 1, |
| | | | part of a novel gene similar to mouse DLM-1 |
| | | | (tumour stroma and activated macrophage protein), |
| | | | the 3′ end of the TMEPAI gene encoding an |
| | | | androgen induced 1b transmembrane protein |
| | | | (PMEPA1), two putative novel genes, a CpG |
| | | | island, ESTs, STSs and GSSs, complete sequence. |
| 279 | GS3847 | NM_017964 | Homo sapiens hypothetical proteinsFLJ20837 |
| | | | (FLJ20837), mRNA. |
| 280 | GS3289 | AL136897 | Homo sapiens mRNA; cDNA DKFZp434E248 |
| | | | (from clone DKFZp434E248); complete cds. |
| 281 | GS4702 | S79219 | metastasis-associated gene [human, highly |
| | | | metastatic lung cell subline Anip[937], mRNA |
| | | | Partial, 978 nt]. |
| 282 | GS4742 | NM_006468 | Homo sapiens polymerase (RNA) IIIs(DNA |
| | | | directed) (62 kD) (RPC62), mRNA. |
| 283 | GS2904 | D38594 | Human MTH1 gene for 8-oxo-dGTPase, exon4, |
| | | | complete cds. |
| 284 | GS4563 | U53347 | Human neutral amino acid transporter B mRNA, |
| | | | complete cds. |
| 285 | GS1178 | U01184 | Human homolog of D. melanogaster flightless-I |
| | | | gene product mRNA, partial cds. |
| 286 | GS4062 | AF183423 | Homo sapiens reticulocabin precursor mRNA, |
| | | | complete cds. |
| 287 | GS4394 | U43923 | Human transcription factor SUPT4H mRNA, |
| | | | complete cds. |
| 288 | GS2956 | U02619 | Human TFIIIC Box B-binding subunit mRNA, |
| | | | complete cds. |
| 289 | D26443 | D26443 | GLUT1(glucose transporter) |

A.N.: Accession Number

In multivariate analysis, more than one desired gene can be selected from gene group II and/or gene group III in any combination. For example, it is referable to use genes of No. 30, 33, 34, 36-42, 44-48, 54-66, 68, 69, 71, 74, 80, 82, 83, 85, 93, 100-103, 112, 115, 116 and/or 118-121 from Table 2, and/or genes of No. 155, 162, 163, 167-169, 171, 172, 174, 175, 177-180, 188, 190, 193, 198, 211, 222, 242-253, 255-257, 259-261, 263 and/or 265 from Table 3. Further, more than one gene, not from gene groups II or III but for which expression level has been measured, may be combined with the above gene(s). The genes other than genes of gene groups II and/or III may have characteristics which are totally different from or similar to those of the genes of gene groups II and/or III. For example, genes encoding immunoglobulin or other genes may be selected.

3. Multivariate Analysis

The measured expression levels are analyzed by multivariate analysis. This is a statistical technique for analyzing relationships such as mutual dependency and subordination in a great number of statistical variables. Multivariate analysis basically involves p kinds of variables observed for each of n objects, but there is a variety of versions adapted for effective analysis of such multivariate data. Examples include but are not limited to cluster analysis, principal component analysis and discriminant analysis.

(1) Cluster Analysis

Cluster analysis usually refers to a technique by which, in the field of multivariate analysis, a number of objects for observation (samples) are gathered for similarity (dissimilarity) and classified into groups according to a predetermined basis of calculation (evaluation criterion). That is, cluster analysis merely “classifies” a number of observed samples into similar groups (or dissimilar groups).

Cluster analysis includes hierarchical cluster analysis and non-hierarchical analysis. Hierarchical cluster analysis initially views each sample as a single cluster, combines adjacent clusters and eventually combines the clusters into a single group. On the other hand, in non-hierarchical cluster analysis, the number of clusters to be generated is designated in advance, and hierarchical- cluster analysis is performed on data which are randomly selected from data at certain proportions, using the cluster number as a target. When the target number of clusters is reached, data which were not selected in the previous steps of analysis are combined into the already established clusters in various forms. Hierarchical cluster analysis allows the similarities of samples to be understood visually in the form of a dendrogram, and is often used in the field of biology. Accordingly, it is preferable to use hierarchical cluster analysis in the present invention.

(1-1) Hierarchical Cluster Analysis

In hierarchical cluster analysis, similar samples (clusters) are combined into an upper-hierarchy cluster. As the measure of similarity, the concept of distance is used. For example, supposing there are data {xᵢⱼ} (i=1, 2, . . . , n; j=1, 2, . . . , p) observed for n samples with p kinds of variables, the data {(Xᵢⱼ} is as shown in Table 4:

TABLE 4

| | Variable(j) | | | |
|---|---|---|---|---|
| Sample(i) | 1 | 2 | . . . | p |
| 1 | x₁₁ | x₁₂ | . . . | x₁ₚ |
| 2 | x₂₁ | x₂₂ | . . . | x₂ₚ |
| 3 | x₃₁ | x₃₂ | . . . | x₃ₚ |
| . | . | . | . | . |
| . | . | . | . | . |
| . | . | . | . | . |
| n | xₙ₁ | xₙ₂ | . . . | xₙₚ |

To perform cluster analysis based on the observation data given above, a “distance matrix” is generated, which indicates similarity between samples. The distance is calculated, for example in terms of Euclidian distance, weighted Euclidian distance, normalized Euclidian distance, and Pearson's product moment correlation coefficient.

Euclidian distance is the normally used distance. When an individual Xᵢ is measured by p attributes (variables) and the value of the jth attribute is Xᵢⱼ, Euclidian distance is expressed by the following equation:

$$d ( Xa , Xb ) = \sqrt{{\sum }_{j = 1}^{p} {( Xaj - Xbj )}^{2}} \qquad ( III )$$

Weighted Euclidian distance is expressed by the following equation:

$$d ( Xa , Xb ) = \sqrt{{\sum }_{j = 1}^{p} {kj ( Xaj - Xbj )}^{2}} \qquad ( IV )$$

Weighted Euclidian distance is used when influences on distance are to be varied depending on the attributes. By reducing a weight kj, the contribution of an attribute j to distance is reduced (low data similarity). By increasing the weight, contribution to distance is increased (high data similarity).

Normalized Euclidian distance is expressed by the following equation:

$$d ( Xa , Xb ) = \sqrt{{\sum }_{j = 1}^{p} \frac{{( Xaj - Xbj )}^{2}}{{Sj}_{2}}} \\ {Sj}_{2} = \frac{{\sum }_{i = 1}^{n} {( Xij - \overline{Xmj} )}^{2}}{n - 1} \qquad ( V )$$

wherein \overline{Xmj} is an average from Xlj to Xnj. In this equation, all the attributes are normalized to be variance=1. This equation is used in order to avoid introducing unintended “weights” due to differences in units of measure used for attributes. When calculating distance, since it does not matter where the origin is located, all the attributes are normalized to be average=0 and variance=1, and the Euclidian distance is calculated by using the normalized values.

A distance r (Pierson's product moment correlation coefficient) between case 1 (x₁, x₂, . . . , xᵢ, . . . , xₙ) and case 2 (y₁, y₂, . . . , Yᵢ, . . . , yₙ) is expressed by the following equation:

$$r = \frac{\frac{1}{n - 1} {\sum }_{i = 1}^{n} ( Xi - \overline{X} ) ( Yi - \overline{Y} )}{\sqrt{\frac{1}{n - 1} {\sum }_{i = 1}^{n} {( Xi - \overline{X} )}^{2}} \sqrt{\frac{1}{n - 1} {\sum }_{i = 1}^{n} {( Yi - \overline{Y} )}^{2}}} \qquad ( VI )$$

wherein \overline{X} and \overline{Y} indicate the averages of case 1 and case 2, respectively.

Based on the above concept of distance, the distance between clusters or between a cluster and an individual is calculated and the clusters are merged. Examples of the method of merging are as follows:

Nearest-neighbor method: Of the distances between individuals belonging to different clusters, the minimum value is used as the distance between the clusters. In this method, clusters with shorter distances between the nearest samples are merged as similar clusters.

Furthest-neighbor method: The greatest distance between any two individuals in the different clusters is used as the distance between the clusters. In this method, clusters with shorter distances between the furthest samples are merged as similar clusters.

Centroid method: The distance between barycenters of the respective clusters is used as the distance between the clusters. In this method, clusters whose contained samples having nearby barycenters of samples contained are merged as similar clusters.

Ward method: The sum of the square of Euclidian distances in the clusters is minimized when merging clusters.

Average distance: An average value of all the distances between individuals belonging to each cluster is used as the distance between the clusters.

By any of these classification methods, clusters with a “shortest distance” relationship are assumed to be similar to each other and merged to make an upper-hierarchy cluster. Once clusters in one hierarchy are generated, distances between the generated clusters are again calculated and a distance matrix is generated, and additional upper-hierarchy cluster is generated by calculating for clusters with a minimum distance. In this way, eventually a dendrogram is generated.

In a dendrogram, the samples in a merged cluster at a certain hierarchy have been merged based on a certain similarity relationship. These similar samples can be considered to possess a certain common property, and by analyzing that property it becomes possible to clarify the characteristics of the group of those clusters. For example, when the malignancy is used as an indicator and the samples are viewed in the light of whether they are malignant or not, it is possible to clarify that those cancers belonging to some clusters are malignant and others belonging to other clusters are not.

For example, when, focusing attention on estrogen receptors, certain genes are selected by variance analysis and subjected to cluster analysis, breast cancer samples can be classified into: (i) a group of cases most of which are estrogen receptor-positive; (ii) a group of cases of which most are estrogen receptor-negative; and (iii) a group of cases of which some are estrogen receptor-positive and others are negative. By examining which group a sample to be predicted belongs to, it becomes possible to predict the degree of malignancy, such as whether metastasis or recurrence is likely to occur or not.

Reliability between branches in a dendrogram generated by hierarchical cluster analysis may be calculated by the Bootstrap method, for example. In this method, an empirical probability distribution is assumed that gives a probability of 1/n to each of n samples randomly extracted. Then n random samples are considered (extracted) that allow for overlap from the probability distribution. These randomly re-extracted samples give predicted values which are called bootstrap replicates. The random re-extraction is repeated B times to give B bootstrap replicates, based on which bootstrap estimates of a variance (error) from the original predictions are calculated. The Bootstrap method can be used for evaluating reliability when the normality of probability distribution cannot be assumed or its distribution cannot be fully understood due to complicated statistics. The Bootstrap method is a statistical method well-known to those skilled in the art, and a number of software applications for it are also known. Examples of software useful for the present invention include GeneMaths™ (Applied Maths) and Amos (E-works).

New cancer specimens can be classified based on the classification obtained by cluster analysis, by multivariate analysis such as cluster analysis and discriminant analysis. Examples of the method using cluster analysis include one by which the data of specimens used for the classification and the data of specimens to be predicted are simultaneously subjected to cluster analysis. In another example, the branchings of the dendrogram are traced backwards for classification. When the criteria are simple, classification can be performed by arithmetical computation.

(1-2) Non-hierarchical Cluster Analysis

Examples of non-hierarchical cluster analysis include a method using a self-organizing map (SOM) and the K-means method.

The method using a self-organizing map classifies cancers at individual nodes arranged in k dimensions. The self-organizing map technique is similar to cluster analysis except that all the cancers are re-classified for each operation. The method by the self-organizing map can be used in the two stages of classification of expression patterns and prediction of cancer, as in hierarchical cluster analysis. Further, by performing SOM in combination with the above-mentioned hierarchical cluster analysis, the order of the samples or clusters in a dendrogram can be determined (Chu, S. et al., Science, 282:699, 1998; Tamayo, P., et al., Proc. Natl. Acad. Sci. USA, 96:2907, 1999).

In the K-means method, k initial cluster centroids are appropriately determined, and all of the data are classified into clusters whose centroids they are nearest to. The barycenters of the resulting new clusters are designated as the cluster centers, and classification ends when all of the new cluster centers are identical to the previous ones. The K-means method has a high calculation efficiency and allows the result of cluster analysis to be reached in a short time.

The above-mentioned cluster analysis is a statistical technique well-known to a person skilled in the art. A number of software applications for cluster analysis are also known. Examples of such software useful for the present invention include GeneMaths™ (from Applied Maths), SAS/STAT software (from SAS Institute), and Genesight™ Version 2.0 (from Biodiscovery).

(2) Principal Component Analysis

Principal component analysis is a technique for eliminating correlations between variables from multivariate measurements and for describing the properties of the original measurements by lower-dimensional variables. In the present invention, principal component analysis is employed to eliminate “noise” contained in the gene expression information resulting from a variety of causes and to extract only variations in the gene expression. This enables statistically significant results to be obtained from the gene expression information.

For example, consider a principal component analysis in the case where there are three variables of x, y and w. A principal component is expressed by a linear combination (weighted sum) of the variables, thus: z=ax+by+cw. By substituting values of individual objects for (x, y, w), the principal component values can be obtained. Normally, each variable is normalized to a mean of 0 and a standard deviation of 1. The weight in the linear combination is a correlation coefficient between the variable and the linear component (e.g., a is the correlation coefficient for x and z).

An example of principal component analysis will be described in detail by referring to Table 4. In this example, principal component analysis is performed on n data groups consisting of p kinds of variables. A first principal component score, a second principal component score, and a third principal component score will be calculated.

As a first step of principal component analysis, a first principal component f is determined such that the loss of information possessed by data as a characteristic can be minimized. Specifically, based on the data shown in Table 4, the values of a1, a2, a3, . . . , ap of an eigenvector A=(a1, a2, a3, . . . , ap) of the first principal component f are determined such that the variance of f can be maximized. The values of a1, a2, a3, . . . , ap are calculated such that a1¹+a2²+a3²+ . . . ap²=1. The first principal component scores fl to fn, which indicate the amount of information possessed by individual data, are expressed by the following equations:

$$f1 = a1 \cdot x1l + a2 \cdot x12 + a3 \cdot x13 \\ f2 = a1 \cdot x21 + a2 \cdot x22 + a3 \cdot x23 \\ ⋮ \\ fi = a1 \cdot xil + a2 \cdot xi2 + a3 \cdot xi3 \\ ⋮ \\ fn = a1 \cdot xn1 + a2 \cdot xn2 + a3 \cdot xn3 \qquad ( VII )$$

The more the individual values of fi vary, the more clearly can the characteristics of each data be understood. Therefore, the greatest amount of information can be absorbed by the first principal component f when the variance of f is at a maximum.

Similarly for the second principal component, the values of b1, b2, b3, . . . , bp in an eigenvector B=(b1, b2, b3, . . . , bp) of the second principal component g are calculated such that the loss in the amount of information that cannot be absorbed by the first principal component can be minimized. When the second principal component score for the ith data is gi, gi can be expressed as gi=b1·xi1+b2·xi2+b3·xi3.

Similarly for the third principal component, the values of c1, c2, c3, . . . , and cp in an eigenvector C=(c1, c2, c3, . . . , cp) of the third principal component h are calculated. When the third principal component score for the ith data is hi, hi can be expressed as hi=c1·xi1+c2·xi2+c3·xi3.

Specifically, variance and covariance matrices are obtained from the data in Table 4, and the individual components are calculated from such eigenvalues and eigenvectors that the variance is maximized.

The above-described method of principal component analysis is a statistical technique well-known to a skilled person. A number of software applications for principal component analysis are known. Examples of such software useful for the present invention include GeneMaths™ (from Applied Maths) and SAS/STAT software (from SAS Institute).

(3) Discriminant Analysis

Discriminant analysis is an analysis method for statistically determining, from multivariate data, to which of a number of groups or populations an individual belongs, and analyzing the validity of such discrimination. The discrimination is basically carried out by defining the distance between an individual to be discriminated and each of the groups, and predicting that the individual belongs to the group of the shortest distance. When the number of characteristics to be referred to is one, the statistical distance is determined as:
(Individual measurement−group mean)/(standard deviation of the group) (VIII)

In general, however, Mahalanobis distance, which is extended from the above, is often used.

In the present invention, based on the classification obtained as a result of cluster analysis, a discriminant function for discriminating this classification based on gene expression pattern is created. Using this discriminant function, which group each of the cases to be predicted belongs to is discriminated (determined).

When the variables for multivariate analysis are viewed in terms of the presence or absence of expression of a particular gene or the level of expression, the cases (subjects) can be classified into a group in which a particular gene is expressed at high levels and another group in which the same gene is expressed at low levels. The particular gene may be suitably selected depending on the above-mentioned ratio of total variation to variation within subgroup. By examining to which group a subject specimen belongs based on the result of cluster analysis, it becomes possible, for example, to predict the likelihood whether metastasis or recurrence will occur or not.

4. Prediction of Cancer

The state of cancer is predicted based on the result of multivariate analysis described above. For this purpose, expression patterns characterizing different states of cancer are determined. The states of cancer includes the presence or absence of cancer, and the degree (stage) of progress of cancer. For example, the states of cancer include: (a) whether or not the patient suffers from cancer (presence or absence of cancer); (b) if there is cancer, what degree of its malignancy is (cancer malignancy); (c) whether or not it has metastasized; and (d) what the chances of its recurrence are. Examples of the indices for determining the malignancy include instances of early recurrence, how long the patient has to live, and tumor size.

Multivariate analysis of the above result of gene expression can provide classification results consisting of a group relating to lymph node metastasis and/or early recurrence and a group not relating to either of them. Since lymph node metastasis and recurrence are closely related to prognosis and the malignancy of cancer, they are important factors when predicting prognosis. The frequency of appearance of hormone receptors, lymph node metastasis and recurrence is statistically significantly different for each group. Accordingly, it becomes possible to predict prognosis for new cases by: examining the expression level of the genes having the sequences 1-27 from Table 1, 28-153 from Table 2, and/or 154-289 from Table 3 (preferably, genes having sequences 1-21 from Table 1, sequences 30, 33, 34, 36-42, 44-48, 54-66, 68, 69, 71, 74, 80, 82, 83, 85, 93, 100-103, 112, 115, 116, 118-121 from Table 2, and/or sequences 155, 162, 163, 167-169, 171, 172, 174, 175, 177-180, 188, 190, 193, 198, 211, 222, 242-253, 255-257, 259-261, 263, and 265 from Table 3), and, optionally, other genes which could be considered useful for the classification of cancer, using the method described in the section “1. Quantitative determination of gene expression”; or quantitatively determining the protein products encoded by those genes using the method which will be described in the section “6. Preparation and detection of an antibody,” thereby determining to which of the existing groups of cancer the expression pattern of the specimen belongs.

5. Cancer State Identification System

The identification system of the present invention comprises (a) means of analyzing the expression level of a gene isolated from a test sample; and (b) means of predicting the state of cancer by using the result of analysis as an indicator. The analysis means (a) further comprises a means (also called a detection engine) of detecting the expression level of each of a plurality of genes in a cancer cell or tissue derived from a primary cancerous lesion and in a normal tissue, and a means (also called an analysis engine) of analyzing the resultant detection values.

(1) Gene Expression Detection Engine

In the present invention, the detection data obtained as described above may be converted into digital information and used for the detection of gene expression.

(2) Analysis Engine

The analysis engine is a means of performing multivariate analysis, for example cluster analysis, based on the data (gene expression level) provided by the detection engine. This analysis process can classify the genes into a group of genes with high expression levels and a group of genes with low expression levels. Further, this means can classify the samples into estrogen-receptor expression positive, negative and positive/negative mixed groups, for example.

FIG. 3 shows the block diagram of an example of the prediction system according to the invention.

The prediction system of FIG. 3 comprises a CPU 301, a ROM 302, a RAM 303, an input unit 304, a transmitter/receiver unit 305, an output unit 306, a hard disc drive (HDD) 307, and a CD-ROM drive 308.

CPU 301 controls the cancer state prediction system as a whole according to a program stored in ROM 302, RAM 303 or HDD 307, and executes a prediction process to be described later. ROM 302 stores the program, such as for commanding performing processes necessary for the operation of the prediction system. RAM 303 temporarily stores data necessary for executing the prediction process. The input unit 304 includes a keyboard and/or a mouse, for example, which is operated when inputting necessary conditions for executing the prediction process. The transmitter/receiver unit 305 executes a data transmission/reception process with a database 310, for example, via a communication line based on the commands from CPU 301. The output unit 306 displays various conditions or expressed gene detection data inputted via the input unit 304, according to commands from CPU 301. Examples of the output unit 306 include a computer display and a printer. HDD 307 stores the expression pattern information about various kinds of genes in a cell or tissue. It reads the stored program, data or the like in response to commands from CPU 301, and stores it in RAM 303, for example. Based on commands from CPU 301, the CD-ROM drive 308 reads a program, data or the like from a prediction program stored in a CD-ROM 309, and stores it in RAM 303, for example.

CPU 301 supplies the data received from the input unit, for example, to the output unit 306, while executing the prediction for the likelihood of metastasis or recurrence of cancer on the basis of the data received from the stored database. The database refers to the storage of information relating to the level of gene expression obtained as described above (including both an absolute level and a relative level).

FIGS. 4 and 5 shows the flowchart of an example of prediction of the states of cancer executed by the program shown in FIG. 3 in the case where gene expression patterns were analyzed.

Referring to FIG. 4, a cluster analysis device 401 will be hereafter described as an example of the multivariate analysis device. The cluster analysis device 401 generates clusters for the above prediction process. First, gene expression data is fed via an external database search/input means 402. The external database search/input means 402 has a function to access the existing, various kinds of external databases, preferably by using a predetermined keyword, in order to collect sample data to be subjected to multivariate analysis (such as cluster analysis). The above data input operation is repeated until data input is finalized. The information obtained from individual tissues or cells is stored in a sample data storage means 403 by the input of data, subjected to cluster analysis, or registered in the database.

The sample data stored in the sample data storage means 403 is inputted to a data optimization means 404, where the data is optimized for multivariate analysis. Examples of data optimization include standardization by a median, standardization by a z-score, setting of a maximum and minimum value, and logarithmic transformation, of which the one most suitable for the samples used can be selected.

A variable list output means 405 displays a list of the variables of the sample data to be analyzed for example, by cluster analysis.

Next, the user selects variables from the variables displayed on the list by the variable list output means 405, using the function of a variable selection means 406.

The selection of the variables using the variable list output means 405 is carried out such that the user can freely select one or more particular variables. Typically, since there are a number of candidates for variables, the user should be able to select any of those candidates.

As the user selects particular variables, this information is inputted to an evaluation sample data file generating means 407, together with the sample data. The evaluation sample data file generating means 407 generates a data file for the evaluation samples.

The data file for the clusters for evaluation is then transmitted to an evaluation means 408, where the degree of cluster separation is evaluated. The evaluation formula for the evaluation of the degree of cluster separation can be defined in various ways.

The result of the evaluation of cluster separation degree by the evaluation means 408 is given to a cluster classification means 409. The cluster classification means 409 receives the evaluation result from the evaluation means 408, refers to evaluation conditions set in an evaluation condition setting means 412, determines an optimum cluster classification, and, in the case where a cluster classification continuation/termination condition is set, determines whether cluster classification should be continued or terminated. In the case where the cluster classification continuation/termination condition is not set, the cluster classification means 409 lets the user decide whether cluster classification should be continued or terminated. If the user chooses to continue with cluster classification, the cluster classification means 409 outputs the optimum cluster classification obtained in the most recent procedure, and a signal for the continuation of cluster classification. The signal for the continuation of cluster classification later constitutes a command for bringing the procedure back to the process in the variable list output means 405 after the process in a dendrogram editing means 411.

On the other hand, if the cluster classification means 409 has decided to discontinue the cluster classification operation, cluster classifications that are optimal at that point in time are identified, and a signal for the discontinuation of the cluster classifying operation is output. This signal for the discontinuation of the cluster classifying operation later constitutes a command for terminating the cluster analysis process after the process in the dendrogram editing means 411 is performed.

After the process in the cluster classification means 409 is completed, the process in a dendrogram generating means 410 is initiated. The dendrogram generating means 410 receives the cluster classification determined by the cluster classification means 409, and displays a dendrogram based on the cluster classification and the attributes of the variables relating to individual cluster classifications. The cluster classification dendrogram thus generated by the dendrogram generating means 410 allows the user to visually grasp the current state of cluster classification. Together with the generation of the dendrogram, the dendrogram generating means 410 also displays colored, patterned or otherwise decorated cells to allow the user to visually grasp the gene expression levels on whose basis the dendrogram was generated. Next, the dendrogram editing means 411 lets the user edit the cluster classification dendrogram generated by the dendrogram generating means 410 by addition, modification or deletion of the cluster classification on the screen of the display device. The addition, modification or deletion of the cluster classification is carried out by the user: designating a particular cluster and further designating the variable of a cluster which is to be classified lower than that particular cluster; merging a plurality of clusters; or deleting the branch of a certain cluster classification, for example, using a processing instruction input device on the screen. The dendrogram editing means 411 provides a variety of tools for assisting the user's editing operation on the screen. The dendrogram editing means 411 reads the significance of each revision of the cluster classification by the user and automatically modifies the data file for each cluster according to that significance. Preferably, the dendrogram editing means 411 asks the user whether the cluster classification by the cluster classification means 409 should be continued or terminated and lets the user input a final decision.

As a result, if the repetition of cluster classification is to be continued, the process is returned to the variable list output means 405, and the processes from the variable list output means 405 to the dendrogram editing means 411 are repeated.

Based on the thus analyzed data, the state of cancer such as the possibility of metastasis or recurrence can be determined by examining to which cluster the cancer specimen to be tested has been classified.

FIG. 5 shows an device for predicting the result of cluster analysis. A prediction device 501 constitutes a processing means by which a data file and an evaluation condition that is set via a cluster 513 outputted by the cluster analysis device of FIG. 4 can be integrated in an evaluation means 508, the data file being obtained via an external database search/input means 502, a sample data storage means 503, a data optimizing means 504, a variable list output means 505, a variable selection means 506 and an evaluation sample data file generating means 507. The individual means from the external database input means 502 to the evaluation sample data file generating means 507 perform the same processes as those in the cluster analysis device shown in FIG. 4. In the case where a prediction process is to be performed on the basis of the clusters which are the output of FIG. 4, a cluster 513 is inputted to an evaluation condition setting means 512. Then, an evaluation means 508, a prediction means 509, a prediction result generating means 510 and a prediction result editing means 511 perform their individual processes. When certain sample data is to be subjected to the prediction process together with the clusters which have been obtained as output of FIG. 4, the sample data is processed from the external database search/input means 502 to the evaluation sample data file generating means 507, and then integrated in the evaluation means 508 with the cluster data from the evaluation condition setting means 512.

After the process in the prediction means 509 is completed, the prediction result generating means 510 starts its process. The prediction result generating means 510 receives the prediction result produced in the prediction means 509 and displays a chart (figure) based on that prediction result and the attributes of the variables relating to the individual cluster classifications. Based on the prediction result chart generated by the prediction result generating means 510, the user can visually grasp the predicted state. In addition to the prediction result chart, the prediction result generating means 510 displays the levels of gene expression on which the chart was based, by means of letters and/or colored or patterned cells, so that the user can visually grasp the gene expression levels. Thereafter, the prediction result editing means 511 lets the user edit the prediction result chart generated by the prediction result generating means 510, by way of addition, modification and/or deletion of the cluster classifications on the screen of the display device. The prediction result editing means 511 provides a variety of tools assisting the user's editing operations on the screen. The prediction result editing means 511 reads the significance of each revision of the prediction result by the user and automatically modifies the data file of each prediction result according to that significance. Preferably, the prediction result editing means 511 asks the user to select whether the prediction operation by the prediction means 509 should be continued or terminated, so that the user can input his or her final decision.

If a repetition process for prediction is to be continued, the procedure returns to the variable list output means 505, and the above-described processes from the variable list output means 505 to the prediction result editing means 511 are repeated.

For example, when expression levels for 10 or more genes in 100 to 500 cases are measured, these data are stored as population data and cluster analysis is performed on the data for the genes to be analyzed, together with the parent (population) data, so that the genes to be analyzed can be classified into one or another group. If a particular classified group has a low probability of cancer metastasis or recurrence, it can be predicted that it is unlikely that the cancer in the individual as a subject of the cluster analysis will metastasize or recur.

The present invention provides not only the program for the means for predicting the metastasis or recurrence of cancer, but also a recording medium in which that program is recorded. The recording medium may be computer-readable. Examples of the medium include a floppy disc (FD), a magneto-optical disc (MO), a CD-ROM, a hard disc, a ROM and a RAM.

6. Production of Antibody and Detection

In the present invention, in order to measure the level of gene expression, a protein product encoded by that gene can be quantitatively determined. The protein product can be immunologically quantitatively determined by using an antibody against the protein. Hereafter, the method of production of the antibody and its quantitative determination will be described.

(1) Expression and Purification of a Protein

(i) Production of an Expression Vector

A recombinant vector for expression of a protein can be obtained by linking the above-mentioned gene to an appropriate vector. A transformant can be obtained by introducing the recombinant vector into a host so that the target gene can be expressed.

As the vector, a phage or plasmid that is capable of autonomously growing in a host microorganism is used. Examples of a plasmid DNA include those derived from Escherichia coli, Bacillus subtilis and yeast. An example of a phage DNA is lambda phage. Further, animal viruses such as retrovirus and vaccinia virus, and insect virus vectors such as baculovirus can be used.

In order to insert the gene according to the invention into the vector, a method is adopted, for example, whereby purified DNA is cleaved by an appropriate restriction enzymes and inserted into a restriction enzyme site or a multi-cloning site of an appropriate vector DNA to ligate to the vector.

For ligating the DNA fragment to the vector fragment, a known DNA ligase is used. The DNA fragment and the vector fragment are annealed and ligated, thereby producing a recombinant vector.

The host to be used for transformation is not particularly limited as long as it allows the target gene to be expressed therein. Examples of the host include bacteria (such as E. coli. and Bacillus subtilis), yeast, animal cells (such as COS cells and CHO cells), and insect cells.

The gene can be introduced into the host by a known method (such as a method using calcium ions, electroporation, a spheroplast method, a lithium acetate method, a calcium phosphate method, lipofection, etc.).

(ii) Preparation of a Protein

In the present invention, the protein which is expressed by the above gene can be obtained from a culture of the above transformant possessing the target gene. The “cultured product” refers to any of (a) culture supernatant, or (b) a cultured cell or cultured microorganism, or homogenate thereof. The transformant of the invention is cultured in a culture medium by a usual method of cultivating a host. Culturing is typically performed by shaking culture or aeration culture with stirring. During culturing, antibiotics such as ampicillin or tetracycline may be added to the medium as needed.

After culturing, in the case where the intended protein is produced in the microorganism or cell, the protein is extracted by homogenizing the microorganism or cell. In the case where the intended protein is secreted from the microorganism or cell, the culture medium is used as is, or the microorganism or cell is removed by centrifugation, for example. Thereafter, the intended protein can be isolated from the culture and purified by a conventional biochemical method for the isolation and purification of proteins, such as ammonium sulfate precipitation, gel chromatography, ion-exchange chromatography, affinity chromatography, either individually or in combination. Whether the intended protein have been obtained or not can be confirmed by SDS polyacrylamide gel electrophoresis, for example.

In the present invention, not only the entire purified protein but also its partial fragments can be used. The term “partial fragments” is used herein for fragments regardless of their length as long as they contain amino acid residues selected from the amino-acid sequences of proteins encoded by the genes 1-289 from Tables 1-3 or, in some cases, the other genes having equivalent functions to the above genes.

The partial fragments can be prepared in the form of peptide fragments by conventional peptide synthesis, for example. Peptides may be chemically synthesized in a conventional manner. Such conventional synthesis includes an azide method, an acid chloride method, an acid anhydride method, a mixed acid anhydride method, a DCC method, an activated ester method, a carboimidazole method, and an oxidation-reduction method. The synthesis may be performed by either a solid-phase or liquid-phase method. Further, in the present invention, the synthesis may be performed by a commercially available automatic peptide synthesizer (such as the automatic peptide synthesizer PSSM-8) from SHIMADZU Corporation).

(2) Preparation of an Antibody

The term “antibody” herein refers to an antibody molecule as a whole or its fragments (such as Fab or F(ab′)₂ fragments) which can bind to the above-mentioned protein or its partial fragments as the antigen. The antibody may be either a polyclonal antibody or a monoclonal antibody. In the present invention, the antibody (polyclonal or monoclonal antibody) can be generated by e.g. the following method.

(i) Monoclonal Antibody

The prepared protein or its fragments is administered as an antigen to a mammal, such as a rat, mouse, or rabbit. An adjuvant such as Freund's complete adjuvant (FCA) or Freund's incomplete adjuvant (FIA) may be used as needed. The immunization is performed mainly by intravenous, subcutaneous, or intraperitoneal injection. The interval of immunization is not particularly limited and immunized one to ten times at the intervals of several days to weeks. Antibody-producing cells are collected one to 60 days after the last day of immunization. Examples of the antibody-producing cell include a pancreatic cell, a lymph node cell, and a peripheral blood cell.

To obtain a hybridoma, an antibody-producing cell and a myeloma cell are fused. As the myeloma cell to be fused with the antibody-producing cell, a generally available established cell line can be used. Preferably, the cell line used should have a drug selectivity and properties such that it cannot survive in a HAT selective medium (containing hypoxanthine, aminopterin, and thymidine) in unfused form and can survive only when fused with an antibody-producing cell. The myeloma cell may include, for example, mouse myeloma cell lines such as P3X63-Ag. 8. U1(P3U1) and NS-1.

Next, the myeloma cell and the antibody-producing cell are fused. For the fusion, the cells are mixed (preferably at the antibody-producing cell to myeloma cell ratio of 5:1) in a culture medium for animal cell which does not contain serum, such as DMEM and RPMI-1640 media, and fused in the presence of a cell fusion-promoting agent (such as polyethylene glycol). The cell fusion may also be carried out by using a commercially available cell fusion device using electroporation.

The desired hybridoma is selected from the post-fusion cells. For example, a cell suspension is appropriately diluted in e.g. the RPMI-1640 medium containing fetal bovine serum and then plated on a microtiter plate. A selection medium is added to each well, and the cells are cultured with appropriately replacing the selection medium. As a result, the cells which grow about 14 days after the start of culturing in the selection medium can be obtained as the hybridoma.

The culture supernatant of the grown hybridoma is then screened for the presence of an antibody which reacts with the intended protein. This can be carried out in a conventional manner, such as by enzyme immunoassay or radioimmunoassay, for example. The fused cells are cloned by the limiting dilution to establish a hybridoma which produces the desired monoclonal antibody.

Examples of the method of collecting the monoclonal antibody from the established hybridoma include the conventional cell culture method and ascites production method.

If it is necessary to purify the antibody in the above-described antibody collecting method, a known method such as ammonium sulfate precipitation, ion exchange chromatography, gel filtration, or affinity chromatography, or a combination thereof, may be used.

(ii) Production of Polyclonal Antibody

In order to prepare a polyclonal antibody, immunization step is conducted in an animal in the same manner as described above. After 6 to 60 days from the last day of immunization, antibody titer is measured by enzyme-linked immunosorbent assay (ELISA), enzyme immunoassay (EIA), or radioimmuno assay (RIA), for example. Blood is collected on the day when the maximum antibody titer was measured, to obtain an antiserum. Thereafter, the reactivity of the polyclonal antibody in the antiserum is measured by ELISA, for example.

(3) Detection

The protein can be detected by a known technique such as Western blotting, RIA or ELISA. A commercially available kit may also be used for detecting the protein.

7. Drug Design Based on the Result of the Method According to the Invention

Systems are being designed for designing compounds which specifically inactivate an active site of a target molecule related to the development of a disease, or screening compounds for recovering the function of an inactivated protein by changing its conformation. If the underlying differences in the mechanism causing the diseases with the same diagnosis or similar symptoms could be clarified at the molecular level, treatment can be tailored to individual needs (“Personalized medicine”) by, for example, using different drugs for different mechanisms.

It is known that the state of cancer (such as malignancy) is determined not only by the gene causing the cancer but also other genes. The expression of those genes varies from person to person. In the present invention, the gene expression patterns are influenced by genes that are un-related to cancer, as well as by the cancer-causing gene. The present invention takes advantage of the result of expression of genes exhibiting such a relationship with the state of cancer to target certain of those genes and design a drug useful for cancer treatment, in order to reduce cancer malignancy and treat cancer. Specifically, the gene expression in a cancer specimen whose state of cancer (such as the presence or absence of cancer, malignancy, presence or absence of metastasis or recurrence) is predicted to be high-risk according to the method of the invention can be regulated such that the specimen has an expression pattern which is predicted to be low-risk. For example, the invention enables a drug to be designed which can suppress or enhance gene expression such that an expression pattern characteristic of high grade of malignancy can be turned into an expression pattern characteristic of low grade of malignancy. “High-risk” herein refers to a state where there is at least one of the following states: a state where the pathological malignancy of the cancer is high; a state where metastasis occurs at more than one place; a state where more than one kinds of cancer are present; and a state where there is a recurrence of cancer within 36 months of healing. “Low-risk” herein refers to a state where the pathological malignancy of the cancer is not high, the state where there is no metastasis, or the state where the cancer does not recur for more than five years. These states are only exemplary and may be changed as the treatment methods are improved.

The invention can therefore reduce the likelihood of a metastasis or recurrence of cancer and reduce the malignancy. It can also provide effective preventive treatment (including prevention for metastasis and recurrence) or therapeutic treatment against high-malignancy cancers.

First, target genes whose expression is to be regulated are selected. Based on the result of gene expression patterns cancer specimens whose malignancy is predicted to be high according to the method of the invention, the genes are classified into a group of genes with high expression patterns and another group of genes with low expression patterns, and each of the thus classified genes is used as a target. More than one target gene can be selected. A plurality of genes used for cluster analysis may also be used as targets.

After determining the target genes, a drug is designed which can regulate the expression of the genes or the activity of the gene products. “Regulate the expression of the genes or the activity of gene products” herein refers to blocking, reducing, enhancing and/or facilitating the expression of the genes or the activity of the gene products.

In the case where the expression of a gene is to be suppressed, a drug is designed by which the expression of the gene can be directly suppressed. An example of a conventional method is the antisense method. Alternatively, a drug may be designed by which the function of a gene expression product (protein) can be suppressed. In this case, an antibody against the protein may be used. Further, an inhibitor of the protein activity may also be used.

The antisense method involves having an antisense sequence specifically bind to the sequence of the target gene and suppressing the expression of a target gene. Preferably, the expression of a gene that expresses at high levels is suppressed. “Expresses at high levels” means that the intracellular level of mRNA is higher than average values. An antisense sequence is a nucleic acid sequence that can specifically hybridize to at least a part of a target sequence. The antisense sequence binds to the cellular mRNA or genome DNA and blocks its translation or transcription, thereby blocking the expression of the target gene. For the antisense sequence, any nucleic acid substance may be used as long as it can block the translation or transcription of the target gene. For example, such nucleic acid substance includes DNA, RNA and any desired nucleic acid mimetics. Thus, genes expressed in a cancer specimen with high malignancy are selected from the genes having the nucleotide sequences 1-289 from Tables 1-3 and/or, in some cases, other genes having similar functions, and an antisense nucleic acid (oligonucleotide) sequence is designed such that it is complementary to a part of the sequence. Examples of the target genes whose expression is to be suppressed in the present invention include the genes having sequences 4 and 7 from Table 1; sequences 28, 29, 31, 32, 35, 43, 49-53, 67, 70, 72, 73, 75-79, 81, 84, 86-92, 94-99, 104-111, 113, 114, 117, and 122-153 from Table 2; and sequences 155, 162, 163, 167-169, 171, 172, 174, 175, 177-180, 188, 190, 193, 198, 211, 222, 242-253, 255-257, 259-261, 263 and 265 from Table 3. Preferably, one or more of these genes are used.

The length of the antisense nucleotide acid sequence to be designed is not particularly limited as long as it can suppress the expression of the target gene. . The length may be, for example, 10-50 bases long or, preferably, 15-25 bases long. Oligonucleotides can be readily chemically synthesized by known methods.

The antisense sequence can be delivered to a target location (such as a cancer cell) by a variety of known administration methods employing an expression vector. Examples of the administration methods include a method using a recombinant expression vector such as a chimera virus or a colloidal dispersion system, and a method employing a variety of viral vectors including a retrovirus vector and adeno-associated virus vector.

Molecular analogs of an antisense oligonucleotide may also be used for the purposes of the present invention. A molecular analog has high stability and distribution specificity, for example. An example of the molecular analog is an antisense oligonucleotide linked to a chemically reactive group, such as iron-binding ethylenediaminetetraacetic acid.

Examples of the vector that can be used for antisense gene therapy include, but are not limited to, adenovirus, herpesvirus, vaccinia virus, and RNA viruses such as asretrovirus.

Other examples of the gene delivery system that can be used for administering the antisense sequence to the target tissue or cell include a colloidal dispersion system, a liposome-induced system, and an artificial virus envelope. Specifically, a macromolecular complex, a nano-capsule, a microsphere, beads, oil-in-water type emulsion, micelle, mixed micelle, and liposome, for example, may be used as a delivery system.

According to the drug design of the invention, an antisense oligonucleotide that can bind (preferably specifically bind) to the sequence of the target gene determined on the basis of the result obtained by the cancer prediction method of the invention is administered in a therapeutically effective amount in order to block the translation of the mRNA from the gene. For example, the antisense oligonucleotide may be administered systemically such as intravenously or intraarterially, as normally done; or it may be administered locally into the cancer tissue. Optionally, any of these administration modes may be used in combination with catheter techniques and surgical techniques, for example.

The dosage of antisense oligonucleotide administered may vary depending on age, sex, symptoms, administration routes, administration frequency, and dosage form. However, a conventional method in the relevant art may be appropriately selected and used.

When an antibody is used, it can be either polyclonal or monoclonal. Further, antibody fragments may be used. An antibody can be prepared by the method described above in the section “5. Preparation of an antibody and detection.”

The dosage of antibody administered may vary depending on age, sex, symptoms, administering routes, administration frequency, and dosage form. However, it may be appropriately determined by a conventional method in the relevant art.

When the antibody is administered (parenterally), various routes of administration may be selected, such as intravenous injection (including continuous infusion), intramuscular injection, intraperitoneal injection, subcutaneous injection, and suppository. In the case of a preparation for injection, the antibody is supplied in the form of a unit-dosage ampule or a multi-dosage container.

On the other hand, if the purpose is to enhance the expression of a gene, a drug is designed by which the expression of the gene can be directly enhanced. A conventional method uses a vector (targeting vector) in which the target gene is inserted. A targeting vector refers to the nucleic acid sequence of an expressed gene connected to the promoter sequence. Preferably, the vector is used such that a low-expression gene is expressed. “Low-expression” refers to the intracellular level of mRNA being lower than average values.

One method for enhancing the gene expression is to connect a strong expression regulatory sequence (promoter) to the sequence of the target gene to thereby enhance the expression of the target gene. First, a promoter which can be active in a host cell is operably liked to upstream of the target gene. By inserting this into a vector such as a viral vector, a targeting vector can be constructed which can express the target gene in the host cell at high levels. “Operably liked” herein means to link the promoter and the target gene together such that the target gene can be expressed under the control of the promoter in the host cell into which the target gene is introduced. As a result, the expression of the target gene is enhanced by the strong action of the promoter. Accordingly, a gene which is expressed at low levels in a high-malignancy cancer specimen is selected from the genes having nucleotide sequences 1-289 from Tables 1-3 and/or, in some cases, from other genes having similar functions, and a strong promoter is linked to that gene. In the present invention, examples of the target gene for expression enhancement include the genes having sequences 1-3, 5, 6, 8-19 and 21 from Table 1, sequences 30, 33, 34, 36-42, 44-48, 54-66, 68, 69, 71, 74, 80, 82, 83, 85, 93, 100-103, 112, 115, 116 and 118-121 from Table 2, and sequences 154, 156-161, 164-166, 170, 173, 176, 181-187, 189, 191, 192, 194-197, 199-210, 212-221, 223-241, 254, 258, 262, 264 and 266-289 from Table 3. Preferably, one or more of those genes are used.

Examples of the strong promoter which can be active in the host cell, for example when the host is aa animal cell, include, but are not limited to, a Rous sarcoma virus (RSV) promoter, a cytomegalovirus (CMV) promoter, an early or late promoter of simian virus (SV40), a mouse mammary tumor virus (MMTV) promoter, and a CAG promoter.

The vector into which the target gene and the promoter are inserted is a vector that can be compatible to the host cell, such as one which contains genetic information that can be replicated in the host cell and thus multiply autonomously, and which can be isolated from the host cell for purification and has a detectable marker. Accordingly, for example a cis-element such as an enhancer, a splicing signal, a poly-A addition signal, a selection marker, or a ribosome binding sequence (SD sequence), as well as a target gene and a promoter, can be connected to the vector as needed. Examples of the selection marker include a dihydrofolate reductase gene, an ampicillin-resistant gene, and a neomycin-resistant gene. Examples of the vector include, but are not limited to, in the case where a mammalian cell is used as the host cell: plasmids such as pRC/RSV and pRC/CMV (from Invitrogen); vectors containing a virus-derived autonomously replicating origin, such as bovine papilloma virus plasmid pBPV (from Amersham Pharmacia) and EB virus plasmid pCEP4 (from Invitrogen); and virus vectors such as vaccinia virus, retrovirus and adenovirus.

In the case where a vector which previously possesses a promoter being active in the host cell is used, the target gene may be inserted downstream of the promoter such that the vector-possessing promoter is operably linked to the target gene. For example, the above-mentioned plasmids pRC/RSV, pRC/CMV or the like have a cloning site downstream of the promoter which is active in an animal cell. Thus, by inserting the target gene into the cloning site and thus introducing it to the animal cell, the target gene can be expressed.

In order to insert the target gene and promoter into the vector, a method is employed by which, for example, a purified DNA is inserted into the restriction enzyme site or multicloning site of an appropriate vector DNA.

The thus prepared targeting vector may be directly administered to the patient (in vivo method). Alternatively, it may be introduced into a cell obtained from the patient, preferably a stem cell, and a cell in which the target gene is to be expressed is selected and then the cell may be administered to the patient (ex vivo method). The targeting vector may be directly administered by intravenous injection (including continuous infusion), intramuscular injection, intraperitoneal injection, and subcutaneous injection, or via other route of administration. The introduction of the targeting vector into the cell may be carried out by a conventional gene-introducing method such as, for example, a calcium phosphate method, a DEAE dextran method, electroporation, or lipofection. The selection of the cell which expresses the target gene may be carried out by utilizing a selection marker, as known in the art. The administration of the cell in which the target gene is expressed may also be carried out in the same manner as in the case of the direct administering of the targeting vector.

In another example of the drug design according to the present invention, a targeting vector into which the sequence of a target gene determined on the basis of the result of the cancer prediction method of the invention and a promoter bound to the target gene are inserted is administered in a therapeutically effective amount either directly or via a cell into which the vector has been introduced, in order to enhance the expression of the gene.

The dosage of the targeting vector administered varies depending on age, sex, symptoms, administration routes, administration frequency, and dosage form, but it may be appropriately determined by a conventional method in the art.

Alternatively, an expression product of the target gene may be directly administered. In this case, a great amount of expression products can be obtained by using a conventional recombinant protein production method. For example, the expression products of the target gene can be produced by using Escherichia coli, for example. The expression products of the target gene may be administered in the same manner as the targeting vector. The dosage of the expression products administered varies depending on age, sex, symptoms, administration routes, administration frequency, and dosage form. However, it may be appropriately determined by a conventional method in the art.

Various types of preparations may be formulated in a conventional manner by appropriately selecting pharmaceutically acceptable substances that are typically used for the formulation of preparations, such as excipient, disintegrant, lubricant, surfactant, dispersing agent, buffering agent, preservative, solubilizer, antiseptic agent, stabilizing agent, and isotonizing agent.

### BRIEF DESCRIPTION OF THE DRAWINGS

FIG. 1 shows an outline of the cancer prediction method according to the present invention.

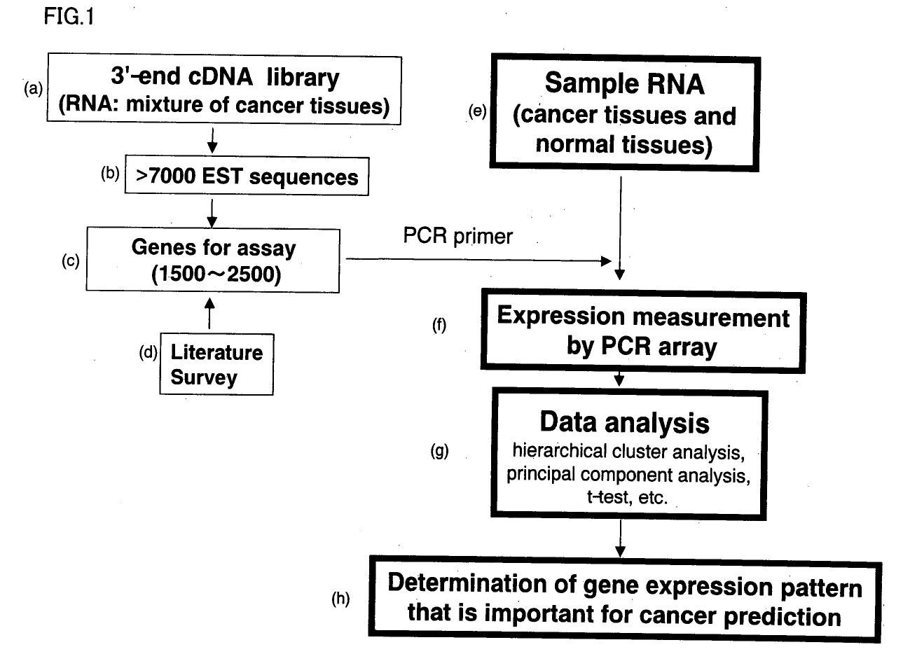

FIG. 2 shows a scheme of adaptor-tagged competitive PCR.

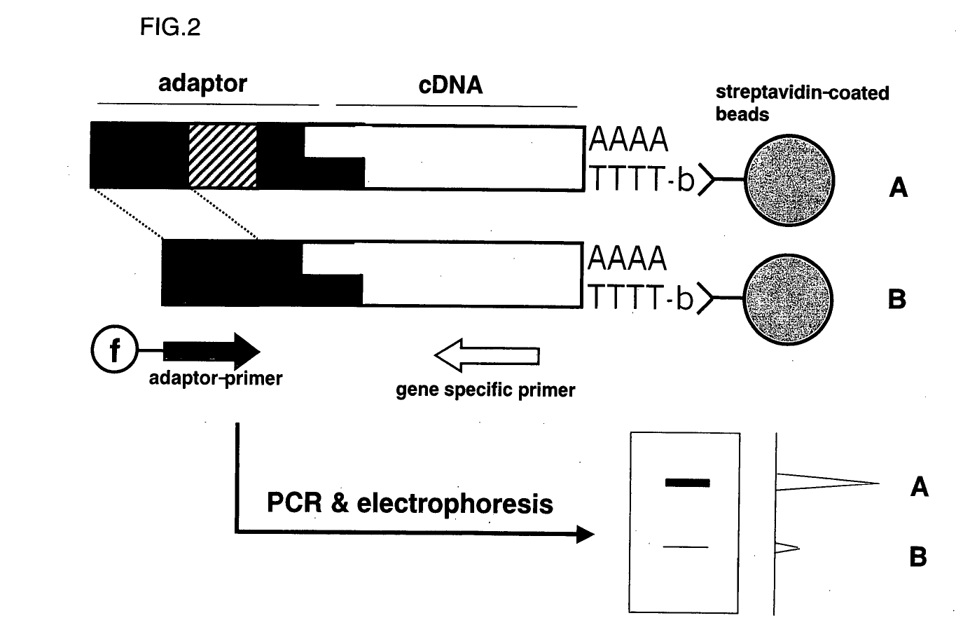

FIG. 3 shows a block diagram of a metastasis or recurrence identification system.

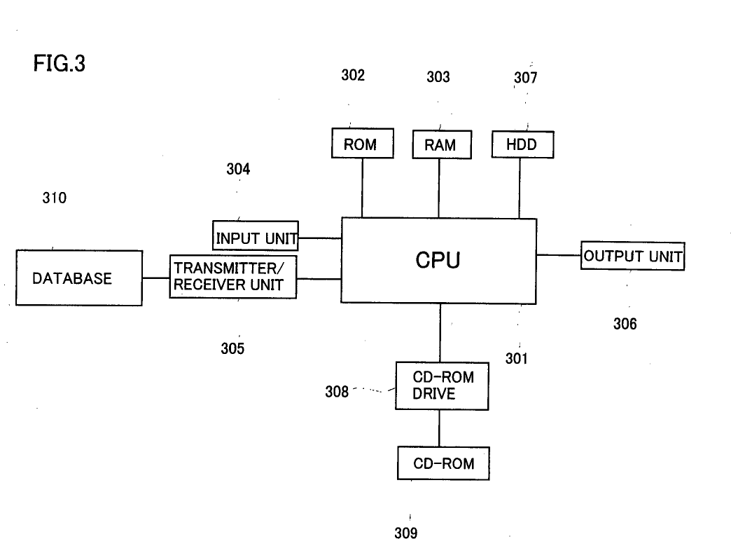

FIG. 4 shows a flowchart of the processes performed by a metastasis or recurrence identification program.

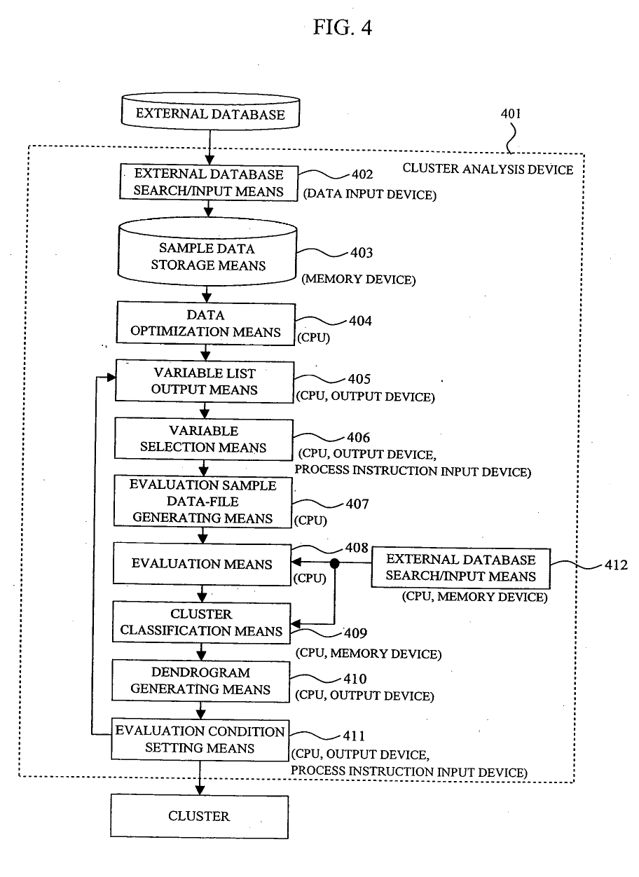

FIG. 5 shows another flowchart of the processes performed by a metastasis or recurrence identification program.

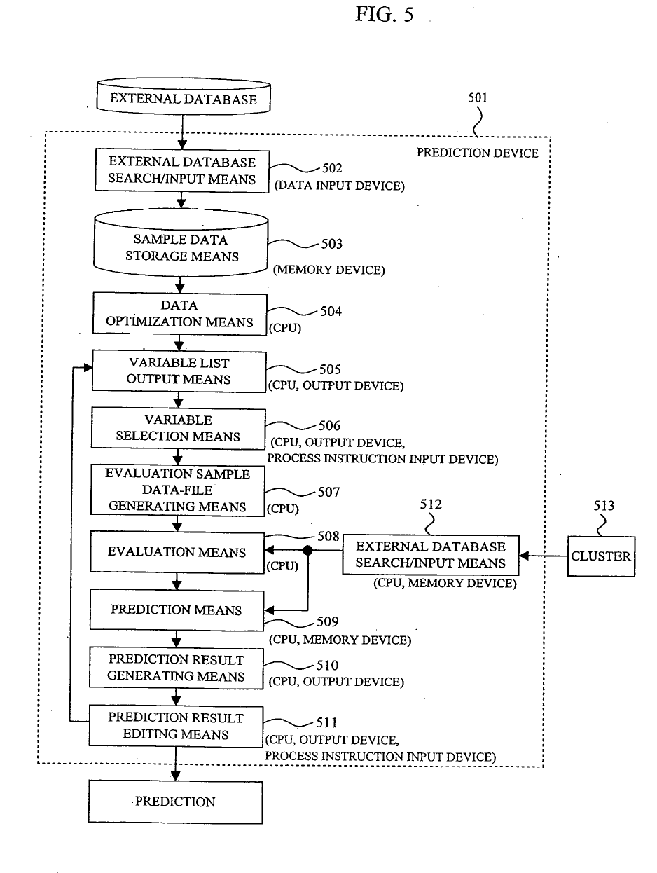

FIG. 6 shows the results of cluster analysis of genes from 179 cases for breast cancer.

FIG. 7 shows the results of cluster analysis of genes from 301 cases of breast cancer.

FIG. 8 shows the results of cluster analysis of genes from 115 cases for colon cancer.

FIG. 9 shows the results of cluster analysis of genes belonging to a M cluster.

FIG. 10 shows the results of cluster analysis of genes belonging to a P cluster.

FIG. 11 shows the results of principal component analysis of an M cluster.

FIG. 12 shows the results of principal component analysis of a P cluster.

FIG. 13 shows the results of principal component analysis of the M and P clusters.

### DESCRIPTION OF REFERENCE NUMERALS

301: CPU, 302: ROM, 303: RAM, 304: input unit, 305: transmitter/receiver unit, 306: output unit, 307: HDD, 308: CD-ROM drive, 309: CD-ROM, 310: database,

401: cluster analysis device, 402: external database search/input means, 403: sample data storage means, 403: sample data storage means, 404: data optimization means, 405: variable list output means, 406: variable selection means, 407: evaluation sample data file generating means, 408: evaluation means, 409: cluster classification means, 410: dendrogram generating means, 411: dendrogram editing means, 412: evaluation condition setting means,

501: prediction device, 502: external database search/input means, 503: sample data storage means, 504: data optimization means, 505: variable list output means, 506: variable selection means, 507: evaluation sample data-file generating means, 508: evaluation means, 509: prediction means, 510: prediction result generating means, 511, prediction result editing means, 512: evaluation condition setting means, 513: cluster

### BEST MODES OF CARRYING OUT THE INVENTION

Hereafter the present invention will be further described in detail by way of examples. It should be noted that the technical scope of the invention is not limited by these examples.

### (EXAMPLE 1)

### Adaptor-Tagged Competitive PCR Utilizing a Breast Cancer Specimen

The expression levels of 2412 genes were measured in 110 cases (98 cases of breast cancer, one case of male breast cancer, one case of thyroid cancer, and 10 cases of normal tissue) by an adaptor-tagged competitive PCR method.

Specifically, the tissues were homogenized and a total RNA was obtained from the above cancer or tissue by a guanidine isothiocyanate method. Then, a chemically synthesized biotinylated oligo (dT)18 primer was added to 7 μL of distilled water containing the total RNA (3 μg). The mixture was heated at 70° C. for 2 to 3 minutes, and was further maintained at 37° C. for one hour to synthesize cDNA. To the resultant single-stranded cDNA was added a reaction solution containing a DNA synthase, and they were reacted at 16° C. for one hour and then at room temperature for one hour, to synthesize double-stranded cDNA.

When the reaction had been completed, 3 μL of 0.25M EDTA (pH7.5) and 2 μL of SM NaCl were added, and phenol extraction process and ethanol precipitation process were carried out. The resultant cDNA was dissolved in 120 μL of distilled water. When the cleaving reaction with the restriction enzymes had been completed, the reaction solution was heated at 75° C. for 10 minutes, diluted with 9 volumes of distilled water and then used for a reaction for adding an adaptor, as described below.

A PCR reaction was conducted by using a gene specific primer and an adaptor primer. Each solution of the above composition was subjected to 30-35 cycles of reaction, each cycle consisting of heating at 94° C. for 30 seconds, at 55° C. for one minute, and at 72° C. for one minute. Thereafter, the solution was reacted at 72° C. for 20 minutes. When the reaction had been completed, the solution was maintained at 37° C. for one hour.

The final product was thermally denatured and then 0.5 μL of it was analyzed by ABI 3700 DNA Analyzer to determine the expression levels of each genes.

### (EXAMPLE 2)

### Cluster Analysis for Breast Cancer

As a group of genes useful for classification, genes satisfying the following equation were selected:
(variance in cancer specimens)/(variance in normal specimens)≧1.20
As a result, 152 genes satisfying the above equation were selected. From those 152 genes, 21 were further isolated (selected), based on differences in expression levels between estrogen receptor-positive and negative groups (p<3.85×10⁻⁵). Table 1 shows a list of the isolated genes, in which sequences 1-21 are those of the isolated genes.

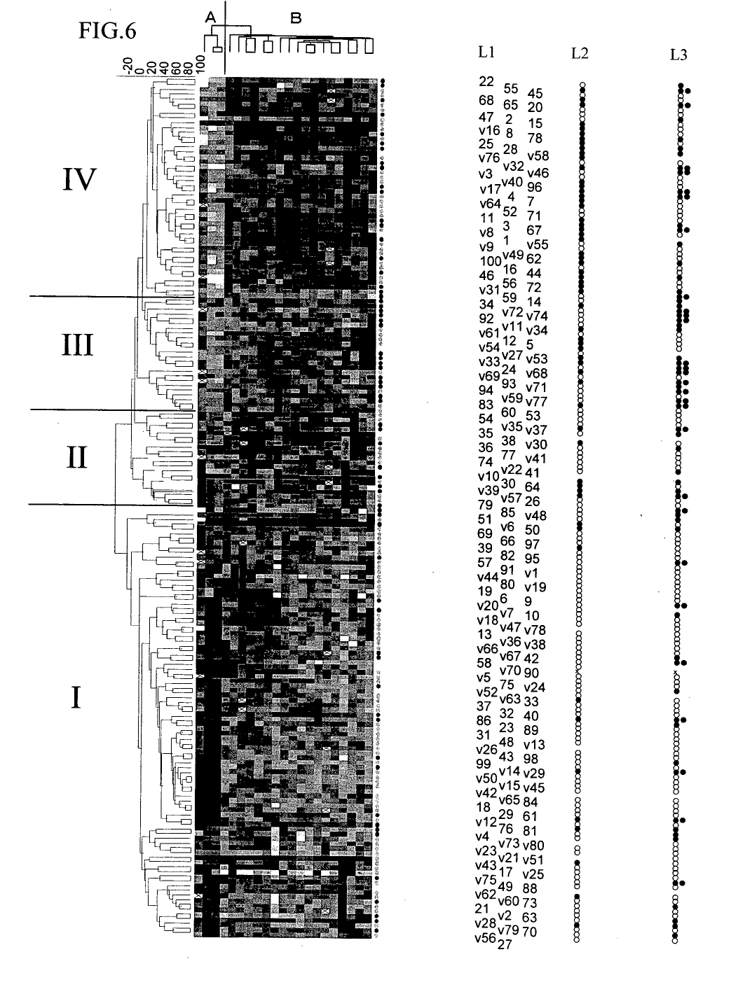

Then, cluster analysis was conducted based on the expression patterns of those isolated genes. FIG. 6 schematically shows the results, in which 179 cases are arranged vertically and 21 gene names are arranged horizontally. The gene names are, for group A from left to right, GS7435, GS2307 and GS2828. For group B, from left to right, GS2632, GS7288, GS6601, GS7583, GS7116, GS7715, GS6770, GS2471, GS6711, GS1176, GS7001, GS690, GS1472, GS6784, GS7012, GS7632, GS1957 and GS7264. Each cell (square) indicates the state of expression of the gene. A white square indicates a high expression, a black square indicates a low expression, and a gray square indicates an intermediate expression level. The lighter the shade of gray, the higher the expression, and the darker the shade of gray, the lower the expression. In the present example, a low expression means the expression level of not less than −1.3 and not more than −0.3; an intermediate expression means more than −0.3 and less than 0.3; and a high expression means not less than 0.3 and not more than 1.3. “Expression level” here refers to the level calculated by standardizing measurements with a median value, limiting the standardized values within an upper value of 20 and a lower value of 0.5, and then transforming the values into a logarithm.

In FIG. 6, the numerical values in the column “L1” indicate specimen numbers given for the sake of convenience. The open circles and closed circles in the column of “L2” indicate whether or not there is expression of the estrogen receptor: an open circle indicates positive and a closed circle negative. The open and closed circles in the column of “L3” indicate whether or not there is lymph node metastasis and if there is, how many: an open circle indicates zero; a closed circle indicates one to three; and double closed circles indicate four or more. As shown in FIG. 6, the cases can be divided into four groups (I, II, III and IV), while the gene groups can be divided into two groups (A and B).

Table 5 shows the relationship between the case groups and the gene groups (Groups A and B).

TABLE 5

| Case | | | |
|---|---|---|---|
| group | Group A | Group B | Estrogen receptor |
| I | Low expression | High expression | Mostly positive |
| II | Low expression | Low expression | Mixture of positive |
| | | | and negative |
| III | High expression | High expression | Mixture of positive |
| | | | and negative |
| IV | High expression | Low expression | Mostly negative |

Table 6 shows the relationship between the case groups and lymph node metastasis.

TABLE 6

| Case | Metastasis present | Metastasis absent | |
|---|---|---|---|
| group | (one or more) | (zero) | Metastasis (%) |
| I | 22 | 61 | 26.5 |
| II | 8 | 10 | 44.4 |
| III | 18 | 9 | 66.6 |
| IV | 16 | 24 | 40 |
| Total | 64 | 104 | 38.1 |

Group I has less metastasis and Group III has more metastasis.

Similarly, when genes satisfying the following equation:
(variance in cancer specimens)/(variance in normal specimens)≧1.15
are selected based on differences in the level of expression between an estrogen receptor-positive and negative group, the genes having nucleotide sequences 1-27 from Table 1 are selected.

Further, if the selection is set such that
(variance in cancer specimens)/(variance in normal specimens)≧1.10,
genes other than those having nucleotide sequences 1-27 from Table 1 are additionally selected. Thus, by subjecting the levels of expression of these selected genes to multivariate analysis and dividing the cases into several groups in a similar manner, information useful for predicting prognosis can be obtained.

### (EXAMPLE 3)

### Estimation of Metastasis and Early Recurrence of Breast Cancer

In the present example, the prediction of metastasis and early recurrence was analysed in 301 cases of breast cancer. Cluster analysis was conducted by using the 21 genes selected in Example 2. The results were as shown below.

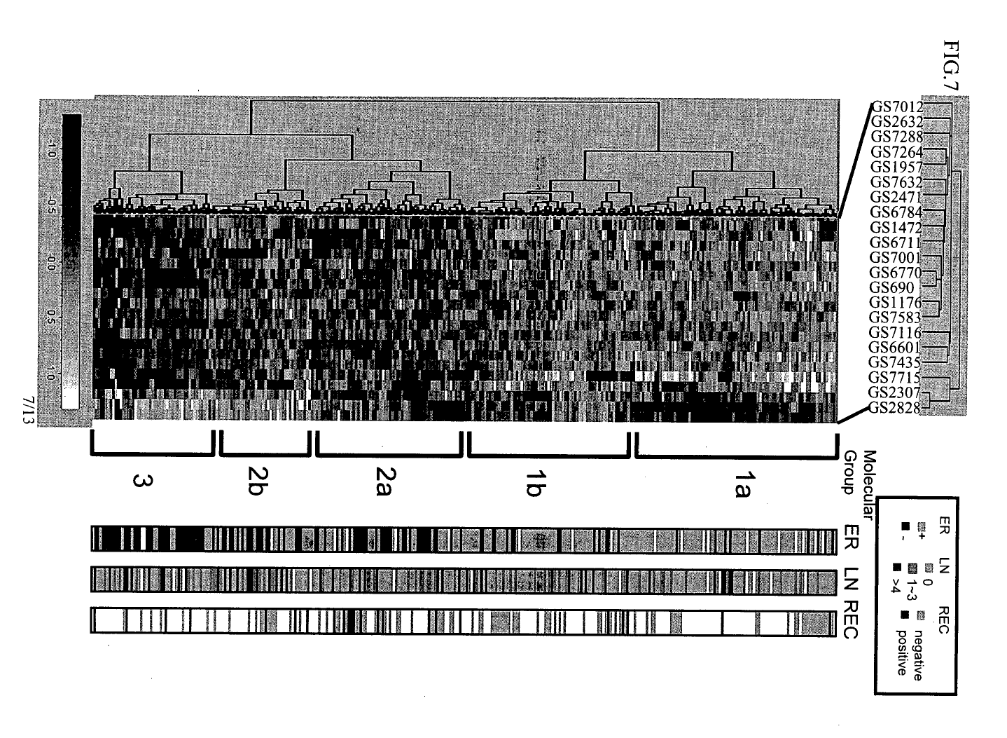

1. Estrogen Receptor-Positive Group (Molecular Groups 1a and 1b in FIG. 7)

In this group, lymph node metastasis was observed in 45 out of 143 cases (31%). Early recurrence was observed in 5 out of 60 cases (8%).

2. Estrogen Receptor-Positive and Negative Mixed Groups (Molecular Group 2a and 2b in FIG. 7)

Lymph node metastasis was observed in 47 of 101 cases (47%), and early recurrence was observed in 14 out of 49 cases (29%).

3. Estrogen Receptor-Negative Group (Molecular Group 3 in FIG. 7)

Lymph node metastasis was observed in 21 of 44 cases (48%), and early recurrence was observed in 4 of 10 cases (40%).

Those results are shown in Table 7.

TABLE 7

| | Lymph node | |
|---|---|---|
| | metastasis | Early recurrence |
| Estrogen receptor-positive group | 45/143 (31%) | 5/60 (8%) |
| Estrogen receptor-positive and | 47/101 (47%) | 14/49 (29%) |
| negative mixed groups | | |
| Estrogen receptor-negative group | 21/44 (48%) | 4/10 (40%) |

In FIG. 7, ER designates an estrogen receptor (positive +, negative −), LN designates lymph node metastasis (number), and REC designates recurrence (positive or negative).

FIG. 7 and Table 7 indicate that the chances of early recurrence in the estrogen receptor-negative group can be high. Early recurrence invariably results in death, so the results obtained by the method of the invention can provide important information for medical prognosis.

### (EXAMPLE 4)

### Prediction of Breast Cancer

By combining the molecular groups for the prediction of cancer obtained in Example 3 and known clinical parameters, prognosis of breast cancer can be carried out as accurately as possible. Table 8 shows the clinical parameters and their prognostic significance determined by Cox regression analysis.

TABLE 8

| | Risk/reference | Univariate | | Multivariate | |
|---|---|---|---|---|---|
| | factors (R.R.) | R.R. | p value | R.R. | p value |
| Menstrual status | Before/after | 1.4 | 0.497 | | |
| Tumor size | >2/<2 (cm) | 1.8 | 0.187 | | |
| Lymph node | Positive/negative | 2.9 | 0.012 | 2.3 | 0.048 |
| metastasis | | | | | |
| Histological grade | III/I and II | 3.1 | 0.016 | | 0.218 |
| Estrogen receptor | Negative/positive | 2.6 | 0.030 | | 0.159 |
| Molecular group | 2 and 3/1 | 4.0 | 0.006 | 3.2 | 0.022 |

Based on the information in Table 8, prognosis of a cancer specimen can be determined from a plurality of parameters. Particularly, the R.R. value (degree of risk relative to early recurrence) is highest in the molecular group. Thus, cancer can be predicted more accurately by the molecular group than by the conventional clinical parameters.

### (EXAMPLE 5)

### Adaptor-Tagged Competitive PCR using a Colon Cancer Specimen

The expression levels of 1536 genes were measured in 115 cases (105 cases of colon cancer and 10 cases of normal tissue) by the adaptor-tagged competitive PCR method.

PCR reaction and the quantitative determination of the gene expression levels were carried out in the same way as in Example 1.

### (EXAMPLE 6)

### Selection of Genes by Cluster Analysis

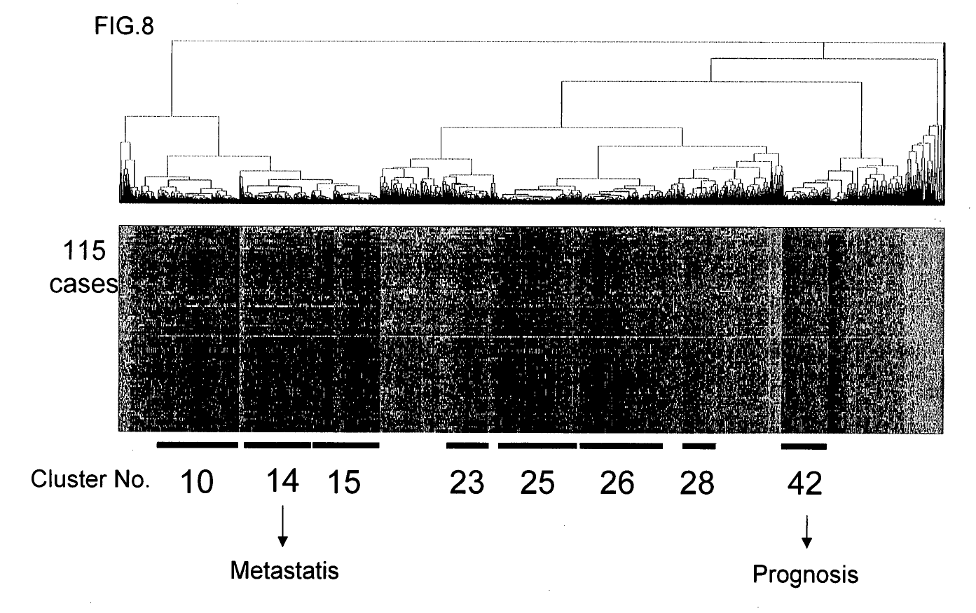

Cluster analysis was performed by using the expression patterns of the above 1536 genes. FIG. 8 schematically shows the result. FIG. 7 shows the 115 cases listed vertically and the results of expression of the 1536 genes listed horizontally. As in FIG. 6, each cell (square) indicates the state of expression levels of a gene. A white cell (blank square) indicates a high expression level, a black cell (blacked-out square) indicates a low expression level, and gray indicates an intermediate level of expression. A lighter shade of gray indicates a higher expression level, and a darker shade of gray indicates a lower expression level. A low expression level refers to expression levels of not less than −1.301 and not more than −0.3. An intermediate expression level refers to expression levels more than −0.3 and less than 0.3. A high expression level refers to expression levels not less than 0.3 and not more than 1.301. As a result of the cluster analysis, the 1536 genes were divided into 88 clusters.

From the thus cluster-analyzed genes, cluster No. 14 in FIG. 8 was selected as a metastasis (M) cluster, while clusters Nos. 42-44 were selected as prognosis (P) clusters. Clusters No. 14 and Nos. 42-44 were selected because, when cluster analysis as described in Example 7 below was performed in advance, those clusters had been predicted to be related to metastasis and prognosis, respectively.

Table 2 above shows the genes contained in cluster No. 14. In Table 2, genes of sequences Nos. 28 to 153 are those selected as M cluster. On the other hand, table 3 above shows the genes contained in clusters Nos. 42 to 44. In Table 3, genes of sequences Nos. 154 to 289 are those selected as P cluster.

### (EXAMPLE 7)

### Multivariate Analysis (Cluster Analysis)

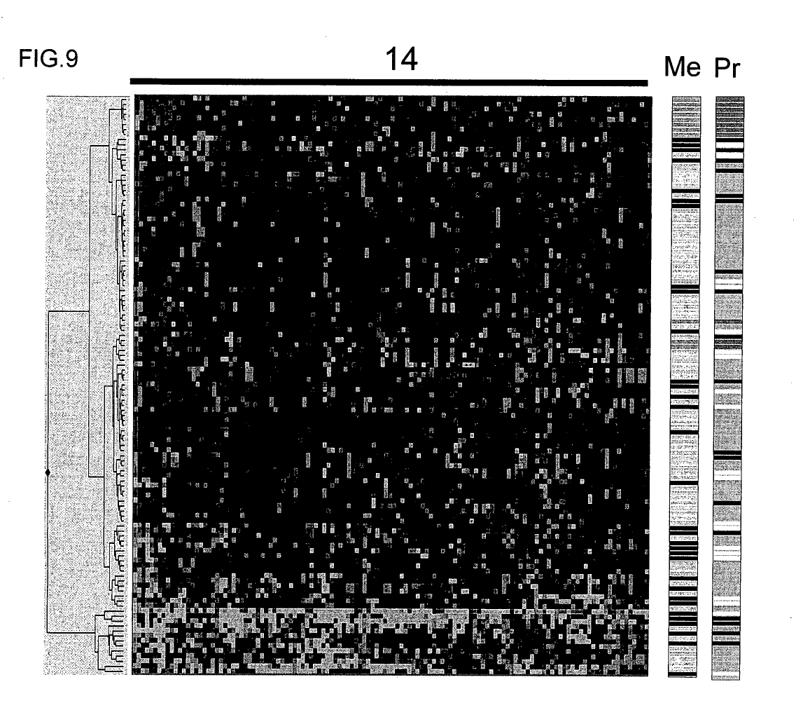

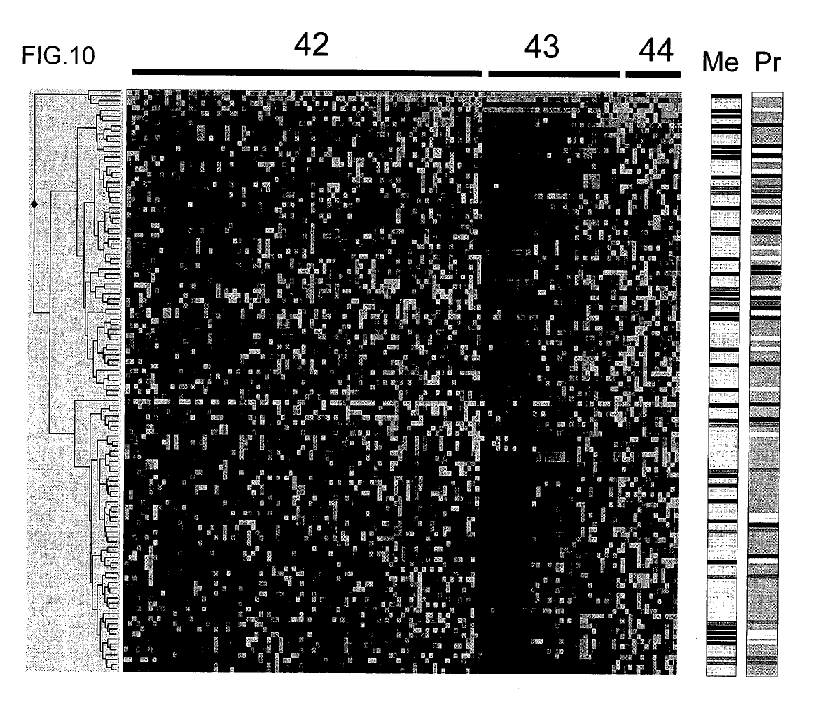

Cluster analysis was performed on the genes selected in Example 6. FIG. 9 shows the cluster analysis of the genes belonging to M cluster. FIG. 10 shows the cluster analysis of the genes belonging to P cluster. In FIG. 9, 115 cases are arranged vertically and 126 genes of M cluster are arranged horizontally. Each cell (square) indicates the expression level of a gene. Me indicates metastasis, and Pr indicates prognosis. The colors in a column Me are such that black represents a metastasized cancer specimen, white a cancer specimen without metastasis, and gray a normal specimen. The colors in a column Pr are such that black indicate a cancer specimen with poor prognosis, white a cancer specimen with intermediate prognosis, light gray a cancer specimen with good prognosis, and dark gray a normal specimen. “Poor” prognosis means that the patient died of cancer within two years of prognosis following the treatment of the primary cancerous lesion of colon cancer. “Intermediate” prognosis likewise means that the patient either died of cancer within two to five years or, if he is alive, less than four years of the observation period have passed. “Good” prognosis means that the patient is alive and four or more years of observation period have passed.

FIG. 10 shows 115 cases arranged vertically and 136 genes of P cluster arranged horizontally. Numerals 42, 43 and 44 indicate the cluster numbers in the cluster analysis shown in FIG. 8. Each cell (square) indicates the expression level of a gene. The colors in a column Me on the right are coded such that black, white and gray indicate a metastasized cancer specimen, a cancer specimen without metastasis, and a normal specimen, respectively. The colors in a column Pr on the right are coded such that black, white, light gray and dark gray indicate a cancer specimen with poor prognosis, a cancer specimen with intermediate prognosis, a cancer specimen with good prognosis, and a normal specimen, respectively.

FIGS. 9 and 10 show that in M cluster, cases of metastasized specimens are concentrated at the bottom, while in P cluster, specimens with bad prognosis and metastasized specimens are concentrated at the top. It was believed, therefore, that the specimens were successfully classified into individual groups by cluster analysis using these genes belonging to M or P clusters according to relevance to metastasis and clinical prognostic factors. Thus, the inventors selected M cluster as a group related to metastasis and P cluster as a group related to prognosis and metastasis.

### (EXAMPLE 8)

### Multivariate Analysis (Principal Component Analysis)

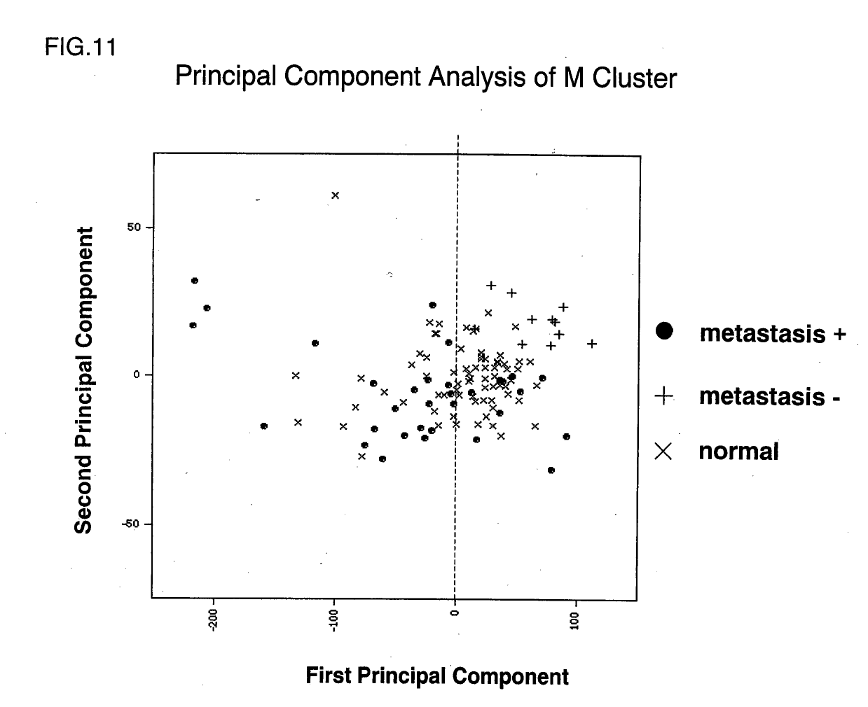

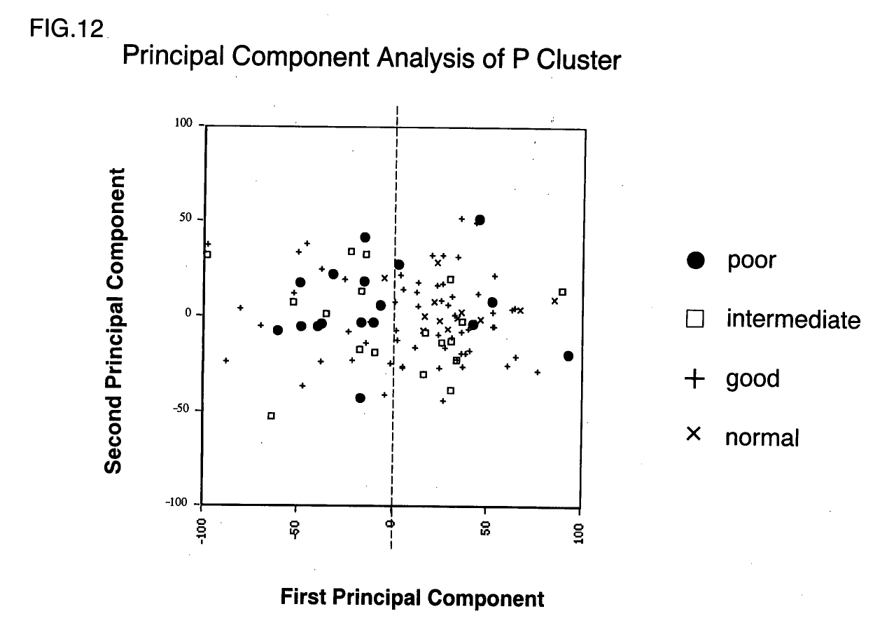

Principal component analysis was carried out in order to determine statistically significant values of the results of cluster analysis of M and P clusters performed in Example 7. The results are shown in FIGS. 11 and 12. In FIG. 11, a metastasized cancer specimen is indicated by “•”, a cancer specimen without metastasis by “+”, and a normal specimen by “×”. In FIG. 12, a specimen with poor prognosis is indicated by “•”, a specimen with intermediate prognosis by a square, a specimen with good prognosis by “+” and a normal specimen by “×”.

As a result of principal component analysis, a border line can be drawn, as indicated by the dashed line in FIGS. 11 and 12. Based on these figures, the values shown in Table 9 were determined. The border line indicates an average value of a first principal component.

TABLE 9
Factor scores of first principal component

| | | Negative | Positive | χ² test | |
|---|---|---|---|---|---|
| P cluster | | | | | |
| Prognosis | Poor(%) | 39.3 | 11.1 | 8.45 | ο |
| | (poor/good) | (13/20) | (5/45) | | |
| Metastasis | Positive(%) | 47.7 | 18.3 | 8.96 | ο |
| | (positive/negative) | (21/23) | (11/49) | | |
| M cluster | | | | | |
| Prognosis | Poor(%) | 33.3 | 19.6 | 0.017 | X |
| | (poor/good) | (8/23) | (10/42) | | |
| Metastasis | Positive(%) | 46.7 | 18.6 | 10.2 | ο |
| | (positive/negative) | (22/24) | (10/49) | | |

The values in Table 9 indicate the evaluation of each cluster wherein when the value of a first principal component is positive value, the prediction for prognosis or metastasis is positive, and when it is negative value, the prediction is negative. The evaluation is carried out by an χ² test (χ²=6.63 when p=0.01). A value exceeding this χ² value indicates that the individual ratios are significantly different and useful for cancer prediction. Accordingly, the genes in P cluster are useful for predicting both prognosis and metastasis, while the genes in M cluster are useful for predicting metastasis.

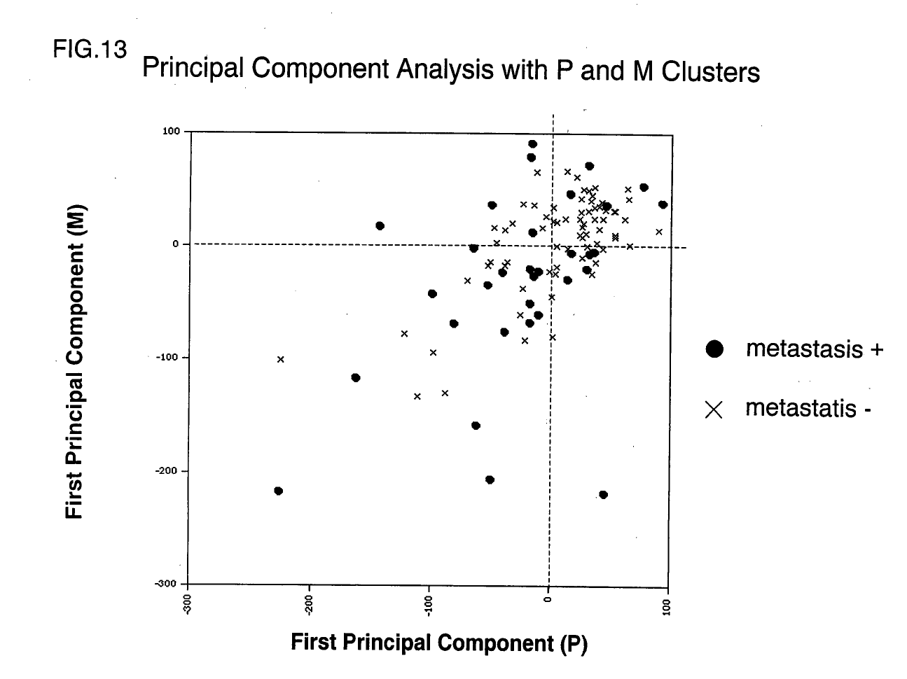

The present inventors further conducted principal component analysis by combining M and P clusters. The results are shown in FIG. 13, in which the first principal component on the horizontal axis is that of the P cluster, and the first principal component on the vertical axis is that of the M cluster. A metastasized cancer specimen is indicated by “•” and a cancer specimen without metastasis by “33 ”. As a result of the principal component analysis, a border line can be drawn in the figure, as indicated by the dashed line. This border line indicates an average value of the first principal components. Based on FIG. 13, the values shown in Table 10 were determined.

TABLE 10
Statistical significance of a combined analysis

| P and M | | 2^{nd}, 3^{rd} and | | | |
|---|---|---|---|---|---|
| clusters | | 4^{th} quadrants | 1^{st} quadrant | χ² test | |
| Prognosis | Poor(%) | 31.8 | 10.2 | 4.46 | X |
| | (poor/good) | (14/30) | (4/35) | | |
| Metastasis | Positive(%) | 45.0 | 11.3 | 11.9 | ο |
| | (positive/negative) | (27/33) | (5/39) | | |
| | | factor score of 1st component | | | |
| M cluster | | Negative | Positive | χ² test | |
| Metastasis | Positive(%) | 46.7 | 18.6 | 10.2 | ο |
| | (positive/negative) | (22/24) | (10/49) | | |

In Table 10, the quadrants refer to the parts divided by the border lines as shown in FIG. 13. The first, second, third and fourth quadrants respectively correspond to the upper-right, lower-right, upper-left and lower-left parts of FIG. 13.

Table 10 indicates that, of the genes belonging to P and M clusters, specimens classified in the first quadrant as a result of multivariate analysis of their gene expression patterns can have a low probability (11.3%) of metastasis, while the genes classified into the other quadrants have a higher probability of metastasis. With regard to metastasis, the value of χ² test is higher in the case of combining M and P clusters than in the case of using M cluster alone. Thus, it is suggensted that based on this combination, the prediction of metastasis of colon cancer can be judged more efficiently. Because prognosis of colon cancer cannot be predicted with statistical significance by the combination of M and P clusters, as shown in Table 10, it is believed to be preferable to use the genes of P cluster, as shown in Table 9.

All publications, patents and patent applications cited herein are incorporated herein by reference in their entirety.

### INDUSTRIAL APPLICABILITY

The present invention provides a cancer predicting method and a drug design method. The method according to the invention is useful for genetic diagnosis for evaluating cancer malignancy. The results according to the method of the present invention are useful for designing drugs.

1. A method for classifying cancer, comprising the steps of:
(a) collecting genes from specimens and measuring an expression level thereof;
(b) selecting at least one of the measured genes;
(c) subjecting the measurements of expression level for the selected gene to multivariate analysis; and
(d) classifying the specimens into groups of similar gene expression patterns by using the result of multivariate analysis as an indicator.

2. A cancer predicting method comprising the steps of:
(a) collecting genes from specimens and measuring an expression level thereof;
(b) selecting at least one of the measured genes;
(c) subjecting the measurements of expression level for the selected gene to multivariate analysis;
(d) classifying the specimens into groups of similar gene expression patterns by using the result of multivariate analysis as an indicator; and
(e) predicting the state of cancer based on the result of classification.

3. The method according to claim 2, further comprising the steps of determining an expression pattern characteristic of a state of cancer, and comparing the expression pattern of a gene collected from a cancer specimen for which cancer is to be predicted with the characteristic expression pattern.

4. The method according to claim 1 or 2, wherein the state of cancer is at least one selected from the group consisting of presence or absence of cancer, malignancy of cancer, presence or absence of metastasis of cancer, and presence or absence of recurrence of cancer.

5. The method according to claim 4, wherein the metastasis is lymph node metastasis.

6. The method according to claim 4, wherein the recurrence is early recurrence.

7. The method according to claim 1 or 2, wherein the at least one gene selected is gene group I of genes comprising nucleotide sequences 1 to 27 from Table 1, gene group II of genes comprising nucleotide sequences 28 to 153 from Table 2, and/or gene group III of genes comprising nucleotide sequences 154 to 289 from Table 3.

8. The method according to claim 1 or 2, wherein the at least one gene selected is a combination of at least one gene selected from gene group I of genes comprising nucleotide sequences 1 to 27 from Table 1, from gene group II of genes comprising nucleotide sequences 28 to 153 from Table 2, and/or from gene group III of genes comprising nucleotide sequences 154 to 289 from Table 3, and at least one gene other than those in gene groups I, II and III.

9. The method according to claim 1 or 2, wherein the classification is based on a hormone receptor-positive group and/or negative group as an indicator.

10. The method according to claim 9, wherein the hormone receptor is an estrogen receptor.

11. The method according to claim 1 or 2, wherein the cancer is selected from the group consisting of breast cancer, stomach cancer, esophageal cancer, oral cancer, colon cancer, rectal cancer, anal cancer, pancreatic cancer, lung cancer, renal cancer, bladder cancer, ovarian cancer, uterine cancer, skin cancer, melanoma, central nervous tumor, peripheral nervous tumor, gum cancer, pharyngeal cancer, maxillary and jowl cancer, liver cancer, prostate cancer, leukemia, multiple myeloma, and malignant limphoma.

12. The method according to claim 11, wherein the cancer is breast cancer or colon cancer.

13. The method according to claim 1 or 2, wherein the multivariate analysis is a cluster analysis.

14. A drug design method, comprising designing a drug for suppressing the expression of a gene that is expressed in a specimen whose state of cancer has been predicted to be at high-risk by the method according to one of claims 1 to 13.

15. The method according to claim 14, wherein the gene is a gene having a nucleotide sequence which is selected from the group consisting of nucleotide sequences 4, 7 and 20 from Table 1, nucleotide sequences 28, 29, 31, 32, 35, 43, 49-53, 67, 70, 72, 73, 75-79, 81, 84, 86-92, 94-99, 104-111, 113, 114, 117 and 122-153 from Table 2, and nucleotide sequences 155, 162, 163, 167-169, 171, 172, 174, 175, 177-180, 188., 190, 193, 198, 211, 222, 242-253, 255-257, 259-261, 263 and 265 from Table 3, or a combination thereof.

16. The method according to claim 14 or 15, wherein the drug is an antisense nucleic acid.

17. A drug design method, comprising designing a drug for enhancing the expression of a gene that is expressed in a specimen whose state of cancer has been predicted to be at high-risk by the method according to one of claims 1 to 13.

18. The method according to claim 17, wherein the gene is a gene having a nucleotide sequence which is selected from the group consisting of nucleotide sequences 1-3, 5, 6, 8-19 and 21 from Table 1, nucleotide sequences 30, 33, 34, 36-42, 44-48, 54-66, 68, 69, 71, 74, 80, 82, 83, 85, 93, 100-103, 112, 115, 116 and 118-121 from Table 2, and nucleotide sequences 154, 156-161, 164-166, 170, 173, 176, 181-187, 189, 191, 192, 194-197, 199-210, 212-221, 223-241, 254, 258, 262, 264 and 266-289 from Table 3, or a combination thereof.

19. The method according to claim 17 or 18, wherein the drug is a targeting vector.

20. A program for having a computer function as a cancer-state prediction system comprising means of analyzing the expression level of a gene collected from a primary cancerous lesion, and means of identifying the state of cancer by using the result of analysis as an indicator.

21. A computer-readable recording medium in which is stored a program for having a computer function as a cancer-state prediction system comprising means of analyzing the expression level of a gene collected from a primary cancerous lesion, and means of identifying the state of cancer by using the result of analysis as an indicator.
